(19) United States
(12) Patent Application Publication
(10) Pub. No.: US 2025/0319095 A1
(43) Pub. Date: Oct. 16, 2025

(54) USE OF BENZODIAZEPINES TO INCREASE SENSITIVITY TO PSILOCYBIN FOLLOWING A CHRONIC SSRI REGIMEN

(71) Applicant: Compass Pathfinder Limited, Altrincham, Cheshire (GB)

(72) Inventor: Molly Tabitha HICKEY, Altrincham (GB)

(21) Appl. No.: 18/032,320

(22) PCT Filed: Oct. 21, 2021
(86) PCT No.: PCT/EP2021/079287

§ 371 Date: Apr. 17, 2023

Related U.S. Application Data
(60) Provisional application No. 63/094,624, filed on Oct. 21, 2020.

(51) Int. Cl. A61K 31/5513; A61K 31/4525; A61K 31/675

(52) U.S. Cl. CPC A61K 31/5513; A61K 31/4525; A61K 31/675

(57) ABSTRACT

The disclosure provides methods for treating a patient in need thereof comprising co-administering to the patient a therapeutically-effective dose of psilocybin and one or more benzodiazepines. The present disclosure also provides methods to reduce the SSRI washout period in patients prior to administration of psilocybin therapy.

### CROSS-REFERENCE TO RELATED APPLICATIONS

This application is a National Stage Entry of International Patent Application No. PCT/EP2021/079287, filed October 21, titled “Use of Benzodiazepines to Increase Sensitivity to Psilocybin Following a Chronic SSRI Regiment,” which claims priority to U.S. Application No. 63/094,624, filed Oct. 21, 2020, which are hereby incorporated by reference in their entirety herein.

### BACKGROUND

Psychedelics show significant promise in the treatment of several psychiatric indications. Current psychedelic administration guidelines recommend that patients on a chronic selective serotonin reuptake inhibitor (SSRI) regimen cease any SSRI therapy for at least 2 weeks prior to administration of psilocybin therapy, due to the observed impact of SSRIs on sensitivity to psilocybin. This leaves patients at risk, with no pharmacological support during this period of ‘washout’ and exposed to SSRI withdrawal symptoms. Therefore, there is a need to reduce this washout period such that patients who require psilocybin therapy would be unsupported for a shorter duration, reducing risk to the patient, improving the patient's experience, and increasing patient compliance.

### SUMMARY

The present disclosure provides methods to reduce the SSRI washout period in patients prior to administration of psilocybin therapy.

Provided herein is a method of administering psilocybin to a subject in need thereof, wherein prior to administration of psilocybin the subject was on a selective serotonin reuptake inhibitor (SSRI) therapy regimen comprising: a) ceasing SSRI therapy 1 to 35 days prior to administration of psilocybin; b) administering one or more benzodiazepines at least once daily to the subject starting at least 1 to 35 days prior to administration of psilocybin; and) administering psilocybin to the subject.

In some embodiments, the SSRI therapy is ceased during a titration period, wherein during the titration period the dose of SSRI is reduced from a maintenance dose to cessation.

In some embodiments, one or more benzodiazepines is administered during the SSRI titration period.

In some embodiments, one or more benzodiazepines is administered after cessation of the SSRI.

In some embodiments, one or more benzodiazepines is administered before the SSRI titration period.

In some embodiments, SSRI therapy is ceased immediately, wherein immediate cessation of an SSRI does not comprise a titration period.

In some embodiments, one or more benzodiazepines are administered after cessation of SSRI therapy.

In some embodiments, one or more benzodiazepines are administered before cessation of SSRI therapy.

In some embodiments, SSRI therapy is ceased 29 to 35 days prior to administration of psilocybin and one or more benzodiazepines are administered starting 1 to 28 days prior to administration of psilocybin.

In some embodiments, SSRI therapy is ceased 22 to 35 days prior to administration of psilocybin and one or more benzodiazepines are administered starting 1 to 21 days prior to administration of psilocybin.

In some embodiments, SSRI therapy is ceased 15 to 35 days prior to administration of psilocybin and one or more benzodiazepines are administered starting 1 to 14 days prior to administration of psilocybin.

In some embodiments, SSRI therapy is ceased 8 to 35 days prior to administration of psilocybin and one or more benzodiazepines are administered starting 1 to 7 days prior to administration of psilocybin.

In some embodiments, SSRI therapy is ceased 29 to 35 days prior to administration of psilocybin and one or more benzodiazepines are administered starting at least 35 days before administration of psilocybin.

In some embodiments, SSRI therapy is ceased 22 to 35 days prior to administration of psilocybin and one or more benzodiazepines are administered starting at least 35 days before administration of psilocybin.

In some embodiments, SSRI therapy is ceased 15 to 35 days prior to administration of psilocybin and one or more benzodiazepines are administered starting at least 35 days before administration of psilocybin.

In some embodiments, SSRI therapy is ceased 8 to 35 days prior to administration of psilocybin and one or more benzodiazepines are administered starting at least 35 days before administration of psilocybin.

In some embodiments, SSRI therapy is ceased in 1 to 35 days prior to administration of psilocybin and one or more benzodiazepines is administered 1 to 28 days prior to administration of psilocybin.

In some embodiments, SSRI therapy is ceased in 1 to 35 days prior to administration of psilocybin and one or more benzodiazepines is administered 1 to 14 days prior to administration of psilocybin.

In some embodiments, SSRI therapy is ceased in 1 to 35 days prior to administration of psilocybin and one or more benzodiazepines is administered 1 to 7 days prior to administration of psilocybin.

In some embodiments, SSRI therapy is ceased in 1 to 28 days prior to administration of psilocybin and one or more benzodiazepines is administered 1 to 35 days prior to administration of psilocybin.

In some embodiments, SSRI therapy is ceased in 1 to 28 days prior to administration of psilocybin and one or more benzodiazepines is administered 1 to 28 days prior to administration of psilocybin.

In some embodiments, SSRI therapy is ceased in 1 to 28 days prior to administration of psilocybin and one or more benzodiazepines is administered 1 to 14 days prior to administration of psilocybin.

In some embodiments, SSRI therapy is ceased in 1 to 28 days prior to administration of psilocybin and one or more benzodiazepines is administered 1 to 7 days prior to administration of psilocybin.

In some embodiments, SSRI therapy is ceased in 1 to 14 days prior to administration of psilocybin and one or more benzodiazepines is administered 1 to 35 days prior to administration of psilocybin.

In some embodiments, SSRI therapy is ceased in 1 to 14 days prior to administration of psilocybin and one or more benzodiazepines is administered 1 to 28 days prior to administration of psilocybin.

In some embodiments, SSRI therapy is ceased in 1 to 14 days prior to administration of psilocybin and one or more benzodiazepines is administered 1 to 21 days prior to administration of psilocybin.

In some embodiments, SSRI therapy is ceased in 1 to 14 days prior to administration of psilocybin and one or more benzodiazepines is administered 1 to 14 days prior to administration of psilocybin.

In some embodiments, SSRI therapy is ceased in 1 to 14 days prior to administration of psilocybin and one or more benzodiazepines is administered 1 to 7 days prior to administration of psilocybin.

In some embodiments, SSRI therapy is ceased in 1 to 7 days prior to administration of psilocybin and one or more benzodiazepines is administered 1 to 35 days prior to administration of psilocybin.

In some embodiments, SSRI therapy is ceased in 1 to 7 days prior to administration of psilocybin and one or more benzodiazepines is administered 1 to 28 days prior to administration of psilocybin.

In some embodiments, SSRI therapy is ceased in 1 to 7 days prior to administration of psilocybin and one or more benzodiazepines is administered 1 to 21 days prior to administration of psilocybin.

In some embodiments, SSRI therapy is ceased in 1 to 7 days prior to administration of psilocybin and one or more benzodiazepines is administered 1 to 14 days prior to administration of psilocybin.

In some embodiments, SSRI therapy is ceased in 1 to 7 days prior to administration of psilocybin and one or more benzodiazepines is administered 1 to 7 days prior to administration of psilocybin.

In some embodiments, the maintenance SSRI daily dose is reduced by at least about 10% every three to four days.

In some embodiments, the maintenance SSRI daily dose is reduced by at least about 25% every three to four days.

In some embodiments, the maintenance SSRI daily dose is reduced by at least about 50% every three to four days.

In some embodiments, the maintenance SSRI daily dose is reduced by at least about 10% every week.

In some embodiments, the maintenance SSRI daily dose is reduced by at least about 25% every week.

In some embodiments, the maintenance SSRI daily dose is reduced by at least about 50% every week.

In some embodiments, the maintenance SSRI daily dose is reduced by at least about 10% every other week.

In some embodiments, the maintenance SSRI daily dose is reduced by at least about 25% every other week.

In some embodiments, the maintenance SSRI daily dose is reduced by at least about 50% every other week.

In some embodiments, reducing the maintenance SSRI daily dose starts between 1 and 16 weeks before administration of psilocybin.

In some embodiments, reducing the maintenance SSRI daily dose starts between 1 and 12 weeks before administration of psilocybin.

In some embodiments, reducing the maintenance SSRI daily dose starts between 1 and 8 weeks before administration of psilocybin.

In some embodiments, reducing the maintenance SSRI daily dose starts between 1 and 4 weeks before administration of psilocybin.

In some embodiments, reducing the maintenance SSRI daily dose starts between 1 and 2 weeks before administration of psilocybin.

In some embodiments, the SSRI is ceased 14 days prior to administration of psilocybin.

In some embodiments, the SSRI titration period comprises: a) administering to the subject 60-90% of the subject's maintenance dose of one or more SSRIs for 21 to 35 days prior to administration of psilocybin; b) administering to the subject 40-60% of the subject's maintenance dose of one or more SSRIs for 14 to 20 days prior to administration of psilocybin; c) administering to the subject 25-40% of the subject's maintenance dose of one or more SSRIs for 7 to 13 days prior to administration of psilocybin; and d) administering to the subject 5-25% of the subject's maintenance dose of one or more SSRIs for 1 to 6 days prior to administration of psilocybin.

In some embodiments, the SSRI titration period comprises: a) administering to the subject 60-90% of the subject's maintenance dose of one or more SSRIs for 21 to 35 days prior to administration of psilocybin; b) administering to the subject 30-60% of the subject's maintenance dose of one or more SSRIs for 14 to 20 days prior to administration of psilocybin; c) administering to the subject 5-30% of the subject's maintenance dose of one or more SSRIs for 7 to 13 days prior to administration of psilocybin; and d) ceasing SSRI therapy 6 days prior to administration of psilocybin.

In some embodiments, the SSRI is selected from the group consisting of citalopram, escitalopram, paroxetine, sertraline, fluvoxamine, fluoxetine, and combinations thereof.

In some embodiments, the benzodiazepine is selected from the group consisting of alprazolam, chlordiazepoxide, clonazepam, diazepam, lorazepam, oxazepam, and combinations thereof.

In some embodiments, at least one side effect of SSRI washout is reduced after treating according to the methods of the disclosure.

In some embodiments, the at least one side effect of SSRI washout is selected from the group consisting of headache, asthenia, flu syndrome, fever, vasodilation, nausea, diarrhea, anorexia, dry mouth, dyspepsia, constipation, flatulence, vomiting, weight loss, insomnia, nervousness, anxiety, somnolence, dizziness, tremor, decreased libido, abnormal thinking, sweating, rash, pruritus, abnormal vision, dyspepsia, abdominal pain, fatigue, arthralgia, myalgia, somnolence, agitation, dysmenorrhea, decreased libido, yawning, upper respiratory tract infection, rhinitis, sinusitis, ejaculation disorder, ejaculatory delay, impotence, dysphoric mood, irritability, agitation, dizziness, sensory disturbances (e.g. paresthesias such as electric shock sensations), confusion, lethargy, emotional lability, and hypomania.

These and other embodiments are addressed in more detail in the detailed description set forth below.

### BRIEF DESCRIPTION OF THE DRAWINGS

FIG. 1 is a numbered structural formula of psilocybin.

FIG. 2A is a XRPD diffractogram of Polymorph A (GM764B).

FIG. 2B is a XRPD diffractogram of Polymorph A′ (JCCA2160F).

FIG. 2C is a XRPD diffractogram of Polymorph B (JCCA2160-F-TM2).

FIG. 2D is a XRPD diffractogram of a Hydrate A (JCCA2157E).

FIG. 2E is a XRPD diffractogram of an ethanol solvate (JCCA2158D).

FIG. 2F is a XRPD diffractogram of product obtained during development of the process (CB646-E) (top)—compared to the diffractograms Polymorph A′ (JCCA2160F) (middle) and Polymorph B (JCCA2160-TM2) (bottom).

FIG. 3A is a DSC and TGA thermograph of Polymorph A (GM764B).

FIG. 3B is a DSC and TGA thermograph of Polymorph A′ (JCCA2160F).

FIG. 3C is a DSC thermograph of Polymorph B (GM748A).

FIG. 3D is a DSC and TGA thermograph of Hydrate A (JCCA2157E).

FIG. 3E is a DSC and TGA thermograph of ethanol solvate (JCCA2158D).

FIG. 4 is a form phase diagram showing the inter-relationship of form in water-based systems.

FIG. 5 is a 1H NMR spectrum of psilocybin.

FIG. 6 is a ¹³C NMR spectrum of psilocybin.

FIG. 7 is a FT-IR Spectrum of psilocybin.

FIG. 8 is a Mass Spectrum of psilocybin.

FIG. 9A shows the effect of benzodiazepine pretreatment on ethovision activity to psilocybin at 5 minute intervals.

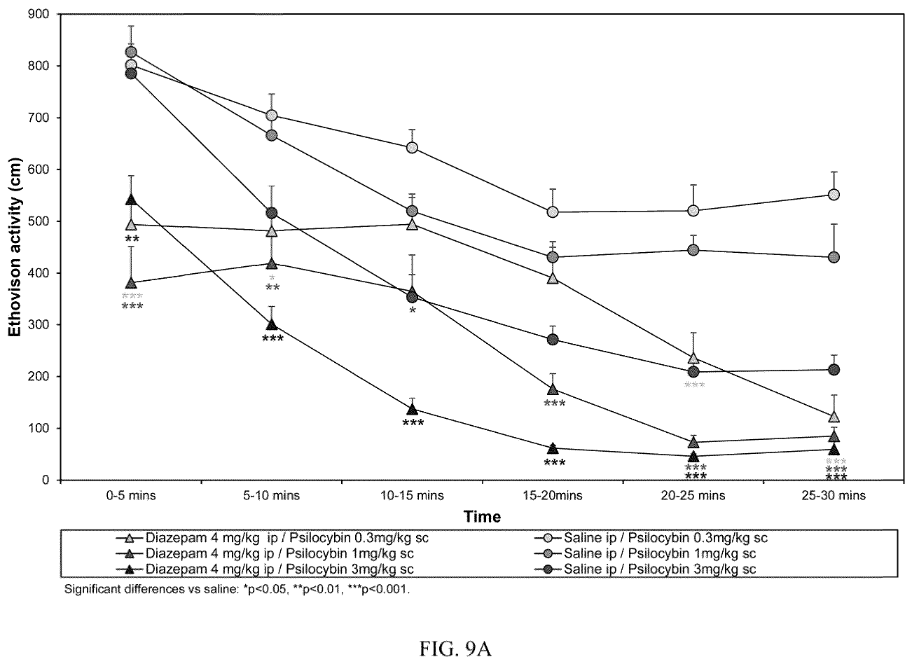

FIG. 9B shows the effect of benzodiazepine pretreatment on ethovision activity to psilocybin at 15 minute intervals.

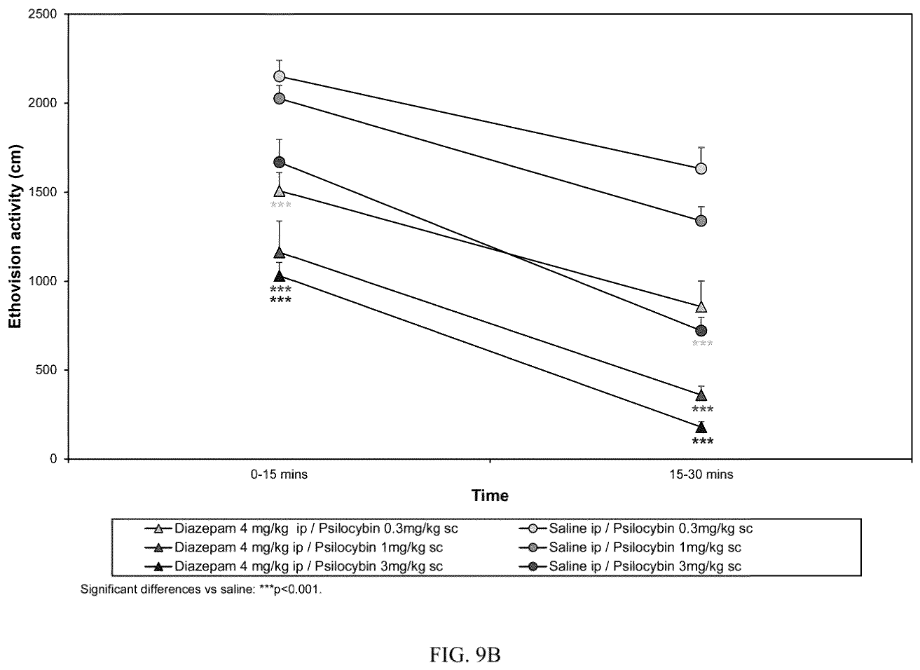

FIG. 9C shows the effect of benzodiazepine pretreatment on total ethovision activity to psilocybin.

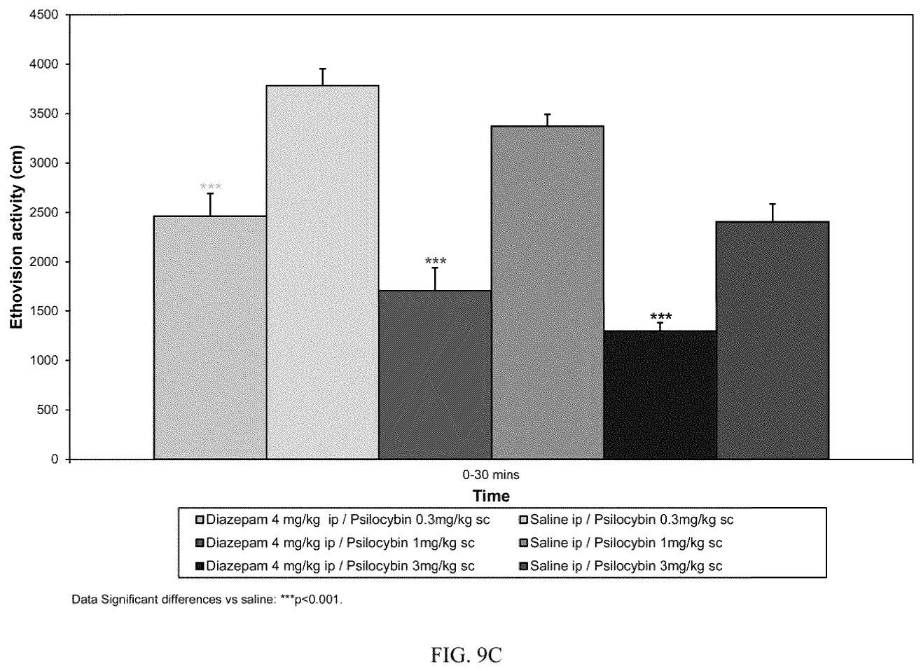

FIG. 10A shows the effect of benzodiazepine pretreatment on the head twitch response to psilocybin at 5 minute intervals.

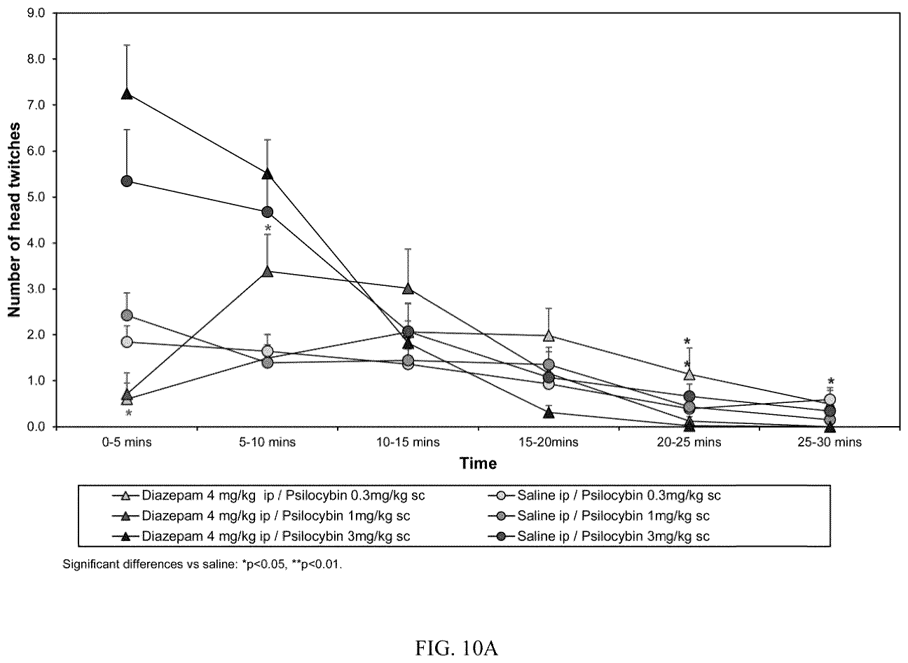

FIG. 10B shows the effect of benzodiazepine pretreatment on the head twitch response to psilocybin at 15 minute intervals.

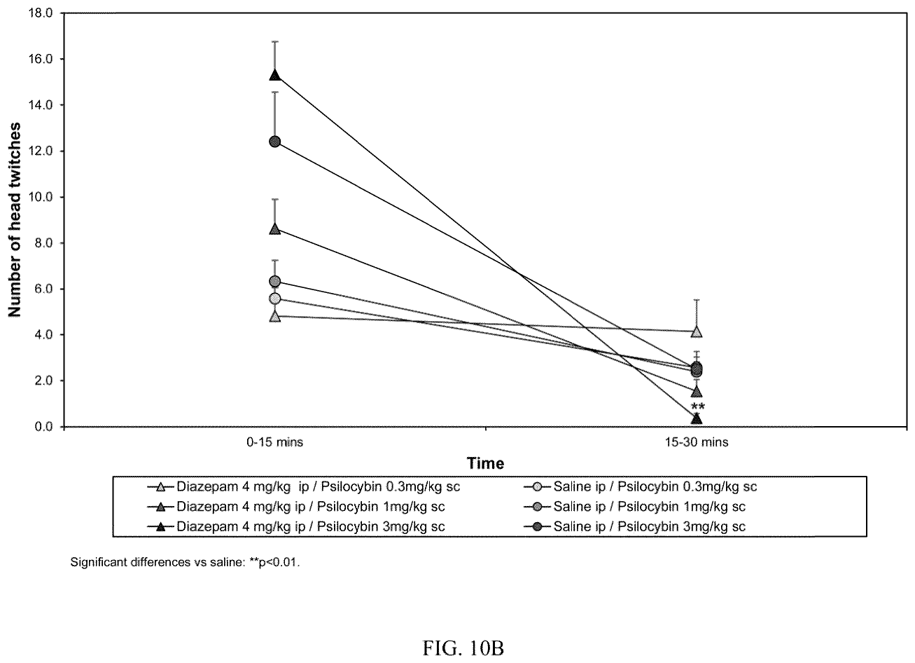

FIG. 10C shows the effect of benzodiazepine pretreatment on the total head twitch response to psilocybin.

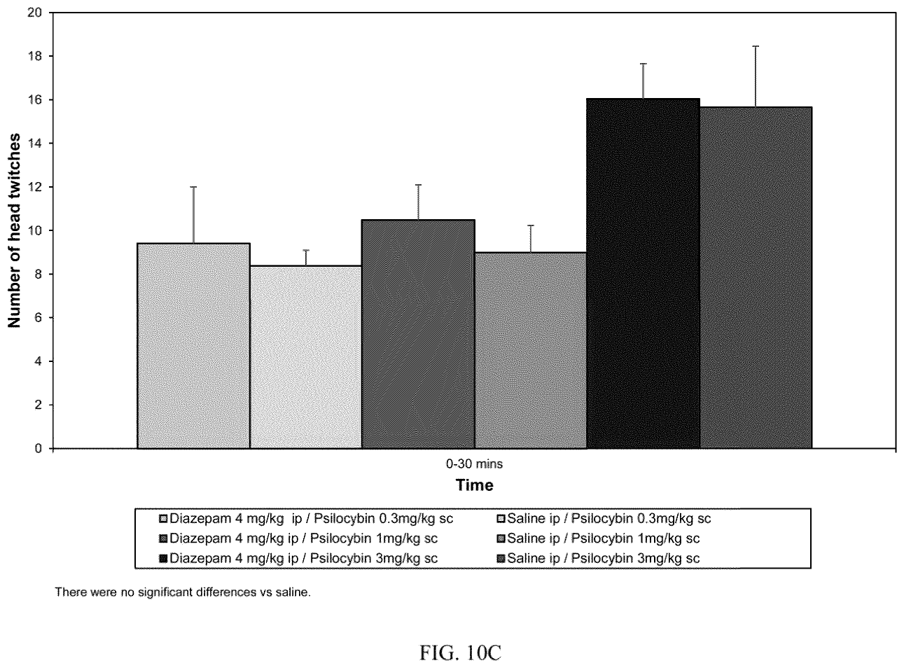

FIG. 11A shows the effect of chronic Benzodiazepine pretreatment on the head twitch response to psilocybin at 5 minutes intervals.

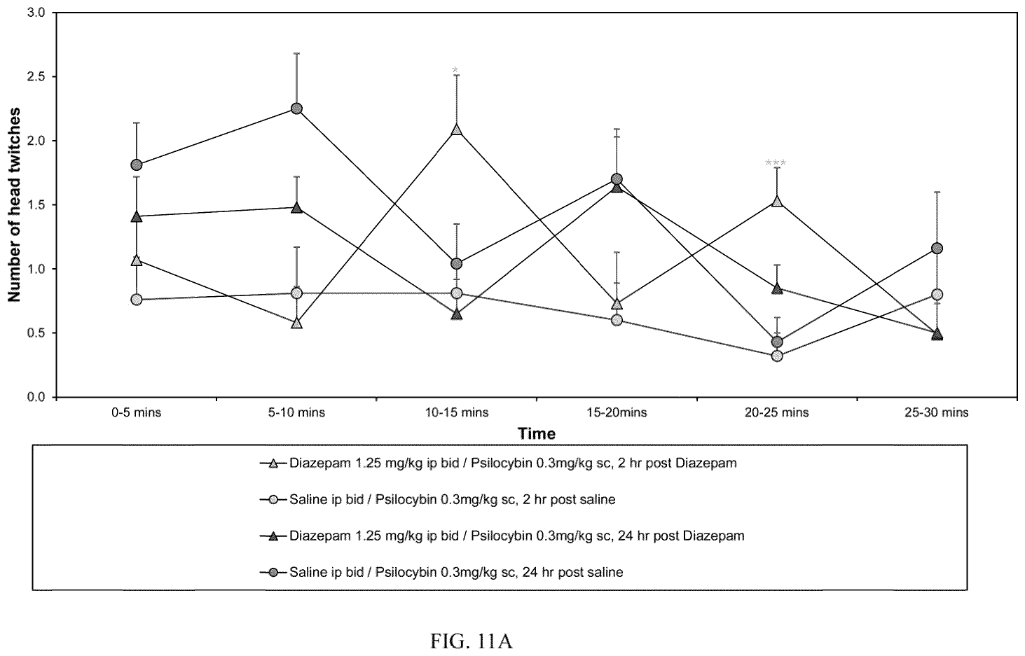

FIG. 11B shows the effect of chronic Benzodiazepine pretreatment on the total head twitch response.

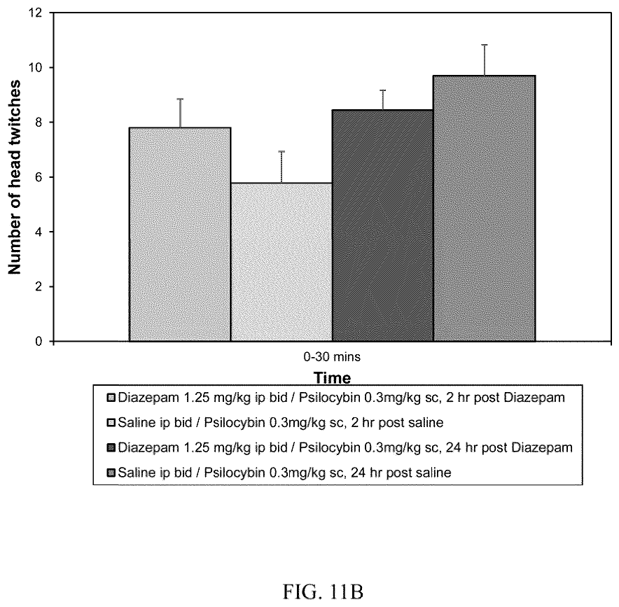

FIG. 12 shows the effect of chronic diazepam treatment (1.25 mg/kg ip, BID for 14 days) on 5HT_{2A} receptor density (Bmax) determined by inhibition of 0.2 nM [³H] MDL-100,907 binding in mouse frontal cortex.

### DETAILED DESCRIPTION

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

The singular forms “a,” “an” and “the” are intended to include the plural forms as well, unless the context clearly indicates otherwise.

Furthermore, the term “about” as used herein when referring to a measurable value such as a dose, time, temperature, and the like, is meant to encompass variations of ±20%, ±10%, ±5%, ±1%, ±0.5%, or even ±0.1% of the specified amount.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Unless the context indicates otherwise, it is specifically intended that the various features described herein can be used in any combination.

As used herein, the terms “reduce,” “decrease,” “lessen” and similar terms mean a decrease of at least about 10%, about 15%, about 20%, about 25%, about 35%, about 50%, about 75%, about 80%, about 85%, about 90%, about 95%, about 97%, or more.

As used herein, the terms “improve,” “increase,” “enhance,” and similar terms indicate an increase of at least about 10%, about 15%, about 20%, about 25%, about 50%, about 75%, about 100%, about 150%, about 200%, about 300%, about 400%, about 500%, or more.

Reference to a particular numerical value includes at least that particular value unless the context clearly dictates otherwise. When a range of values is expressed, another embodiment includes from the one particular value and/or to the other particular value. Further, reference to values stated in ranges include each and every value within that range. All ranges are inclusive and combinable.

As used herein, “substantially absent” with reference to XRPD diffractogram peak means the peak has a relative intensity compared to a reference peak present in the diffractogram of less than about 5%, less than about 4%, less than about 3%, less than about 2%, or less than about 1% of the intensity of the reference peak, or that the peak is not detectable.

XRPD diffractograms and XRPD peak positions may be acquired using Cu Kα radiation.

DSC thermograms and TGA thermograms may be acquired using a heating rate of 20° C./min.

All disease and disorders listed herein are defined as described in the Diagnostic and Statistical Manual of Mental Disorders (DSM-5), published by the American Psychiatric Association.

As used herein, “treating” and like terms refer to reducing the severity and/or frequency of one or more symptoms, eliminating one or more symptoms and/or the underlying cause of said symptoms, reducing the frequency or likelihood of one or more symptoms and/or their underlying cause, delaying, preventing and/or slowing the progression of diseases and/or disorders and improving or remediating damage caused, directly or indirectly, by the diseases and/or disorders.

As used herein, “therapeutically-effective dose” means a dose sufficient to achieve the intended therapeutic purpose, such as, to alleviate a sign or symptom of a disease or disorder in a patient.

As used herein, “psychotherapy support” as used herein includes, but is not limited to, any suitable psychotherapy techniques used in clinical practice. (Duncan, Hubble & Miller, 2004). In general, psychotherapy refers to the treatment of mental or emotional disorder or of related symptoms by psychological means, rather than medical, physical, or pharmaceutical means.

The term “titration period” refers to the length of time between administration of a maintenance dose of AD (e.g., SSRI) and/or benzodiazepine and administration of a starting dose of AD (e.g., SSRI) and/or benzodiazepine or cessation of AD (e.g., SSRI) and/or benzodiazepine. The term “AD titration period” refers to the length of time between the administration of a patient's maintenance dose of AD and patient's lowest dose of AD or the cessation of AD. Day 1 of the AD titration period (ADTP) refers to the day after the final administration of the patient's maintenance dose of AD. The term “SSRI titration period” refers to the length of time between the administration of a patient's maintenance dose of SSRI and patient's lowest dose of SSRI or the cessation of SSRI. Day 1 of the SSRI titration period (STP) refers to the day after the final administration of the patient's maintenance dose of SSRI. Day 1 of the benzodiazepine titration period (BTP) refers to the first day of administration of benzodiazepine. In some embodiments, Day 1 of the AD (e.g., SSRI) Titration Period (ADTP Day 1) is the first day of a titrated (lower) dose of AD or no AD, and Day 1 of the benzo titration period (BTP Day 1) is the first day of benzodiazepine administration. In some embodiments, BTP Day 1 and ADTP Day 1 are the same day. In some embodiments BTP Day 1 is before ADTP Day 1. In some embodiments, BTP Day 1 is after ADTP Day 1.

The term “maintenance period” refers to a time period in which a patient is administered a maintenance dose of a therapeutic, for example, a maintenance dose of a benzodiazepine or AD (e.g., SSRI). For example, an AD (e.g., SSRI) maintenance period refers to a time period in which a patient is administered a maintenance dose of an AD (e.g., SSRI).

The term “starting dose” refers to the initial dose of benzodiazepine or SSRI administered to a patient.

The term “maintenance dose” refers to a dose of benzodiazepine or AD (e.g., SSRI) that a patient is administered on a continuous basis (e.g., 2 weeks or more).

The term “reduced dose” refers to a dose of AD (e.g., SSRI) or benzodiazepine that is less than the maintenance dose.

### Psilocybin

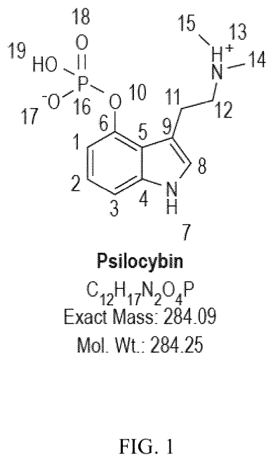

The compositions and methods provided herein comprise psilocybin. A numbered structural formula of psilocybin is shown in FIG. 1. Novel polymorphs and hydrates of psilocybin, along with the preparation and formulations thereof are disclosed in PCT/IB2018/057811 (published as WO2019/073379), which is incorporated by reference herein in its entirety. PCT/IB2018/057811 discloses a number of formulations and the challenges of formulating psilocybin due to e.g., its hygroscopicity and poor flow characteristics. PCT/IB2018/057811 also discloses the importance of a controlled aqueous crystallization process. Further details about the synthesis, characterization, and formulation of psilocybin are provided in PCT/IB2018/057811.

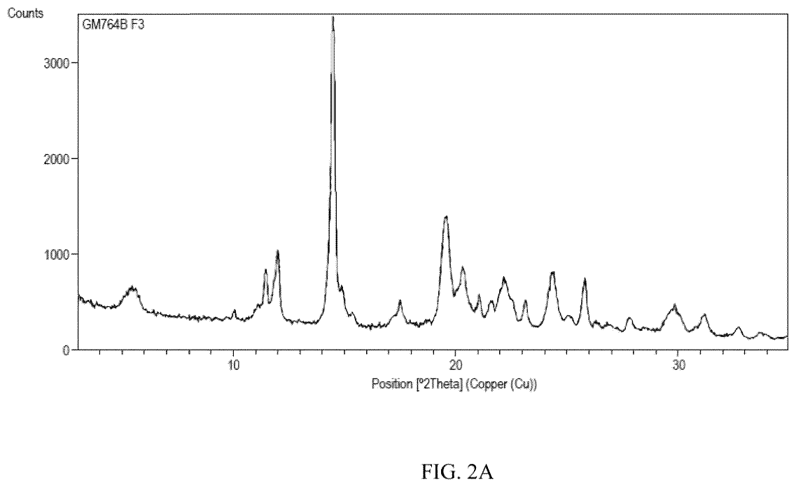

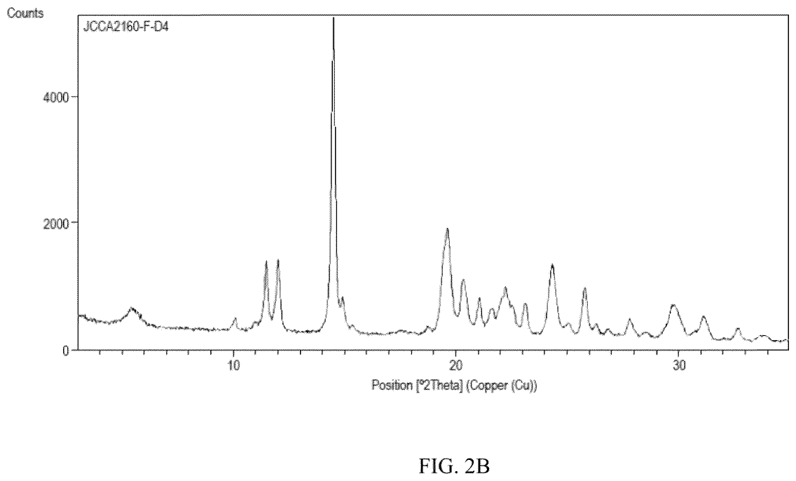

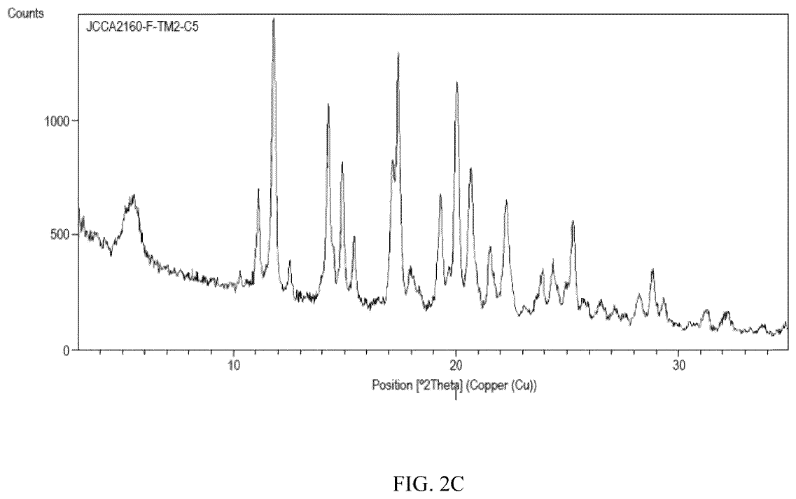

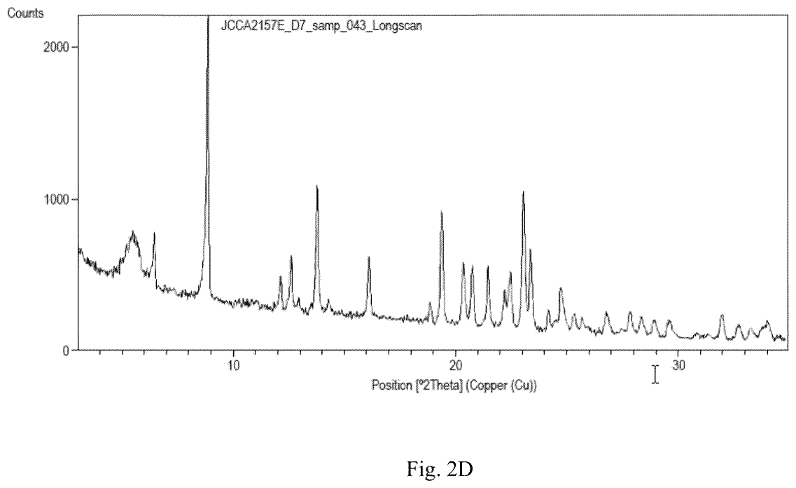

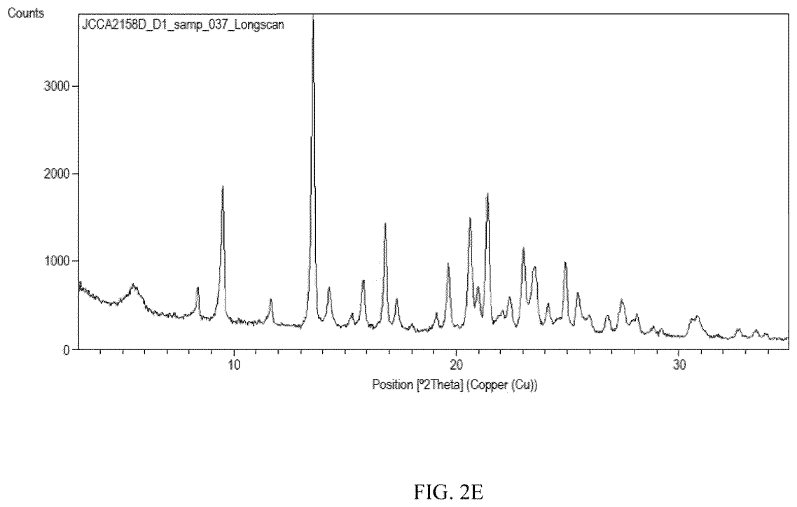

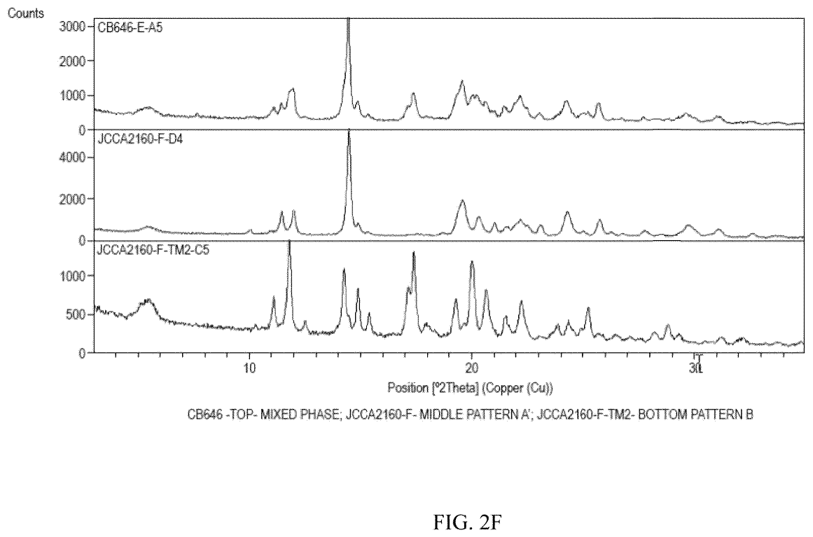

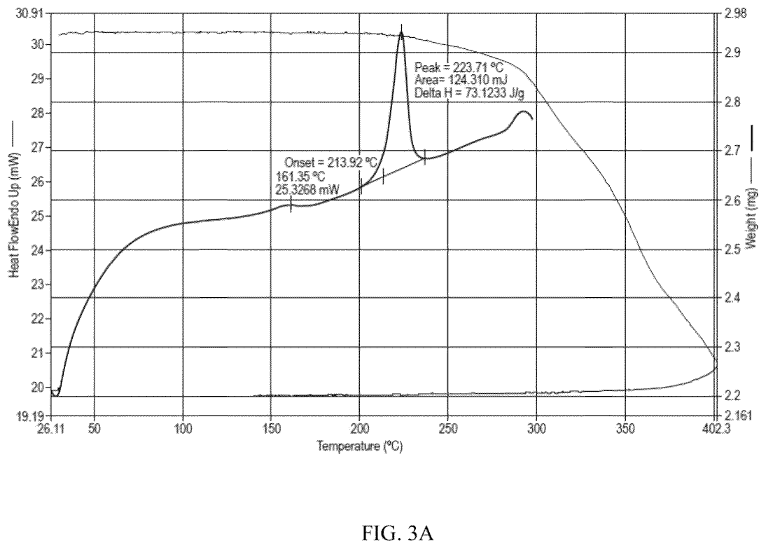

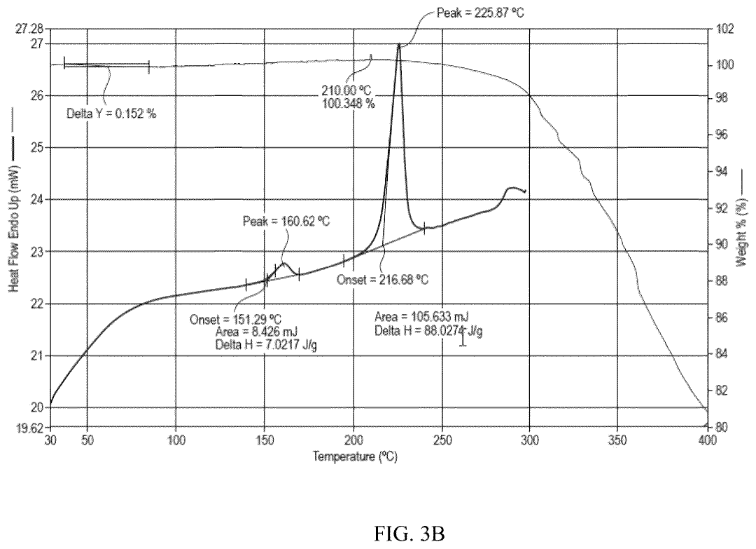

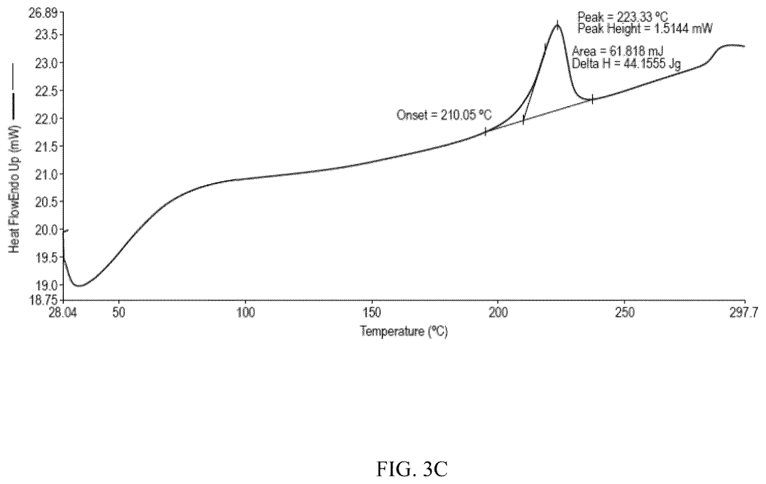

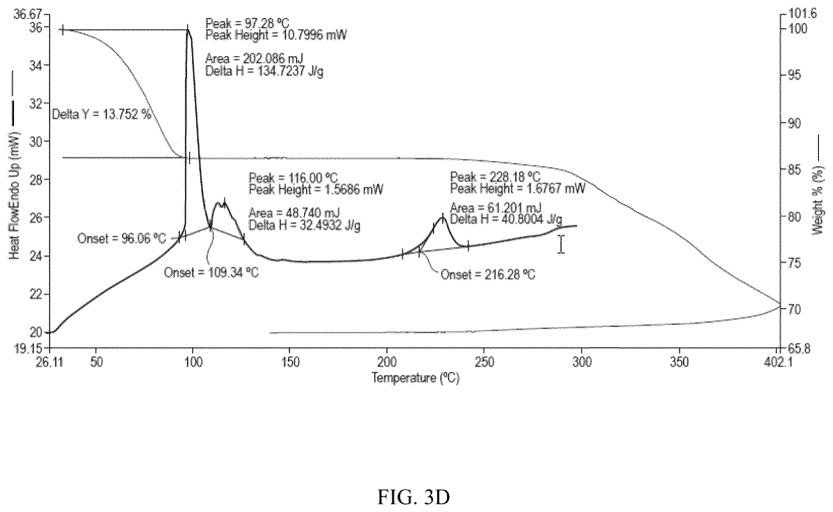

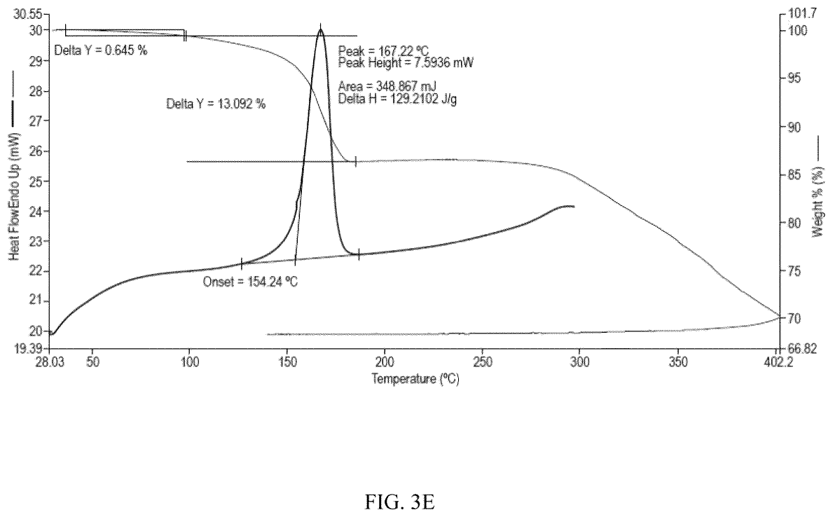

In some embodiments, the psilocybin comprises crystalline psilocybin in the form Polymorph A or Polymorph A′, as described herein, the crystalline psilocybin exhibiting peaks in an XRPD diffractogram at 11.5, 12.0 and 14.5° 2θ±0.1°2θ. In some embodiments, the crystalline psilocybin further exhibits at least one peak in the XRPD diffractogram at 19.7, 20.4, 22.2, 24.3 or 25.7 °2θ±0.1°2θ. Illustrative XRPD diffractograms are provided as FIG. 2A and FIG. 2B. In some embodiments, the crystalline psilocybin exhibits an endothermic event in a DSC thermogram having a first onset temperature of between 145° C. and 165° C. and a second onset temperature of between 205° C. and 220° C. Illustrative DSC thermograms are provided as FIG. 3A and FIG. 3B.

### Polymorph A

The present disclosure provides crystalline psilocybin in the form Polymorph A, characterized by one or more of:
(a) peaks in an XRPD diffractogram at 11.5, 12.0, 14.5, and 17.5, 2θ±0.1°2θ;
(b) peaks in an XRPD diffractogram at 11.5, 12.0, 14.5 and 17.5, 2θ±0.1° 2θ, further characterized by at least one further peak at 19.7, 20.4, 22.2, 24.3 or 25.7° 2θ±0.1° 2θ;
(c) an XRPD diffractogram as substantially illustrated in FIG. 2A; or
(d) an endothermic event in a DSC thermogram having an endothermic event in a DSC thermogram having a first onset temperature of between 145° C. and 165° C. and a second onset temperature of between 205° C. and 220° C. substantially as illustrated in FIG. 3A.

In some embodiments, the peak at 17.5° 2θ±0.1°2θ has a relative intensity compared to the peak at 14.5° 2θ±0.1° 2θ of at least 5%, at least 6%, at least 7%, at least 8%, at least 9%, or at least 10%.

In some embodiments, the crystalline psilocybin of Polymorph A exhibits an XRPD diffractogram having at least 3, 4, 5, 6, 7, 8, 9, 10, 11, 12, 13, 14, 15, 16, or 17 of the peaks listed in Table 1, or equivalent peaks within about ±0.1°2θ of the peaks listed in Table 1. Polymorph A exhibits a peak at 17.5, 2θ±0.1° 2θ that is substantially absent in Polymorph A′.

TABLE 1
XRPD peak positions for Polymorph A

| Position | Relative Intensity |
|---|---|
| [°2Th.] | [%] |
| 5.6 | 8.42 |
| 11.5 | 13.05 |
| 12.0 | 26.45 |
| 14.5 | 100 |
| 17.5 | 10.71 |
| 19.7 | 37.29 |
| 20.4 | 20.06 |
| 22.2 | 17.83 |
| 23.2 | 6.99 |
| 24.3 | 17.93 |
| 25.7 | 16.4 |
| 26.8 | 3.15 |
| 27.8 | 4.54 |
| 29.7 | 9.53 |
| 31.2 | 6.51 |
| 32.6 | 2.45 |
| 33.7 | 1.75 |

In some embodiments, crystalline psilocybin Polymorph A exhibits XRPD diffractogram peaks at 11.5, 12.0, 14.5, and 17.5° 2θ±0.1°2θ. In some embodiments, crystalline psilocybin Polymorph A exhibits at least one additional peak appearing at 19.7, 20.4, 22.2, 24.3 or 25.7° 2θ±0.1° 2θ. In some embodiments, crystalline psilocybin Polymorph A exhibits at least two additional peaks appearing at 19.7, 20.4, 22.2, 24.3 or 25.7° 2θ±0.1° 2θ. In some embodiments, crystalline psilocybin Polymorph A exhibits at least three additional peaks appearing at 19.7, 20.4, 22.2, 24.3 or 25.7 °2θ±0.1°2θ. In some embodiments, crystalline psilocybin Polymorph A exhibits an XRPD diffractogram substantially the same as the XRPD diffractogram shown in FIG. 2A.

In some embodiments, crystalline psilocybin Polymorph A is characterized by XRPD diffractogram peaks at 14.5 and 17.5° 2θ±0.1° 2θ with the peak at 17.5° 2θ having an intensity which is at least about 5%, at least about 6%, at least about 7%, at least about 8%, at least about 9%, or at least about 10% of the intensity of the peak at 14.5° 2θ.

In some embodiments, the crystalline psilocybin Polymorph A exhibits no peak at 10.1—that is, the peak at 10.1 is absent or substantially absent. In some embodiments, the crystalline psilocybin Polymorph A is characterized by an XRPD diffractogram that is substantially absent peaks at 8.9±0.1, 12.6±0.1 and 13.8±0.1 °2θ. In some embodiments, the Polymorph A is characterized by peaks in an XRPD diffractogram at 11.5±0.1, 12.0±0.1, 14.5±0.1 and 17.5±0.1° 2θ, wherein the peak at 17.5±0.1° 2θ has a relative intensity compared to the peak at 14.5±0.1 °2θ of at least 5%, and wherein the XRPD diffractogram is substantially absent a peak at 10.1±0.1° 2θ.

In some embodiments, crystalline psilocybin Polymorph A is characterized by an endothermic event in a DSC thermogram having a first onset temperature of between 145° C. and 165° C. such as between 145 and 160° C., or such as between 145 and 155° C. and a second onset temperature of between 205 and 220° C., such as between 21° and 220° C., such as between 21° and 218° C., or such as between 21° and 216° C. In some embodiments, crystalline psilocybin Polymorph A exhibits an endothermic event in a DSC thermogram having an onset temperature of between about 205 and about 220° C., between about 210 and about 220° C., between about 210 and about 218° C., or between about 210 and about 216° C. In some embodiments, crystalline psilocybin Polymorph A further exhibits an endothermic event in the DSC thermogram having an onset temperature of between about 145 and about 165° C., between about 145 and about 160° C., or between about 145 and about 155° C. In some embodiments, crystalline psilocybin Polymorph A exhibits an endothermic event having an onset temperature of between about 205 and about 220° C., between about 210 and about 220° C., between about 210 and about 218° C., or between about 210 and about 216° C.; and an endothermic event having an onset temperature of between about 145 and about 165° C., between about 145 and about 160° C., between about 145 and about 155° C., in a DSC thermogram. In some embodiments, crystalline psilocybin Polymorph A exhibits a DSC thermogram substantially the same as the DSC thermogram in FIG. 3A.

In some embodiments, crystalline psilocybin Polymorph A exhibits a DSC thermogram that is substantially absent an endothermic event having an onset temperature of between 85° C. and 105° C.

In some embodiments, crystalline psilocybin Polymorph A exhibits a water content of <0.5% w/w, <0.4% w/w, <0.3% w/w, <0.2% w/w, or <0.1% w/w. The water content of a crystalline compound can be determined by known methods, for example Karl Fischer Titration. In some embodiments, crystalline psilocybin Polymorph A exhibits <0.5% w/w loss, <0.4% w/w, <0.3% w/w, <0.2% w/w, or <0.1% w/w in the TGA thermogram between ambient temperature, e.g., about 25° C., and 200° C. In some embodiments, crystalline psilocybin Polymorph A loses less than 2% by weight, less than 1% by weight, or than 0.5% by weight in a loss on drying test, e.g., a loss on drying test performed at 70° C.

In some embodiments, crystalline psilocybin Polymorph A is a highly pure crystalline form of Polymorph A, for example, the in a loss on drying test psilocybin comprises at least 90%, at least 95%, at least 99%, or at least 99.5% by weight crystalline psilocybin of Polymorph A.

In some embodiments, crystalline psilocybin Polymorph A is a white to off white solid.

In some embodiments, crystalline psilocybin Polymorph A is chemically pure, for example the psilocybin has a chemical purity of greater than 97%, 98%, or 99% by HPLC. In some embodiments, crystalline psilocybin Polymorph A has no single impurity of greater than 1%, greater than 0.5%, greater than 0.4%, greater than 0.3%, or greater than 0.2% e.g., the impurity phosphoric acid as measured by 31P NMR, or the impurity psilocin measured by HPLC. In some embodiments, crystalline psilocybin Polymorph A has a chemical purity of greater than 97 area %, greater than 98 area %, or greater than 99 area % by HPLC. In some embodiments, crystalline psilocybin Polymorph A has no single impurity greater than 1 area %, greater than 0.5 area %, greater than 0.4%, greater than 0.3%, or greater than 0.2% as measured by HPLC. In some embodiments, crystalline psilocybin Polymorph A does not contain psilocin at a level greater than 1 area %, greater than 0.5 area %, greater than 0.4%, greater than 0.3%, or greater than 0.2% as measured by HPLC. In some embodiments, crystalline psilocybin Polymorph A does not contain phosphoric acid at a level greater than 1 weight %, greater than 0.5 weight %, greater than 0.4 weight %, 0.3 weight %, or greater than 0.2 weight %, as measured by 31P NMR. In some embodiments, crystalline psilocybin Polymorph A has a chemical assay of at least 95 weight %, at least 96 weight %, or at least 98 weight %. In some embodiments, the crystalline psilocybin Polymorph A is characterized by a chemical purity of greater than 97% by HPLC crystalline psilocybin Polymorph A. In some embodiments, the crystalline psilocybin Polymorph A is characterized by a chemical purity of greater than 97% by HPLC crystalline psilocybin Polymorph A and by having no single impurity of greater than 1% including phosphoric acid as measured by 31P NMR, and psilocin as measured by HPLC.

### Polymorph A′

The present disclosure provides crystalline psilocybin in the form of Polymorph A′, characterized by one or more of
(a) peaks in an XRPD diffractogram at 11.5, 12.0 and 14.5° 2θ±0.1°2θ, but absent or substantially absent of a peak at 17.5° 2θ±0.1° 2θ;
(b) peaks in an XRPD diffractogram at 11.5, 12.0 and 14.5° 2θ±0.1°2θ, but absent or substantially absent of a peak at 17.5° 2θ±0.1°2θ, further characterized by at least one further peak at 19.7, 20.4, 22.2, 24.3 or 25.7 °2θ±0.1° 2θ;
(c) an XRPD diffractogram as substantially illustrated in FIG. 2B; or
(d) an endothermic event in a DSC thermogram having a first onset temperature of between 145° C. and 165° C. and a second onset temperature of between 205° C. and 220° C. substantially as illustrated in FIG. 3B.

In some embodiments, the crystalline psilocybin comprises crystalline psilocybin Polymorph A′. Crystalline psilocybin Polymorph A′ exhibits peaks in an XRPD diffractogram at 11.5, 12.0 and 14.5° 2θ±0.1°2θ, but absent or substantially absent of a peak at 17.5° 2θ±0.1° 2θ. In some embodiments, crystalline psilocybin Polymorph A′ further exhibits 1, 2, 3, 4, or 5 peaks selected from 19.7, 20.4, 22.2, 24.3 or 25.7 °2θ±0.1°2θ. An illustrative XRPD diffractogram for Polymorph A′ is provided as FIG. 2B. An illustrative DSC thermogram having an onset temperature of between 205 and 220° C. for Polymorph A′ is provided as FIG. 3B.

In some embodiments, psilocybin Polymorph A′ exhibits an XRPD diffractogram as summarized in Table 2. In some embodiments, crystalline psilocybin Polymorph A′ exhibits at least 3, 4, 5, 6, 7, 8, 9, 10, 11, 12, 13, 14, 15, 16, 17, 18, 19, 20, 21, 22, 23, 24, or 25 peaks listed of Table 2 or equivalent peaks within about +0.1° 2θ, and absent or substantially absent peak at 17.5° 2θ±0.1° 2θ.

TABLE 2
XRPD peak positions for Polymorph A′

| Position | Relative Intensity |
|---|---|
| [°2Th.] | [%] |
| 5.5 | 4.89 |
| 10.1 | 4.09 |
| 11.5 | 22.05 |
| 12.0 | 22.77 |
| 14.5 | 100 |
| 14.9 | 11.29 |
| 17.5 | 1.08 |
| 18.7 | 2.44 |
| 19.4 | 23.02 |
| 19.6 | 33.7 |
| 20.3 | 17.01 |
| 21.1 | 12.08 |
| 21.6 | 8.51 |
| 22.2 | 15.54 |
| 22.6 | 8.78 |
| 23.1 | 10.11 |
| 24.3 | 21.83 |
| 25.1 | 4.36 |
| 25.8 | 15.4 |
| 26.3 | 4.28 |
| 26.8 | 2.86 |
| 27.8 | 5.96 |
| 28.6 | 1.91 |
| 29.7 | 10.56 |
| 31.1 | 7.35 |
| 32.6 | 3.72 |
| 33.8 | 1.54 |

In some embodiments, crystalline psilocybin Polymorph A′ exhibits XRPD diffractogram peaks at 11.5, 12.0, and 14.5° 2θ±0.1° 2θ but substantially absent of a peak at 17.5° 2θ±0.1° 2θ. In some embodiments, crystalline psilocybin Polymorph A′ further exhibits at least one additional peak appearing at 19.7, 20.4, 22.2, 24.3, or 25.7 °2θ±0.1° 2θ. In some embodiments, crystalline psilocybin Polymorph A′ exhibits at least two additional peaks appearing at 19.7, 20.4, 22.2, 24.3, or 25.7 °2θ±0.1°2θ. In some embodiments, crystalline psilocybin Polymorph A′ exhibits and is distinguished from Polymorph A by the presence of a peak appearing at 10.1 °2θ±0.1°2θ. In yet a further embodiment, crystalline psilocybin Polymorph A′ exhibits an XRPD diffractogram substantially the same as the XRPD diffractogram shown in FIG. 2B.

In some embodiments, crystalline psilocybin Polymorph A′ exhibits XRPD diffractogram peaks at 14.5 and 17.5° 2θ±0.1°2θ, wherein the intensity of the peak at 17.5° 2θ is less than 5%, less than 4%, less than 3%, less than 2%, or less than 1% of the intensity of the peak at 14.5° 2θ.

In some embodiments, crystalline psilocybin Polymorph A′ exhibits XRPD diffractogram peaks at 10.1 and 14.5° 2θ±0.1° 2θ, wherein the intensity of the peak at 10.1°2θ is at least 1%, at least 2%, at least 3%, or at least 4% of the intensity of the peak at 14.5° 2θ.

In some embodiments, crystalline psilocybin Polymorph A′ is characterized by an endothermic event in a DSC thermogram having a first onset temperature of between 145° C. and 165° C. such as between 145 and 160° C., or such as between 145 and 155° C. and a second onset temperature of between 205 and 220° C., such as between 21° and 220° C., such as between 21° and 218° C., or such as between 21° and 216° C. In some embodiments, crystalline psilocybin Polymorph A′ is characterized by an endothermic event in a DSC thermogram having an onset temperature of between about 205 and about 220° C., between about 210 and about 220° C., between about 210 and about 218° C., or between about 210 and about 216° C. In some embodiments, crystalline psilocybin Polymorph A′ exhibits an endothermic event in the DSC thermogram having an onset temperature of between about 145 and about 165° C., between about 145 and about 160° C., or between about 145 and about 155° C. In some embodiments, crystalline psilocybin Polymorph A′ exhibits an endothermic event having an onset temperature of between about 205 and about 220° C., between about 210 and about 220° C., between about 210 and about 218° C., or between about 210 and about 216° C., and an endothermic event having an onset temperature of between about 145 and about 165° C., between about 145 and about 160° C., or between about 145 and about 155° C., in a DSC thermogram. In some embodiments, crystalline psilocybin Polymorph A′ exhibits a DSC thermogram substantially the same as the DSC thermogram in FIG. 3B.

In some embodiments, crystalline psilocybin Polymorph A′ exhibits a water content of <0.5% w/w, <0.4% w/w, <0.3% w/w, <0.2% w/w, or <0.1% w/w. Methods to determine the water content of a crystalline compound are known, for example Karl Fischer Titration. In some embodiments, crystalline psilocybin Polymorph A′ exhibits <0.5% w/w loss, <0.4% w/w, <0.3% w/w, <0.2% w/w, <0.1% w/w in the TGA thermogram between ambient temperature, e.g., 25° C., and 200° C. In some embodiments, crystalline psilocybin Polymorph A′ loses less than 2% by weight, less than 1% by weight, or less than 0.5% by weight in a loss on drying test. In some embodiments, the loss on drying test is performed at 70° C.

In some embodiments, crystalline psilocybin Polymorph A′ is a highly pure crystalline form of Polymorph A′. In some embodiments, the crystalline psilocybin comprises at least 90%, 95%, 99%, or 99.5% by weight of Polymorph A′.

In some embodiments, crystalline psilocybin Polymorph A's is a white to off white solid.

In some embodiments, crystalline psilocybin Polymorph A′ is chemically pure, for example the psilocybin has a chemical purity of greater than 97%, greater than 98%, or than 99% by HPLC. In some embodiments, crystalline psilocybin Polymorph A′ has no single impurity of greater than 1% or greater than 0.5%, e.g., the impurity phosphoric acid as measured by 31P NMR or the impurity psilocin as measured by HPLC. In some embodiments, crystalline psilocybin Polymorph A′ has a chemical purity of greater than 97 area %, greater than 98 area %, or greater than 99 area % by HPLC. In some embodiments, crystalline psilocybin Polymorph A′ has no single impurity greater than 1 area % or greater than 0.5 area %, e.g., as measured by HPLC. In some embodiments, crystalline psilocybin Polymorph A′ does not contain psilocin at a level greater than 1 area % or greater than 0.5 area % as measured by HPLC. In some embodiments, crystalline psilocybin Polymorph A′ does not contain phosphoric acid at a level greater than 1 weight % or greater than 0.5 weight % as measured by 31P NMR. In some embodiments, crystalline psilocybin Polymorph A′ has a chemical assay of at least 95 weight %, at least 96 weight %, or at least 98 weight %.

In some embodiments, crystalline psilocybin Polymorph A′ is chemically pure, for example the psilocybin has a chemical purity of greater than 97%, 98%, or 99% by HPLC. In some embodiments, crystalline psilocybin Polymorph A′ has no single impurity of greater than 1%, greater than 0.5%, greater than 0.4%, greater than 0.3%, or greater than 0.2% e.g., the impurity phosphoric acid as measured by 31P NMR, or the impurity psilocin measured by HPLC. In some embodiments, crystalline psilocybin Polymorph A′ has a chemical purity of greater than 97 area %, greater than 98 area %, or greater than 99 area % by HPLC. In some embodiments, crystalline psilocybin Polymorph A′ has no single impurity greater than 1 area %, greater than 0.5 area %, greater than 0.4%, greater than 0.3%, or greater than 0.2% as measured by HPLC. In some embodiments, crystalline psilocybin Polymorph A′ does not contain psilocin at a level greater than 1 area %, greater than 0.5 area %, greater than 0.4%, greater than 0.3%, or greater than 0.2% as measured by HPLC. In some embodiments, crystalline psilocybin Polymorph A′ does not contain phosphoric acid at a level greater than 1 weight %, greater than 0.5 weight %, greater than 0.4 weight %, 0.3 weight %, or greater than 0.2 weight %, as measured by 31P NMR. In some embodiments, crystalline psilocybin Polymorph A′ has a chemical assay of at least 95 weight %, at least 96 weight %, or at least 98 weight %.

Illustrative XRPD diffractograms for high purity crystalline psilocybin, Polymorph A or Polymorph A′ are provided in FIG. 2A and FIG. 2B. Illustrative DSC thermographs for high purity crystalline psilocybin, Polymorph A or Polymorph A′ are provided in FIG. 2A and FIG. 2B.

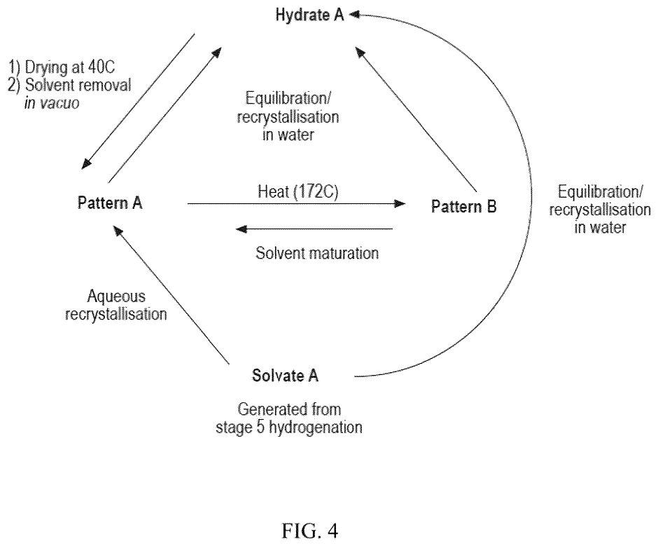

Polymorph A (including its isostructural variant Polymorph A′) (FIG. 2A and FIG. 2B) differs from Polymorph B (FIG. 2C), the Hydrate A (FIG. 2D) and the ethanol solvate (FIG. 2E: Solvate A), and the relationship between some of the different forms is illustrated in FIG. 4.

In some embodiments, the crystalline psilocybin Polymorph A or Polymorph A′ is a white to off white solid, and/or has a chemical purity of greater than 97%, 98%, or 99% by HPLC. In some embodiments, crystalline psilocybin Polymorph A or Polymorph A′ has no single impurity of greater than 1%, greater than 0.5%, greater than 0.4%, greater than 0.3%, or greater than 0.2% e.g., the impurity phosphoric acid as measured by ³¹P NMR, or the impurity psilocin measured by HPLC. In some embodiments, crystalline psilocybin Polymorph A or Polymorph A′ has a chemical purity of greater than 97 area %, greater than 98 area %, or greater than 99 area % by HPLC. In some embodiments, crystalline psilocybin Polymorph A or Polymorph A′ has no single impurity greater than 1 area %, greater than 0.5 area %, greater than 0.4%, greater than 0.3%, or greater than 0.2% as measured by HPLC. In some embodiments, crystalline psilocybin Polymorph A or Polymorph A′ does not contain psilocin at a level greater than 1 area %, greater than 0.5 area %, greater than 0.4%, greater than 0.3%, or greater than 0.2% as measured by HPLC. In some embodiments, crystalline psilocybin Polymorph A or Polymorph A′ does not contain phosphoric acid at a level greater than 1 weight %, greater than 0.5 weight %, greater than 0.4 weight %, 0.3 weight %, or greater than 0.2 weight %, as measured by ³¹P NMR. In some embodiments, crystalline psilocybin Polymorph A or Polymorph A′ has a chemical assay of at least 95 weight %, at least 96 weight %, or at least 98 weight %.

The heating of Polymorph A or A′ results in an endothermic event having an onset temperature of circa 150° C. corresponding to solid-solid transition of Polymorph A or Polymorph A′ to Polymorph B. Continued heating of the resulting solid, i.e., Polymorph B, results in a second endothermic event corresponding to a melting point having an onset temperature of between 205 and 220° C. (see FIG. 3A and FIG. 3B).

### Hydrate A

In some embodiments, the disclosure provides a crystalline form of psilocybin, Hydrate A. In some embodiments, crystalline psilocybin Hydrate A exhibits peaks in an XRPD diffractogram at 8.9, 12.6 and 13.8°2θ±0.1°2θ. In some embodiments, crystalline psilocybin Hydrate A further exhibits at least 1, 2, 3, 4, or 5 further peaks at 6.5, 12.2, 19.4, 20.4 or 20.8°2θ±0.1°2θ. An illustrative XRPD diffractogram is provided as FIG. 2D. In some embodiments, crystalline psilocybin Hydrate A further exhibits an endothermic event in a DSC thermogram having a first onset temperature of between 90° C. and 100° C., a second onset temperature of between 100° C. and 120° C. and a third onset temperature of between 210° C. and 220° C. An illustrative DSC thermogram is provided as FIG. 2D.

In some embodiments, psilocybin Hydrate A exhibits an XRPD diffractogram comprising at least 3, 4, 5, 6, 7, 8, 9, or 10 peaks listed in Table 3 or equivalent peaks within about +0.1° 2θ.

TABLE 3
XRPD peak positions for Hydrate A

| Position | Relative Intensity |
|---|---|
| [°2Th.] | [%] |
| 5.6 | 14.4 |
| 6.5 | 18.84 |
| 8.9 | 100 |
| 12.2 | 11.51 |
| 12.6 | 18.65 |
| 13.8 | 44.22 |
| 16.2 | 21.22 |
| 18.9 | 6.62 |
| 19.4 | 38.68 |
| 20.4 | 21.32 |
| 20.8 | 19.73 |
| 21.5 | 20.75 |
| 22.3 | 12.8 |
| 22.5 | 19.38 |
| 23.1 | 47.53 |
| 23.5 | 25.79 |
| 24.3 | 5.62 |
| 24.8 | 14.62 |
| 25.4 | 5.27 |
| 26.9 | 6.53 |
| 27.9 | 7.82 |
| 28.4 | 5.78 |
| 29.0 | 5.09 |
| 29.7 | 4.83 |
| 32.1 | 8.27 |
| 32.8 | 4.81 |
| 33.4 | 3.74 |
| 34.2 | 5.96 |

In some embodiments, crystalline psilocybin Hydrate A exhibits XRPD diffractogram peaks at 8.9, 12.6 and 13.8°2θ±0.1°2θ. In some embodiments, crystalline psilocybin Hydrate A exhibits at least one peak appearing at 6.5, 12.2, 19.4, 20.4 or 20.8° 2θ±0.1°2θ. In some embodiments, crystalline psilocybin Hydrate A exhibits at least two peaks appearing at 6.5, 12.2, 19.4, 20.4 or 20.8° 2θ±0.1°2θ. In some embodiments, crystalline psilocybin Hydrate A exhibits an XRPD diffractogram substantially the same as the XRPD diffractogram shown in FIG. 2D. In some embodiments, crystalline Hydrate A is characterized by XRPD peaks at 8.9±0.1, 13.8±0.1, 19.4±0.1, 23.1±0.1 and 23.5±0.1°2θ. In some embodiments, crystalline Hydrate A is further characterized by XRPD peaks at 6.5±0.1, 12.6±0.1, 16.2±0.1, 20.4±0.1, 20.8±0.1, 21.5±0.1 and 22.5±0.1° 2θ. In some embodiments, crystalline Hydrate A is further characterized by XRPD peaks at 5.6±0.1, 12.2±0.1, 22.3±0.1 and 24.8±0.1° 2θ.

In certain embodiments, crystalline psilocybin Hydrate A is characterized by an endothermic event in a DSC thermogram having a first onset temperature of between 85° C. and 105° C., such as between 90° C. and 100° C. and most preferably at about 96° C., a second onset temperature of between 100° C. and 120° C. such as between 105° C. and 115° C., and most preferably at about 109° C. and a third onset temperature of between 205 and 220° C., such as between 21° and 220° C., such as between 21° and 218° C., or such as between 21° and 216° C., or about 216° C. In some embodiments, crystalline psilocybin Hydrate A exhibits an endothermic event in a DSC thermogram having an onset temperature of between about 205 and about 220° C., between about 210 and about 220° C., between about 210 and about 218° C., or between about 210 and about 216° C. In some embodiments, crystalline psilocybin Hydrate A exhibits an endothermic event in the DSC thermogram having an onset temperature of between about 85 and about 105° C., or between about 90 and about 100° C. In some embodiments, crystalline psilocybin Hydrate A exhibits an endothermic event having an onset temperature of between about 205 and about 220° C., between about 210 and about 220° C., between about 210 and about 218° C., or between about 210 and about 216° C., and an endothermic event having an onset temperature of between about 85 and about 105° C. or between about 90 and about 100° C., in a DSC thermogram. In some embodiments, crystalline psilocybin Hydrate A exhibits a DSC thermogram substantially the same as the DSC thermogram in FIG. 3D.

In some embodiments, crystalline psilocybin Hydrate A exhibits a water content of between about 10 and about 18%, between about 12 and about 16%, or about 13%. Methods to determine the water content of a crystalline compound are known, for example Karl Fischer Titration. In some embodiments, crystalline psilocybin Hydrate A exhibits a weight loss in the TGA thermogram of between about 10 and about 18%, between about 12 and about 16%, or about 13%, between ambient temperature, about 25° C., and 120° C.

In some embodiments, crystalline psilocybin Hydrate A is chemically pure, for example the psilocybin has a chemical purity of greater than 97%, 98%, or 99% by HPLC. In some embodiments, crystalline psilocybin Hydrate A has no single impurity of greater than 1%, greater than 0.5%, greater than 0.4%, greater than 0.3%, or greater than 0.2% e.g., the impurity phosphoric acid as measured by ³¹P NMR, or the impurity psilocin measured by HPLC. In some embodiments, crystalline psilocybin Hydrate A has a chemical purity of greater than 97 area %, greater than 98 area %, or greater than 99 area % by HPLC. In some embodiments, crystalline psilocybin Hydrate A has no single impurity greater than 1 area %, greater than 0.5 area %, greater than 0.4%, greater than 0.3%, or greater than 0.2% as measured by HPLC. In some embodiments, crystalline psilocybin Hydrate A does not contain psilocin at a level greater than 1 area %, greater than 0.5 area %, greater than 0.4%, greater than 0.3%, or greater than 0.2% as measured by HPLC. In some embodiments, crystalline psilocybin Hydrate A does not contain phosphoric acid at a level greater than 1 weight %, greater than 0.5 weight %, greater than 0.4 weight %, 0.3 weight %, or greater than 0.2 weight %, as measured by ³¹P NMR. In some embodiments, crystalline psilocybin Hydrate A has a chemical assay of at least 95 weight %, at least 96 weight %, or at least 98 weight %. In some embodiments, the crystalline psilocybin Hydrate A is characterized by a chemical purity of greater than 97% by HPLC crystalline psilocybin Hydrate A. In some embodiments, the crystalline psilocybin Hydrate A is characterized by a chemical purity of greater than 97% by HPLC crystalline psilocybin Hydrate A and by having no single impurity of greater than 1% including phosphoric acid as measured by ³¹P NMR, and psilocin as measured by HPLC.

In some embodiments, crystalline psilocybin Hydrate A is a highly pure crystalline form of Hydrate A. In some embodiments, the crystalline psilocybin comprises at least 90%, at least 95%, at least 99%, or at least 99.5% by weight of Hydrate A.

### Polymorph B

In some embodiments, the disclosure provides a crystalline form of psilocybin, Polymorph B. In some embodiments, crystalline psilocybin Polymorph B exhibits peaks in an XRPD diffractogram at 11.1, 11.8 and 14.3°2θ±0.1°2θ. In some embodiments, crystalline psilocybin Polymorph B exhibits at least 1, 2, 3, 4 or 5 peaks in an XRPD diffractogram at 14.9, 15.4, 19.3, 20.0 or 20.6° 2θ±0.1°2θ. An illustrative XRPD diffractogram of crystalline psilocybin Polymorph B is provided as FIG. 2C. In some embodiments, crystalline psilocybin Polymorph B exhibits a single endothermic event in a DSC thermogram having an onset temperature of between about 205 and about 220° C. An illustrative DSC thermogram of crystalline psilocybin Polymorph B is provided as FIG. 3C.

In some embodiments, psilocybin Polymorph B exhibits an XRPD diffractogram comprising at least 3, 4, 5, 6, 7, 8, 9, or 10 peaks listed in Table 4 or equivalent peaks within about +0.1° 2θ.

TABLE 4
XRPD peak positions for Polymorph B

| Position | Relative Intensity |
|---|---|
| [°2Th.] | [%] |
| 5.5 | 21.33 |
| 11.1 | 36.91 |
| 11.8 | 100.00 |
| 12.5 | 12.73 |
| 14.3 | 70.23 |
| 14.9 | 50.01 |
| 15.4 | 23.67 |
| 17.1 | 51.58 |
| 17.4 | 91.25 |
| 18.0 | 12.61 |
| 19.3 | 39.33 |
| 20.0 | 76.61 |
| 20.6 | 50.26 |
| 21.5 | 20.77 |
| 22.3 | 40.19 |
| 23.9 | 13.32 |
| 24.3 | 16.03 |
| 25.3 | 32.94 |
| 28.3 | 7.60 |
| 28.9 | 17.89 |
| 29.3 | 8.96 |
| 31.3 | 6.57 |
| 32.2 | 6.90 |
| 33.8 | 2.37 |

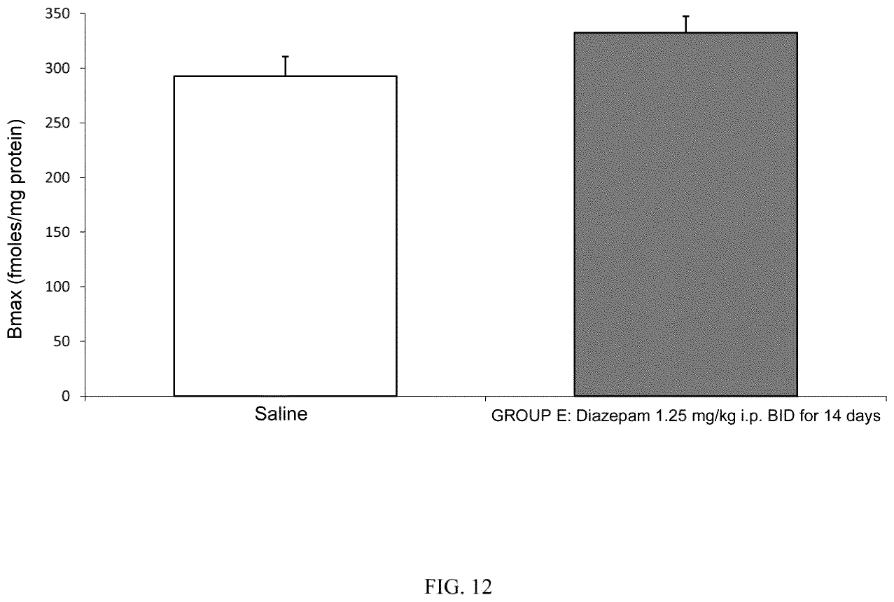

In some embodiments, crystalline psilocybin Polymorph B exhibits XRPD diffractogram peaks at 11.1, 11.8 and 14.3°2θ±0.1°2θ. In some embodiments, crystalline psilocybin Polymorph B exhibits at least one peak at 14.9, 15.4, 19.3, 20.0 or 20.6° 2θ±0.1° 2θ. In some embodiments, crystalline psilocybin Polymorph B exhibits at least two peaks appearing at 14.9, 15.4, 19.3, 20.0 or 20.6° 2θ±0.1°2θ. In some embodiments, crystalline psilocybin Polymorph B exhibits an XRPD diffractogram substantially the same as the XRPD diffractogram shown in FIG. 12C.

In some embodiments, crystalline psilocybin Polymorph B is characterized by a single endothermic event in a DSC thermogram having an onset temperature of between about 205 and about 220° C., between about 210 and about 220° C., between about 210 and about 218° C., or between about 210 and about 216° C. In some embodiments, crystalline psilocybin Polymorph B exhibits a DSC thermogram substantially the same as the DSC thermogram in FIG. 3C.

In some embodiments, crystalline psilocybin Polymorph B exhibits a water content of <0.5% w/w, <0.4% w/w, <0.3% w/w, <0.2% w/w, or <0.1% w/w. Methods to determine the water content of a crystalline compound are known, for example Karl Fischer Titration. In some embodiments, crystalline psilocybin Polymorph B exhibits <0.5% w/w, <0.4% w/w, <0.3% w/w, <0.2% w/w, or <0.1% w/w loss in the TGA thermogram between ambient temperature, about 25° C., and 200° C. In some embodiments, crystalline psilocybin Polymorph B exhibits a loss of less than 2% by weight, less than 1% by weight, or less than 0.5% by weight in a loss on drying test. In some embodiments, the loss on drying test is performed at 70° C.

In some embodiments, crystalline psilocybin Polymorph B is a highly pure crystalline form of Polymorph B, for example, psilocybin comprises at least 90%, at least 95%, at least 99%, or at least 99.5% by weight of Polymorph B.

In some embodiments, crystalline psilocybin Polymorph B is chemically pure, for example the psilocybin has a chemical purity of greater than 97%, 98%, or 99% by HPLC. In some embodiments, crystalline psilocybin Polymorph B has no single impurity of greater than 1%, greater than 0.5%, greater than 0.4%, greater than 0.3%, or greater than 0.2% e.g., the impurity phosphoric acid as measured by ³¹P NMR, or the impurity psilocin measured by HPLC. In some embodiments, crystalline psilocybin Polymorph B has a chemical purity of greater than 97 area %, greater than 98 area %, or greater than 99 area % by HPLC. In some embodiments, crystalline psilocybin Polymorph B has no single impurity greater than 1 area %, greater than 0.5 area %, greater than 0.4%, greater than 0.3%, or greater than 0.2% as measured by HPLC. In some embodiments, crystalline psilocybin Polymorph B does not contain psilocin at a level greater than 1 area %, greater than 0.5 area %, greater than 0.4%, greater than 0.3%, or greater than 0.2% as measured by HPLC. In some embodiments, crystalline psilocybin Polymorph B does not contain phosphoric acid at a level greater than 1 weight %, greater than 0.5 weight %, greater than 0.4 weight %, 0.3 weight %, or greater than 0.2 weight %, as measured by ³¹P NMR. In some embodiments, crystalline psilocybin Polymorph B has a chemical assay of at least 95 weight %, at least 96 weight %, or at least 98 weight %.

In some embodiments, the psilocybin of the disclosure in the form Polymorph A or A′ has the general properties illustrated in Table 5.

TABLE 5

| Appearance: | White to off white solid |
|---|---|
| Major endothermic event in DSC | 210-215° C. |
| (onset temperature) (corresponding | |
| to a melt): | |
| Hygroscopicity: | Psilocybin forms Hydrate A at high |
| | humidity and when added to water |
| | but the water of hydration is lost |
| | rapidly on drying. The anhydrous |
| | form is therefore being developed. |
| Crystalline form: | Anhydrous Polymorph A and/or A′ |
| pKa (calculated): | 1.74, 6.71, 9.75 |
| Solubility | approx. 15 mg/ml in Water |

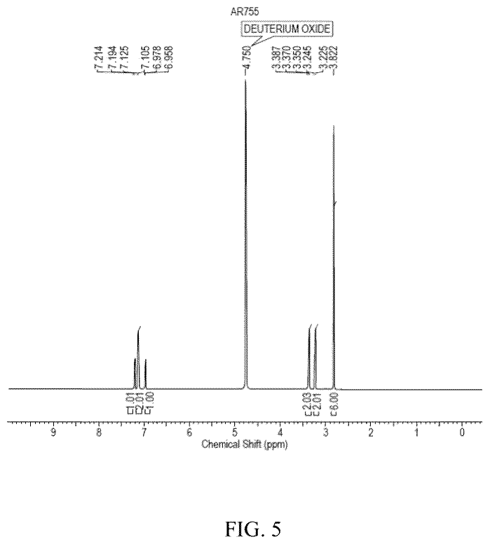

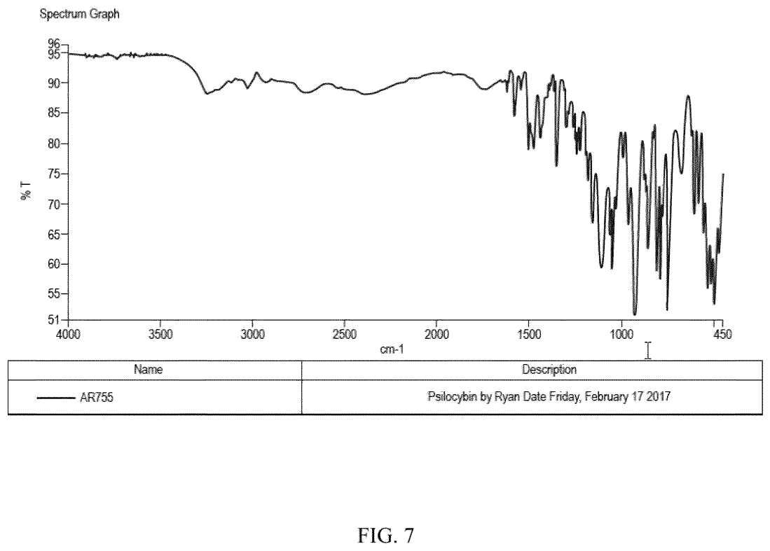

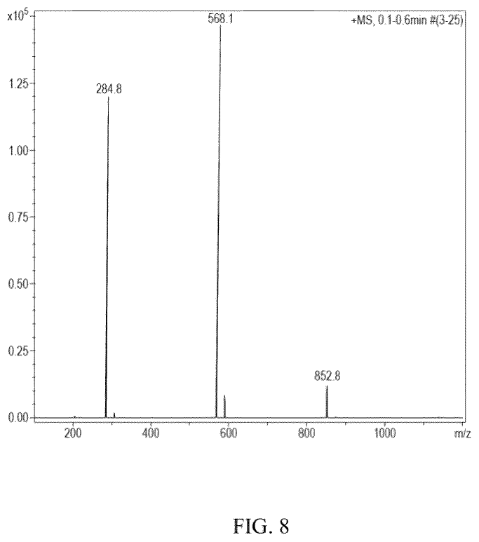

In some embodiments, the psilocybin conforms to the spectra as set out in Table 6 and illustrated in the spectra of FIGS. 5-8.

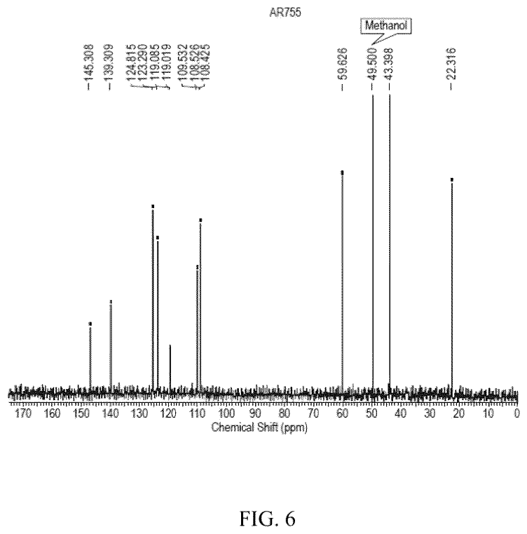

TABLE 6

| Technique | Conclusions |
|---|---|
| Proton (¹H) and Carbon | Assignment of the proton (FIG. 5) and |
| (¹³C) NMR | carbon spectra (FIG. 6) are concordant |
| | with Psilocybin. |
| FT-Infrared Spectroscopy | Assignment of the FT-IR spectrum (FIG. |
| (FT-IR) | 7) is concordant with Psilocybin. |
| Mass Spectroscopy (MS) | Assignment of the mass spectrum (FIG. |
| | 8) is concordant with Psilocybin. |

Alternatively, and independently, the crystalline psilocybin may take the form of Hydrate A or Polymorph B.

In some embodiments, the disclosure provides the crystalline psilocybin in the form Polymorph A or Polymorph A′ for use in medicine. In some embodiments, the disclosure provides crystalline psilocybin Polymorph A for use in medicine. In some embodiments, the disclosure provides crystalline psilocybin Polymorph A′ for use in medicine. In some embodiments, the disclosure provides a high purity crystalline psilocybin Polymorph A for use in medicine. In some embodiments, the disclosure provides a high purity crystalline psilocybin Polymorph A′ for use in medicine. Alternatively, and independently, the crystalline psilocybin may take the form of Hydrate A or Polymorph B.

In some embodiments, the disclosure provides crystalline psilocybin in the form Polymorph A or Polymorph A′ for use in treating a patient in need thereof. Alternatively, and independently, the crystalline psilocybin may take the form of Hydrate A or Polymorph B.

In some embodiments, the disclosure provides crystalline psilocybin, Polymorph A or Polymorph A′, for use in treating a patient in need thereof. In some embodiments, the disclosure provides crystalline psilocybin, Polymorph A or Polymorph A′, for use in treating a patient in need thereof. In some embodiments, the disclosure provides crystalline psilocybin Polymorph A for use in treating a patient in need thereof. In some embodiments, the disclosure provides crystalline psilocybin Polymorph A′ for use in treating a patient in need thereof. In some embodiments, the disclosure provides a high purity crystalline psilocybin Polymorph A for use in treating a patient in need thereof. In some embodiments, the disclosure provides a high purity crystalline psilocybin Polymorph A′ for use in treating a patient in need thereof.

### Pharmaceutical Compositions and Formulations

In some embodiments, the disclosure provides a pharmaceutical composition comprising crystalline psilocybin and one or more pharmaceutically acceptable carriers or excipients.

In some embodiments, the disclosure provides a pharmaceutical formulation comprising high purity psilocybin and one or more pharmaceutically acceptable carriers or excipients. In some embodiments, the disclosure provides a pharmaceutical formulation comprising crystalline psilocybin Polymorph A and one or more pharmaceutically acceptable carriers or excipients. In some embodiments, the disclosure provides a pharmaceutical formulation comprising crystalline psilocybin Polymorph A′ and one or more pharmaceutically carriers or excipients. In some embodiments, the disclosure provides a pharmaceutical formulation comprising high purity crystalline psilocybin, Polymorph A or Polymorph A′, and one or more pharmaceutically acceptable carriers or excipients. In some embodiments, the disclosure provides a pharmaceutical formulation comprising high purity crystalline psilocybin Polymorph A and one or more pharmaceutically acceptable carriers or excipients. In some embodiments, the disclosure provides a pharmaceutical formulation comprising high purity crystalline psilocybin Polymorph A′ and one or more pharmaceutically acceptable carriers or excipients.

Preferred pharmaceutical excipients for an oral formulation include: diluents, such as microcrystalline cellulose, starch, mannitol, calcium hydrogen phosphate anhydrous or co-mixtures of silicon dioxide, calcium carbonate, microcrystalline cellulose and talc; disintegrants, such as sodium starch glycolate or croscarmellose sodium; binders, such as povidone, co-povidone or hydroxyl propyl cellulose; lubricants, such as magnesium stearate or sodium stearyl fumarate; glidants, such as colloidal silicon dioxide; and film coats, such as Opadry II white or PVA based brown Opadry II.

In some embodiments, the pharmaceutical formulation is a parenteral dosage form. In some embodiments, the pharmaceutical formulation is an oral dosage form. In some embodiments, the pharmaceutical composition comprises a tablet. In some embodiments, the pharmaceutical composition comprises a capsule.

In some embodiments, the oral dosage form comprises a functional filler. The functional filler may be a silicified filler, such as, but not limited to silicified microcrystalline cellulose (SMCC). In some embodiments, the oral dosage form comprises high compactability grades of SMCC with a particle size range of from about 45 to 150 microns. A mixture of two functional fillers having different particle size ranges may be used with the weight percentages of the two favoring the larger sized particles.

In some embodiments, the silicified microcrystalline filler may comprise a first filler, having a particle size range of from about 45 to 80 microns in an amount of up to 30%, up to 20%, up to 15%, or less by weight of filler, and a second filler, having a particle size range of from about 90 to 150 microns, in an amount of up to 70%, up to 80%, up to 85%, or more, by weight of filler.

In some embodiments, the oral dosage form may comprise silicified microcrystalline cellulose with a particle size range of from about 45 to 80 microns (SMCC 50), such as Prosolv 50; silicified microcrystalline cellulose with a particle size range of from about 90 to 150 microns (SMCC 90), such as Prosolv 90; or mixtures thereof. In other embodiments, the oral dosage form may comprise SMCC 50 and SMCC 90. In other embodiments, the oral dosage form may comprise SMCC 50 and SMCC 90, wherein the ratio of SMCC 50 to SMCC 90 is 1:5 to 1:8 wt %. In still other embodiments the ratio of SMCC 50 to SMCC 90 is 1:5-1:7; 1:6-1:7; 1:6-1:8; or 1.7-1.8. In still other embodiments the ratio of SMCC 50 to SMCC 90 is 1:6; 1:6.1; 1:6.2; 1:6.3; 1:6.4; 1:6.5; 1:6.6; 1.6.7; 1:6.8; 1.6.9; or 1:7. The formulation may further comprise or consist essentially of a disintegrant, including without limitation sodium starch glycolate; a glidant, including without limitation colloidal silicon dioxide; and a lubricant, including without limitation sodium stearyl fumarate.

In some embodiments, the oral dosage form may comprise a disintegrant such as sodium starch glycolate, at less than 3% (by wt), less than 2%, or 1% or less.

In some embodiments, the oral dosage form comprises 5 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 5 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 5 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 10 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 10 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 10 mg of psilocybin and SMCC 50 and SMCC 90, wherein the ratio of SMCC 50 to SMCC 90 is 1:6.4 and sodium starch glycolate at about 0.5%.

In some embodiments, the oral dosage form comprises 25 mg of psilocybin and SMCC 50 and SMCC 90, wherein the ratio of SMCC 50 to SMCC 90 is 1:6.4 and sodium starch glycolate at about 1%. In some embodiments, the oral dosage form comprises 25 mg of psilocybin and SMCC 50 and SMCC 90, wherein the ratio of SMCC 50 to SMCC 90 is 1:6.4 and sodium starch glycolate at about 0.5% to 1.0%. In some embodiments, the oral dosage form comprises 25 mg of psilocybin and SMCC 50 and SMCC 90, wherein the ratio of SMCC 50 to SMCC 90 is 1:6.4 and sodium starch glycolate at about 0.5%.

In some embodiments, the tablet or capsule comprises one or more excipients. Non-limiting exemplary excipients include microcrystalline cellulose and starch, including without limitation silicified microcrystalline cellulose.

It should be noted that the formulations may comprise psilocybin in any form, not only the polymorphic forms disclosed herein.

As used herein, oral doses of psilocybin are classified follows: “very low doses” (about 0.045 mg/kg or less); “low doses” (between about 0.115 and about 0.125 mg/kg), “medium doses” (between about 0.115 to about 0.260 mg/kg), and “high doses” (about 0.315 mg/kg or more). See Studerus et al (2011) J Psychopharmacol 25(11) 1434-1452.

In some embodiments, the formulated dose of psilocybin comprises from about 0.01 mg/kg to about 1 mg/kg. In some embodiments, a human dose (for an adult weighing 60-80 kg) comprises between about 0.60 mg and about 80 mg.

In some embodiments, a formulated dose comprises between about 2 and about 50 mg of crystalline psilocybin. In some embodiments, a formulated dose comprises between 2 and 40 mg, between 2 and 10 mg, between 5 and 30 mg, between 5 and 15 mg, or between 20 and 30 mg of crystalline psilocybin. In some embodiments, a formulated dose comprises about 1 mg, about 5 mg, about 10 mg, or about 25 mg of crystalline psilocybin.

In some embodiments, a formulated dose comprises between about 2 and about 50 mg of crystalline psilocybin Polymorph A or Polymorph A′ or a mixture thereof. In some embodiments, a formulated dose comprises between 2 and 40 mg, between 2 and 10 mg, between 5 and 30 mg, between 5 and 15 mg, or between 20 and 30 mg of crystalline psilocybin Polymorph A or Polymorph A′ or a mixture thereof. In some embodiments, a formulated dose comprises about 1 mg, about 5 mg, about 10 mg, or about 25 mg of crystalline psilocybin Polymorph A or Polymorph A′ or a mixture thereof.

In some embodiments, a formulated dose comprises between about 2 and about 50 mg of crystalline psilocybin Polymorph A. In some embodiments, a formulated dose comprises between 2 and 40 mg, between 2 and 10 mg, between 5 and 30 mg, between 5 and 15 mg, or between 20 and 30 mg of crystalline psilocybin Polymorph A. In some embodiments, a formulated dose comprises about 1 mg, about 5 mg, about 10 mg, or about 25 mg of crystalline psilocybin Polymorph A.

In some embodiments, a formulated dose comprises between about 2 and about 50 mg of crystalline psilocybin Polymorph A′. In some embodiments, a formulated dose comprises between 2 and 40 mg, between 2 and 10 mg, between 5 and 30 mg, between 5 and 15 mg, or between 20 and 30 mg of crystalline psilocybin Polymorph A′. In some embodiments, a formulated dose comprises about 1 mg, about 5 mg, about 10 mg, or about 25 mg of crystalline psilocybin Polymorph A′.

In some embodiments, a formulated dose comprises between about 2 and about 50 mg of crystalline psilocybin Polymorph B. In some embodiments, a formulated dose comprises between 2 and 40 mg, between 2 and 10 mg, between 5 and 30 mg, between 5 and 15 mg, or between 20 and 30 mg of crystalline psilocybin Polymorph B. In some embodiments, a formulated dose comprises about 1 mg, about 5 mg, about 10 mg, or about 25 mg of crystalline psilocybin Polymorph B.

In some embodiments, a formulated dose comprises between about 2 and about 50 mg of crystalline psilocybin Hydrate A. In some embodiments, a formulated dose comprises between 2 and 40 mg, between 2 and 10 mg, between 5 and 30 mg, between 5 and 15 mg, or between 20 and 30 mg of crystalline psilocybin Hydrate A. In some embodiments, a formulated dose comprises about 1 mg, about 5 mg, about 10 mg, or about 25 mg of crystalline psilocybin Hydrate A.

### Psilocybin Dosing

In some embodiments, psilocybin is administered to the patient at a dose of between about 0.1 mg to about 100 mg, about 1 mg to about 50 mg, or about 5 mg to about 30 mg. In some embodiments, psilocybin is administered to the patient at a dose of about 1 mg, about 10 mg, or about 25 mg.

In some embodiments, an adult oral dose comprises about 1 mg to about 40 mg, about 2 to about 30 mg, or about 15 to about 30 mg of crystalline psilocybin, for example about 1 mg, about 5 mg, about 10 mg, or about 25 mg of crystalline psilocybin. In some embodiments, a “micro-dose” of psilocybin is administered to a patient.

A micro-dose may comprise, for example, about 0.05 mg to about 2.5 mg of crystalline psilocybin, such as about 1.0 mg. In the case of micro-dosing the regime may comprise a regular, continuous regime of, for example, daily administration, every other day administration, or weekly, administration. Such dosing may be absent of psychotherapy support.

Psilocybin may be administered 1, 2, 3, 4, 5, 6, 7, 8, 9, or 10 times to a patient. In some embodiments, psilocybin is administered at least once to the patient. In some embodiments, psilocybin is administered at least twice to the patient. In some embodiments, psilocybin is administered multiple times to a patient.

In some embodiments, psilocybin is administered multiple times to a patient (e.g., 2, 3, 4, 5, 6, 7, 8, 9, or 10 times) at therapeutically effective intervals. In some embodiments, a therapeutically effective interval may be about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In some embodiments, a therapeutically effective interval may be about 1 month, about 3 months, about 6 months, or about 12 months. In some embodiments, the same dose of psilocybin is administered to a patient during each administration. In some embodiments, a different dose of psilocybin is administered to a patient during each administration. In some embodiments, the dose of psilocybin administered to the patient is increased over time. In some embodiments, the dose of psilocybin administered to the patient is decreased over time.

### Antidepressants (e.g., SSRIs)

In some embodiments, the patient is administered an antidepressant (AD) prior to administration of psilocybin. In some embodiments, the AD is selected from an SSRI, serotonin norepinephrine reuptake inhibitors, tricyclic antidepressants, monoamine oxidase inhibitors, mirtazapine, bupropion, lamotrigine, and an atypical antipsychotics. In some embodiments, the antidepressant is an SSRI. In some embodiments, the SSRI is citalopram, escitalopram, paroxetine, sertraline, fluvoxamine, fluoxetine, dapoxetine, indalpine, zimelidine, alaproclate, centpropazine, cericlamine, femoxetine, ifoxetine, omiloxetine, panuramine, pirandamine, seproxetine, or combinations thereof. In some embodiments, the SSRI is citalopram, escitalopram, paroxetine, sertraline, fluvoxamine, fluoxetine, or combinations thereof. In some embodiments, the AD is a tricyclic AD. In some embodiments, the tricyclic AD is selected from the group consisting of amitriptyline, imipramine, and nortriptyline. In some embodiments, the AD is a serotonin norepinephrine reuptake inhibitor. In some embodiments, the serotonin norepinephrine reuptake inhibitors are selected from the group consisting of venlafaxine and duloxetine. In some embodiments, the AD is a monoamine oxidase inhibitor. In some embodiments, the monoamine oxidase inhibitor is selected from the group consisting of phenelzine and tranylcypromine. In some embodiments, the AD is an atypical antipsychotic. In some embodiments, the atypical antipsychotic is selected from the group consisting of mianserin, lurasidone, aripiprazole, risperidone, olanzapine, quetiapine, ziprasidone, clozapine, iloperidone, paliperidone, asenapine, and olanzapine/fluoxetine.

In some embodiments, the AD is selected from the following group: amitriptyline, amoxapine, clomipramine, desipramine, dosulepin, doxepin, imipramine, lofepramine, nortriptyline, protriptyline, tianeptine, trimipramine, isocarboxazid, phenelzine, tranylcypromine, moclobemide, selegiline, maprotiline, mianserin, mirtazapine, nefazadone, trazodone, vilazodone, vortioxetine, bupropion, agomelatine, flupentixol, ketamine, and mixtures thereof.

In the embodiments described herein, reference is made to the dose and methods of dose reduction of SSRIs. However, the present disclosure contemplates the disclosed doses and methods of dose reduction for any of the ADs described herein.

In some embodiments, prior to administration of psilocybin, the AD is administered chronically. In some embodiments, chronic administration of an AD is administration of an AD for at least 2 weeks, for example, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 3 months, at least 4 months, at least 5 months, at least 6 months, at least 7 months, at least 8 months, at least 9 months, at least 10 months, at least 11 months, at least 12 months, or more. In some embodiments, chronic administration of an AD is administration of an AD for at least 6 weeks, for example, at least 6 weeks, at least 7 weeks, at least 8 weeks, at least 9 weeks, at least 10 weeks, at least 11 weeks, at least 12 weeks, at least 3 months, at least 4 months, at least 5 months, at least 6 months, at least 7 months, at least 8 months, at least 9 months, at least 10 months, at least 11 months, at least 12 months, or more.

In some embodiments, prior to administration of psilocybin, a maintenance dose of between about 10 mg/day and about 250 mg/day of AD is administered, including all subranges and values thereof, for example, about 10 mg/day to about 20 mg/day, about 10 mg/day to about 30 mg/day, about 10 mg/day to about 50 mg/day, about 20 mg/day to about 100 mg/day, about 20 mg/day to about 40 mg/day, about 20 mg/day to about 80 mg/day, about 20 mg/day to about 60 mg/day, about 10 mg/day to about 60 mg/day, about 30 mg/day to about 60 mg/day, about 100 mg/day to about 200 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 105 mg/day, about 110 mg/day, about 115 mg/day, about 120 mg/day, about 125 mg/day, about 130 mg/day, about 135 mg/day, about 140 mg/day, about 145 mg/day, about 150 mg/day, about 155 mg/day, about 160 mg/day, about 165 mg/day, about 170 mg/day, about 175 mg/day, about 180 mg/day, about 185 mg/day, about 190 mg/day, about 195 mg/day, or about 200 mg/day of AD is administered.

In some embodiments, prior to administration of psilocybin, a maintenance dose of one or more ADs is administered chronically. In some embodiments, this chronically administered maintenance dose is any of the maintenance doses disclosed herein administered for any of the chronic administration periods disclosed herein, such as, but not limited to: about 4 weeks, about 5 weeks, about 6 weeks, about 7 weeks, about 8 weeks, about 9 weeks, about 10 weeks, about 11 weeks, about 12 weeks, or more.

In some embodiments, about 1-35 days before psilocybin treatment, a patient is administered a reduced dose of an AD. In some embodiments, administration of the reduced dose of AD occurs for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, or about 35 days prior to administration of psilocybin. In some embodiments, the reduced dose of an AD is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the maintenance dose of an AD.

In some embodiments, about 1-35 days before psilocybin treatment, administration of an AD to the patient in need thereof is ceased. In some embodiments, about 1-21 days before psilocybin treatment, administration of an AD to the patient in need thereof is ceased. In some embodiments, administration of an AD to the patient in need thereof is ceased, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, or about 35 days prior to administration of psilocybin. In some embodiments, administration of an AD to the patient in need thereof is ceased about 2 weeks before administration of psilocybin. In some embodiments, administration of an AD to the patient in need thereof is ceased at least about 2 weeks before administration of psilocybin.

In some embodiments, cessation of an AD is immediate. For example, a patient that ceases an AD immediately takes the maintenance dose of an AD on one day and on the subsequent day does not take any AD.

In some embodiments, AD cessation occurs during a titration period. In some embodiments, the titration period is the length of time between administration of a maintenance dose of an AD and cessation of the AD. In some embodiments, the titration period is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, or about 35 days, or more. In some embodiments, the titration period is about 1 day to about 3 months, for example, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 6 weeks, about 7 weeks, about 8 weeks, about 9 weeks, about 10 weeks, about 11 weeks, about 12 weeks, about 13 weeks, about 14 weeks, about 15 weeks, about 1 month, about 2 months, or about 3 months. In some embodiments, the titration period is about 4 weeks. In some embodiments, the titration period is at least about 4 weeks.

In some embodiments, prior to administration of psilocybin, the SSRI is administered chronically. In some embodiments, chronic administration of an SSRI is administration of an SSRI for at least 2 weeks, for example, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 3 months, at least 4 months, at least 5 months, at least 6 months, at least 7 months, at least 8 months, at least 9 months, at least 10 months, at least 11 months, at least 12 months, or more. In some embodiments, chronic administration of an SSRI is administration of an SSRI for at least 6 weeks, for example, at least 6 weeks, at least 7 weeks, at least 8 weeks, at least 9 weeks, at least 10 weeks, at least 11 weeks, at least 12 weeks, at least 3 months, at least 4 months, at least 5 months, at least 6 months, at least 7 months, at least 8 months, at least 9 months, at least 10 months, at least 11 months, at least 12 months, or more.

In some embodiments, prior to administration of psilocybin, a maintenance dose of between about 10 mg/day and about 250 mg/day of SSRI is administered, including all subranges and values thereof, for example, about 10 mg/day to about 20 mg/day, about 10 mg/day to about 30 mg/day, about 10 mg/day to about 50 mg/day, about 20 mg/day to about 100 mg/day, about 20 mg/day to about 40 mg/day, about 20 mg/day to about 80 mg/day, about 20 mg/day to about 60 mg/day, about 10 mg/day to about 60 mg/day, about 30 mg/day to about 60 mg/day, about 100 mg/day to about 200 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 105 mg/day, about 110 mg/day, about 115 mg/day, about 120 mg/day, about 125 mg/day, about 130 mg/day, about 135 mg/day, about 140 mg/day, about 145 mg/day, about 150 mg/day, about 155 mg/day, about 160 mg/day, about 165 mg/day, about 170 mg/day, about 175 mg/day, about 180 mg/day, about 185 mg/day, about 190 mg/day, about 195 mg/day, or about 200 mg/day of SSRI is administered.

In some embodiments, prior to administration of psilocybin, a maintenance dose of one or more SSRIs is administered chronically. In some embodiments, this chronically administered maintenance dose is any of the maintenance doses disclosed herein administered for any of the chronic administration periods disclosed herein, such as, but not limited to: about 4 weeks, about 5 weeks, about 6 weeks, about 7 weeks, about 8 weeks, about 9 weeks, about 10 weeks, about 11 weeks, about 12 weeks, or more.

In some embodiments, about 1-35 days before psilocybin treatment, a patient is administered a reduced dose of an SSRI. In some embodiments, administration of the reduced dose of SSRI occurs for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, or about 35 days prior to administration of psilocybin. In some embodiments, the reduced dose of an SSRI is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the maintenance dose of an SSRI.

In some embodiments, about 1-35 days, about 1-21 days, or about 1-14 days before psilocybin treatment, administration of an SSRI to the patient in need thereof is ceased. In some embodiments, about 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 days before psilocybin treatment, administration of an SSRI to the patient in need thereof is ceased. In some embodiments, about 1-14 days before psilocybin treatment, administration of an SSRI to the patient in need thereof is ceased. In some embodiments, about 1-12 days before psilocybin treatment, administration of an SSRI to the patient in need thereof is ceased. In some embodiments, administration of an SSRI to the patient in need thereof is ceased, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, or about 35 days prior to administration of psilocybin.

In some embodiments, cessation of an SSRI is immediate. For example, a patient that ceases an SSRI immediately takes the maintenance dose of an SSRI on one day and on the subsequent day does not take any SSRI.

In some embodiments, SSRI cessation occurs during a titration period. In some embodiments, the titration period is the length of time between administration of a maintenance dose of an SSRI and cessation of the SSRI. In some embodiments, the titration period is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, or about 35 days, or more. In some embodiments, the titration period is about 1 day to about 3 months, for example, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 6 weeks, about 7 weeks, about 8 weeks, about 9 weeks, about 10 weeks, about 11 weeks, about 12 weeks, about 13 weeks, about 14 weeks, about 15 weeks, about 1 month, about 2 months, or about 3 months. In some embodiments, the titration period is about 4 weeks. In some embodiments, the titration period is at least about 4 weeks.

### Fluoxetine

In some embodiments, the patient is administered a maintenance dose of fluoxetine (PROZAC®) prior to administration of psilocybin. In some embodiments, the maintenance dose of fluoxetine is between about 20 mg per day and about 60 mg per day, for example, about 20 mg/day, about 30 mg/day, about 40 mg/day, about 50 mg/day, about 60 mg/day. In some embodiments, the maintenance dose of fluoxetine is between about 20 mg per day and about 80 mg per day, for example, about 20 mg/day, about 30 mg/day, about 40 mg/day, about 50 mg/day, about 60 mg/day, about 70 mg/day, or about 80 mg/day. In some embodiments, between about 1 and about 35 days before administration of psilocybin, administration of fluoxetine to the patient in need is ceased. In some embodiments, administration of fluoxetine to the patient in need is ceased at least 2 weeks prior to administration of psilocybin. In some embodiments, cessation of fluoxetine is immediate. In some embodiments, cessation of fluoxetine occurs during a titration period. In some embodiments, the titration period is between about 1 week and about 3 months, for example, about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 1 month, 2 month, or 3 months.

In some embodiments, the dose of fluoxetine is reduced from the patient's maintenance dose by about 10 mg/day every three days, every four days, every week, every other week, every third week, every fourth week, or every month. In some embodiments, the dose of fluoxetine is reduced from the patient's maintenance dose by about 20 mg/day every week, every three days, every four days, every week, every other week, every third week, every fourth week, or every month. In some embodiments, the dose of fluoxetine is reduced from the patient's maintenance dose by about 30 mg/day every three days, every four days, every week, every other week, every third week, every fourth week, or every month. In some embodiments, the dose of fluoxetine is reduced from the patient's maintenance dose by about 40 mg/day every three days, every four days, every week, every other week, every third week, every fourth week, or every month.

In some embodiments, the dose of fluoxetine is reduced from the patient's maintenance dose by about 10% to about 75%, and all subranges there between, for example, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, about 10%, about 15%, about 20%, about 25%, about 30%, about 35%, about 40%, about 45%, about 50%, about 55%, about 60%, about 65%, about 70%, or about 75% every three days, every four days, every week, every other week, every third week, every fourth week, or every month. In some embodiments, the dose of fluoxetine is reduced from the patient's maintenance dose by about 10% every three days, every four days, every week, every other week, every third week, every fourth week, or every month. In some embodiments, the dose of fluoxetine is reduced from the patient's maintenance dose by about 25% every three days, every four days, every week, every other week, every third week, every fourth week, or every month. In some embodiments, the dose of fluoxetine is reduced from the patient's maintenance dose by about 50% every three days, every four days, every week, every other week, every third week, every fourth week, or every month.

### Citalopram

In some embodiments, the patient is administered a maintenance dose of citalopram (Celexa®) prior to administration of psilocybin. In some embodiments, the maintenance dose of citalopram is between about 10 mg/day and about 80 mg/day, for example, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, or about 80 mg/day. In some embodiments, the maintenance dose of citalopram is between about 20 mg/day and about 40 mg/day, for example, about 20 mg/day, about 25 mg/day, about 30 mg/day, about 35 mg/day, or about 40 mg/day.

In some embodiments, between about 1 and about 35 days before administration of psilocybin, administration of citalopram to the patient in need is ceased. In some embodiments, cessation of citalopram is immediate. In some embodiments, cessation of citalopram occurs during a titration period. In some embodiments, the titration period is between about 1 week and about 3 months, for example, about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 1 month, 2 month, or 3 months. In some embodiments, the dose of citalopram is reduced from the patient's maintenance dose by about 10 mg/day every three days, every four days, every week, every other week, every third week, every fourth week, or every month. In some embodiments, the dose of citalopram is reduced from the patient's maintenance dose by about 20 mg/day every three days, every four days, every week, every other week, every third week, every fourth week, or every month. In some embodiments, the dose of citalopram is reduced from the patient's maintenance dose by about 30 mg/day every three days, every four days, every week, every other week, every third week, every fourth week, or every month. In some embodiments, the dose of citalopram is reduced from the patient's maintenance dose by about 40 mg/day every three days, every four days, every week, every other week, every third week, every fourth week, or every month.

In some embodiments, the dose of citalopram is reduced from the patient's maintenance dose by about 10% to about 75%, and all subranges there between, for example, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, about 10%, about 15%, about 20%, about 25%, about 30%, about 35%, about 40%, about 45%, about 50%, about 55%, about 60%, about 65%, about 70%, or about 75% every three days, every four days, every week, every other week, every third week, every fourth week, or every month. In some embodiments, the dose of citalopram is reduced from the patient's maintenance dose by about 10% every three days, every four days, every week, every other week, every third week, every fourth week, or every month. In some embodiments, the dose of citalopram is reduced from the patient's maintenance dose by about 25% every three days, every four days, every week, every other week, every third week, every fourth week, or every month. In some embodiments, the dose of citalopram is reduced from the patient's maintenance dose by about 50% every three days, every four days, every week, every other week, every third week, every fourth week, or every month.

### Escitalopram

In some embodiments, the patient is administered a maintenance dose of escitalopram prior to administration of psilocybin. In some embodiments, the escitalopram is escitalopram oxalate. In some embodiments, the maintenance dose of escitalopram is between about 10 mg/day and about 20 mg/day, for example, about 10 mg/day, about 11 mg/day, about 12 mg/day, about 13 mg/day, about 14 mg/day, about 15 mg/day, about 16 mg/day, about 17 mg/day, about 18 mg/day, about 19 mg/day, or about 20 mg/day. In some embodiments, the maintenance dose of escitalopram is 10 mg/day. In some embodiments, the maintenance dose of escitalopram is 20 mg/day.

In some embodiments, between about 1 and about 35 days before administration of psilocybin, administration of escitalopram to the patient in need is ceased. In some embodiments, cessation of escitalopram is immediate. In some embodiments, cessation of escitalopram occurs during a titration period. In some embodiments, the titration period is between about 1 week and about 3 months, for example, about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 1 month, 2 month, or 3 months. In some embodiments, the dose of escitalopram is reduced from the patient's maintenance dose by about 1 mg/day every three days, every four days, every week, every other week, every third week, every fourth week, or every month. In some embodiments, the dose of escitalopram is reduced from the patient's maintenance dose by about 2 mg/day every three days, every four days, every week, every other week, every third week, every fourth week, or every month. In some embodiments, the dose of escitalopram is reduced from the patient's maintenance dose by about 3 mg/day every three days, every four days, every week, every other week, every third week, every fourth week, or every month. In some embodiments, the dose of escitalopram is reduced from the patient's maintenance dose by about 4 mg/day every three days, every four days, every week, every other week, every third week, every fourth week, or every month. In some embodiments, the dose of escitalopram is reduced from the patient's maintenance dose by about 5 mg/day every three days, every four days, every week, every other week, every third week, every fourth week, or every month. In some embodiments, the dose of escitalopram is reduced from the patient's maintenance dose by about 6 mg/day every three days, every four days, every week, every other week, every third week, every fourth week, or every month. In some embodiments, the dose of escitalopram is reduced from the patient's maintenance dose by about 7 mg/day every three days, every four days, every week, every other week, every third week, every fourth week, or every month. In some embodiments, the dose of escitalopram is reduced from the patient's maintenance dose by about 8 mg/day every three days, every four days, every week, every other week, every third week, every fourth week, or every month. In some embodiments, the dose of escitalopram is reduced from the patient's maintenance dose by about 9 mg/day every three days, every four days, every week, every other week, every third week, every fourth week, or every month. In some embodiments, the dose of escitalopram is reduced from the patient's maintenance dose by about 10 mg/day every three days, every four days, every week, every other week, every third week, every fourth week, or every month.

In some embodiments, the dose of escitalopram is reduced from the patient's maintenance dose by about 10% to about 75%, and all subranges there between, for example, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, about 10%, about 15%, about 20%, about 25%, about 30%, about 35%, about 40%, about 45%, about 50%, about 55%, about 60%, about 65%, about 70%, or about 75% every three days, every four days, every week, every other week, every third week, every fourth week, or every month. In some embodiments, the dose of escitalopram is reduced from the patient's maintenance dose by about 10% every three days, every four days, every week, every other week, every third week, every fourth week, or every month. In some embodiments, the dose of escitalopram is reduced from the patient's maintenance dose by about 20% every three days, every four days, every week, every other week, every third week, every fourth week, or every month. In some embodiments, the dose of escitalopram is reduced from the patient's maintenance dose by about 25% every three days, every four days, every week, every other week, every third week, every fourth week, or every month. In some embodiments, the dose of escitalopram is reduced from the patient's maintenance dose by about 50% every three days, every four days, every week, every other week, every third week, every fourth week, or every month.

### Paroxetine

In some embodiments, the patient is administered a maintenance dose of paroxetine (PAXIL®) prior to administration of psilocybin. In some embodiments, the maintenance dose of paroxetine is between about 20 mg/day and about 50 mg/day, for example, about 20 mg/day, about 25 mg/day, about 30 mg/day, about 35 mg/day, about 40 mg/day, about 45 mg/day, or about 50 mg/day. In some embodiments, the maintenance dose of paroxetine is about 20 mg/day. In some embodiments, the maintenance dose of paroxetine is about 40 mg/day.

In some embodiments, between about 1 and about 35 days before administration of psilocybin, administration of paroxetine to the patient in need is ceased. In some embodiments, cessation of paroxetine is immediate. In some embodiments, cessation of paroxetine occurs during a titration period. In some embodiments, the titration period is between about 1 week and about 3 months, for example, about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 1 month, 2 month, or 3 months. In some embodiments, the dose of paroxetine is reduced from the patient's maintenance dose by about 5 mg/day every three days, every four days, every week, every other week, every third week, every fourth week, or every month. In some embodiments, the dose of paroxetine is reduced from the patient's maintenance dose by about 10 mg/day every three days, every four days, every week, every other week, every third week, every fourth week, or every month. In some embodiments, the dose of paroxetine is reduced from the patient's maintenance dose by about 15 mg/day every three days, every four days, every week, every other week, every third week, every fourth week, or every month. In some embodiments, the dose of paroxetine is reduced from the patient's maintenance dose by about 20 mg/day every three days, every four days, every week, every other week, every third week, every fourth week, or every month. In some embodiments, the dose of paroxetine is reduced from the patient's maintenance dose by 10 mg/day every week until a patient reaches a dose of 20 mg/day, at which point, the patient continues on this dose for one week, before treatment is ceased.

In some embodiments, the dose of paroxetine is reduced from the patient's maintenance dose by about 10% to about 75%, and all subranges there between, for example, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, about 10%, about 15%, about 20%, about 25%, about 30%, about 35%, about 40%, about 45%, about 50%, about 55%, about 60%, about 65%, about 70%, or about 75% every three days, every four days, every week, every other week, every third week, every fourth week, or every month. In some embodiments, the dose of paroxetine is reduced from the patient's maintenance dose by about 10% every three days, every four days, every week, every other week, every third week, every fourth week, or every month. In some embodiments, the dose of paroxetine is reduced from the patient's maintenance dose by about 20% every three days, every four days, every week, every other week, every third week, every fourth week, or every month. In some embodiments, the dose of paroxetine is reduced from the patient's maintenance dose by about 25% every three days, every four days, every week, every other week, every third week, every fourth week, or every month. In some embodiments, the dose of paroxetine is reduced from the patient's maintenance dose by about 50% every three days, every four days, every week, every other week, every third week, every fourth week, or every month.

### Sertraline

In some embodiments, the patient is administered a maintenance dose of sertraline (ZOLOFT®) prior to administration of psilocybin. In some embodiments, sertraline is sertraline hydrochloride. In some embodiments, the maintenance dose of sertraline is between about 50 mg/day and about 200 mg/day, for example, about 50 mg/day, about 62.5 mg/day, about 75 mg/day, about 87.5 mg/day, about 100 mg/day, about 112.5 mg/day, about 125 mg/day, about 137.5 mg/day, about 150 mg/day, about 162.5 mg/day, about 175 mg/day, about 187.5 mg/day, or about 200 mg/day. In some embodiments, the maintenance dose of sertraline is between about 25 mg/day and about 200 mg/day, for example, about 25 mg/day, about 37.5 mg/day, about 50 mg/day, about 62.5 mg/day, about 75 mg/day, about 87.5 mg/day, about 100 mg/day, about 112.5 mg/day, about 125 mg/day, about 137.5 mg/day, about 150 mg/day, about 162.5 mg/day, about 175 mg/day, about 187.5 mg/day, or about 200 mg/day.

In some embodiments, between about 1 and about 35 days before administration of psilocybin, administration of sertraline to the patient in need is ceased. In some embodiments, cessation of sertraline is immediate. In some embodiments, cessation of sertraline occurs during a titration period. In some embodiments, the titration period is between about 1 week and about 3 months, for example, about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 1 month, 2 month, or 3 months. In some embodiments, the dose of sertraline is reduced from the patient's maintenance dose by about 12.5 mg/day every three days, every four days, every week, every other week, every third week, every fourth week, or every month. In some embodiments, the dose of sertraline is reduced from the patient's maintenance dose by about 25 mg/day every three days, every four days, every week, every other week, every third week, every fourth week, or every month. In some embodiments, the dose of sertraline is reduced from the patient's maintenance dose by about 50 mg/day every three days, every four days, every week, every other week, every third week, every fourth week, or every month. In some embodiments, the dose of sertraline is reduced from the patient's maintenance dose by about 75 mg/day every three days, every four days, every week, every other week, every third week, every fourth week, or every month. In some embodiments, the dose of sertraline is reduced from the patient's maintenance dose by about 100 mg/day every three days, every four days, every week, every other week, every third week, every fourth week, or every month. In some embodiments, the dose of sertraline is reduced from the patient's maintenance dose by about 125 mg/day every three days, every four days, every week, every other week, every third week, every fourth week, or every month. In some embodiments, the dose of sertraline is reduced from the patient's maintenance dose by about 150 mg/day every three days, every four days, every week, every other week, every third week, every fourth week, or every month. In some embodiments, the dose of sertraline is reduced from the patient's maintenance dose by about 175 mg/day every three days, every four days, every week, every other week, every third week, every fourth week, or every month. In some embodiments, the dose of sertraline is reduced from the patient's maintenance dose by about 200 mg/day every three days, every four days, every week, every other week, every third week, every fourth week, or every month.

In some embodiments, the dose of sertraline is reduced from the patient's maintenance dose by about 10% to about 75%, and all subranges there between, for example, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, about 10%, about 15%, about 20%, about 25%, about 30%, about 35%, about 40%, about 45%, about 50%, about 55%, about 60%, about 65%, about 70%, or about 75% every three days, every four days, every week, every other week, every third week, every fourth week, or every month. In some embodiments, the dose of sertraline is reduced from the patient's maintenance dose by about 10% every three days, every four days, every week, every other week, every third week, every fourth week, or every month. In some embodiments, the dose of sertraline is reduced from the patient's maintenance dose by about 20% every three days, every four days, every week, every other week, every third week, every fourth week, or every month. In some embodiments, the dose of sertraline is reduced from the patient's maintenance dose by about 25% every three days, every four days, every week, every other week, every third week, every fourth week, or every month. In some embodiments, the dose of sertraline is reduced from the patient's maintenance dose by about 50% every three days, every four days, every week, every other week, every third week, every fourth week, or every month.

### Fluvoxamine

In some embodiments, the patient is administered a maintenance dose of fluvoxamine (Luvox®) prior to administration of psilocybin. In some embodiments, fluvoxamine is fluvoxamine maleate. In some embodiments, the maintenance dose of fluvoxamine is between about 25 mg/day and about 300 mg/day, for example, about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, or about 300 mg/day. In some embodiments, the maintenance dose of fluvoxamine is between about 50 mg/day and about 300 mg/day, for example, about 50 mg, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, or about 300 mg/day.

In some embodiments, between about 1 and about 35 days before administration of psilocybin, administration of fluvoxamine to the patient in need is ceased. In some embodiments, cessation of fluvoxamine is immediate. In some embodiments, cessation of fluvoxamine occurs during a titration period. In some embodiments, the titration period is between about 1 week and about 3 months, for example, about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 1 month, 2 month, or 3 months. In some embodiments, the dose of fluvoxamine is reduced from the patient's maintenance dose by about 12.5 mg/day every three days, every four days, every week, every other week, every third week, every fourth week, or every month. In some embodiments, the dose of fluvoxamine is reduced from the patient's maintenance dose by about 25 mg/day every three days, every four days, every week, every other week, every third week, every fourth week, or every month. In some embodiments, the dose of fluvoxamine is reduced from the patient's maintenance dose by about 50 mg/day every three days, every four days, every week, every other week, every third week, every fourth week, or every month. In some embodiments, the dose of fluvoxamine is reduced from the patient's maintenance dose by about 75 mg/day every three days, every four days, every week, every other week, every third week, every fourth week, or every month. In some embodiments, the dose of fluvoxamine is reduced from the patient's maintenance dose by about 100 mg/day every three days, every four days, every week, every other week, every third week, every fourth week, or every month. In some embodiments, the dose of fluvoxamine is reduced from the patient's maintenance dose by about 125 mg/day every three days, every four days, every week, every other week, every third week, every fourth week, or every month. In some embodiments, the dose of fluvoxamine is reduced from the patient's maintenance dose by about 150 mg/day every three days, every four days, every week, every other week, every third week, every fourth week, or every month. In some embodiments, the dose of fluvoxamine is reduced from the patient's maintenance dose by about 175 mg/day every three days, every four days, every week, every other week, every third week, every fourth week, or every month. In some embodiments, the dose of fluvoxamine is reduced from the patient's maintenance dose by about 200 mg/day every three days, every four days, every week, every other week, every third week, every fourth week, or every month. In some embodiments, the dose of fluvoxamine is reduced from the patient's maintenance dose by about 225 mg/day every three days, every four days, every week, every other week, every third week, every fourth week, or every month. In some embodiments, the dose of fluvoxamine is reduced from the patient's maintenance dose by about 250 mg/day every three days, every four days, every week, every other week, every third week, every fourth week, or every month.

In some embodiments, the dose of fluvoxamine is reduced from the patient's maintenance dose by about 10% to about 75%, and all subranges there between, for example, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, about 10%, about 15%, about 20%, about 25%, about 30%, about 35%, about 40%, about 45%, about 50%, about 55%, about 60%, about 65%, about 70%, or about 75% every three days, every four days, every week, every other week, every third week, every fourth week, or every month. In some embodiments, the dose of fluvoxamine is reduced from the patient's maintenance dose by about 10% every three days, every four days, every week, every other week, every third week, every fourth week, or every month. In some embodiments, the dose of fluvoxamine is reduced from the patient's maintenance dose by about 20% every three days, every four days, every week, every other week, every third week, every fourth week, or every month. In some embodiments, the dose of fluvoxamine is reduced from the patient's maintenance dose by about 25% every three days, every four days, every week, every other week, every third week, every fourth week, or every month. In some embodiments, the dose of fluvoxamine is reduced from the patient's maintenance dose by about 50% every three days, every four days, every week, every other week, every third week, every fourth week, or every month.

### Desvenlafaxine

In some embodiments, the patient is administered a maintenance dose of desvenlafaxine (PRISTIQ®) prior to administration of psilocybin. In some embodiments, the maintenance dose of desvenlafaxine is between about 20 mg/day and about 60 mg/day, for example, about 20 mg/day, about 25 mg/day, about 30 mg/day, about 35 mg/day, about 40 mg/day, about 45 mg/day, or about 50 mg/day. In some embodiments, the maintenance dose of desvenlafaxine is about 20 mg/day. In some embodiments, the maintenance dose of desvenlafaxine is about 40 mg/day.

In some embodiments, between about 1 and about 35 days before administration of psilocybin, administration of desvenlafaxine to the patient in need is ceased. In some embodiments, cessation of desvenlafaxine is immediate. In some embodiments, cessation of desvenlafaxine occurs during a titration period. In some embodiments, the titration period is between about 1 week and about 3 months, for example, about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 1 month, 2 month, or 3 months. In some embodiments, the dose of desvenlafaxine is reduced from the patient's maintenance dose by about 5 mg/day every three days, every four days, every week, every other week, every third week, every fourth week, or every month. In some embodiments, the dose of desvenlafaxine is reduced from the patient's maintenance dose by about 10 mg/day every three days, every four days, every week, every other week, every third week, every fourth week, or every month. In some embodiments, the dose of desvenlafaxine is reduced from the patient's maintenance dose by about 15 mg/day every three days, every four days, every week, every other week, every third week, every fourth week, or every month. In some embodiments, the dose of desvenlafaxine is reduced from the patient's maintenance dose by about 20 mg/day every three days, every four days, every week, every other week, every third week, every fourth week, or every month. In some embodiments, the dose of desvenlafaxine is reduced from the patient's maintenance dose by 10 mg/day every week until a patient reaches a dose of 20 mg/day, at which point, the patient continues on this dose for one week, before treatment is ceased.

In some embodiments, the dose of desvenlafaxine is reduced from the patient's maintenance dose by about 10% to about 75%, and all subranges there between, for example, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, about 10%, about 15%, about 20%, about 25%, about 30%, about 35%, about 40%, about 45%, about 50%, about 55%, about 60%, about 65%, about 70%, or about 75% every three days, every four days, every week, every other week, every third week, every fourth week, or every month. In some embodiments, the dose of desvenlafaxine is reduced from the patient's maintenance dose by about 10% every three days, every four days, every week, every other week, every third week, every fourth week, or every month. In some embodiments, the dose of desvenlafaxine is reduced from the patient's maintenance dose by about 20% every three days, every four days, every week, every other week, every third week, every fourth week, or every month. In some embodiments, the dose of desvenlafaxine is reduced from the patient's maintenance dose by about 25% every three days, every four days, every week, every other week, every third week, every fourth week, or every month. In some embodiments, the dose of desvenlafaxine is reduced from the patient's maintenance dose by about 50% every three days, every four days, every week, every other week, every third week, every fourth week, or every month.

### Duloxetine

In some embodiments, the patient is administered a maintenance dose of duloxetine (CYMBALTA®) prior to administration of psilocybin. In some embodiments, the maintenance dose of duloxetine is between about 20 mg/day and about 60 mg/day, for example, about 20 mg/day, about 25 mg/day, about 30 mg/day, about 35 mg/day, about 40 mg/day, about 45 mg/day, about 50 mg/day, about 55 mg/day, or about 60 mg/day. In some embodiments, the maintenance dose of duloxetine is about 20 mg/day. In some embodiments, the maintenance dose of duloxetine is about 40 mg/day.

In some embodiments, between about 1 and about 35 days before administration of psilocybin, administration of duloxetine to the patient in need is ceased. In some embodiments, cessation of duloxetine is immediate. In some embodiments, cessation of duloxetine occurs during a titration period. In some embodiments, the titration period is between about 1 week and about 3 months, for example, about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 1 month, 2 month, or 3 months. In some embodiments, the dose of duloxetine is reduced from the patient's maintenance dose by about 5 mg/day every three days, every four days, every week, every other week, every third week, every fourth week, or every month. In some embodiments, the dose of duloxetine is reduced from the patient's maintenance dose by about 10 mg/day every three days, every four days, every week, every other week, every third week, every fourth week, or every month. In some embodiments, the dose of duloxetine is reduced from the patient's maintenance dose by about 15 mg/day every three days, every four days, every week, every other week, every third week, every fourth week, or every month. In some embodiments, the dose of duloxetine is reduced from the patient's maintenance dose by about 20 mg/day every three days, every four days, every week, every other week, every third week, every fourth week, or every month. In some embodiments, the dose of duloxetine is reduced from the patient's maintenance dose by 10 mg/day every week until a patient reaches a dose of 20 mg/day, at which point, the patient continues on this dose for one week, before treatment is ceased.

In some embodiments, the dose of duloxetine is reduced from the patient's maintenance dose by about 10% to about 75%, and all subranges there between, for example, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, about 10%, about 15%, about 20%, about 25%, about 30%, about 35%, about 40%, about 45%, about 50%, about 55%, about 60%, about 65%, about 70%, or about 75% every three days, every four days, every week, every other week, every third week, every fourth week, or every month. In some embodiments, the dose of duloxetine is reduced from the patient's maintenance dose by about 10% every three days, every four days, every week, every other week, every third week, every fourth week, or every month. In some embodiments, the dose of duloxetine is reduced from the patient's maintenance dose by about 20% every three days, every four days, every week, every other week, every third week, every fourth week, or every month. In some embodiments, the dose of duloxetine is reduced from the patient's maintenance dose by about 25% every three days, every four days, every week, every other week, every third week, every fourth week, or every month. In some embodiments, the dose of duloxetine is reduced from the patient's maintenance dose by about 50% every three days, every four days, every week, every other week, every third week, every fourth week, or every month.

### Levomilnacipran

In some embodiments, the patient is administered a maintenance dose of levomilnacipran (FETZIMA®) prior to administration of psilocybin. In some embodiments, the maintenance dose of levomilnacipran is between about 50 mg/day and about 120 mg/day, for example, about 50 mg/day, about 62.5 mg/day, about 75 mg/day, about 87.5 mg/day, about 100 mg/day, about 112.5 mg/day, or about 120 mg/day. In some embodiments, the maintenance dose of levomilnacipran is between about 25 mg/day and about 120 mg/day, for example, about 25 mg/day, about 37.5 mg/day, about 50 mg/day, about 62.5 mg/day, about 75 mg/day, about 87.5 mg/day, about 100 mg/day, about 112.5 mg/day, or about 120 mg/day.

In some embodiments, between about 1 and about 35 days before administration of psilocybin, administration of levomilnacipran to the patient in need is ceased. In some embodiments, cessation of levomilnacipran is immediate. In some embodiments, cessation of levomilnacipran occurs during a titration period. In some embodiments, the titration period is between about 1 week and about 3 months, for example, about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 1 month, 2 month, or 3 months. In some embodiments, the dose of levomilnacipran is reduced from the patient's maintenance dose by about 12.5 mg/day every three days, every four days, every week, every other week, every third week, every fourth week, or every month. In some embodiments, the dose of levomilnacipran is reduced from the patient's maintenance dose by about 25 mg/day every three days, every four days, every week, every other week, every third week, every fourth week, or every month. In some embodiments, the dose of levomilnacipran is reduced from the patient's maintenance dose by about 50 mg/day every three days, every four days, every week, every other week, every third week, every fourth week, or every month. In some embodiments, the dose of levomilnacipran is reduced from the patient's maintenance dose by about 75 mg/day every three days, every four days, every week, every other week, every third week, every fourth week, or every month. In some embodiments, the dose of levomilnacipran is reduced from the patient's maintenance dose by about 60 mg/day every three days, every four days, every week, every other week, every third week, every fourth week, or every month. In some embodiments, the dose of levomilnacipran is reduced from the patient's maintenance dose by about 75 mg/day every three days, every four days, every week, every other week, every third week, every fourth week, or every month. In some embodiments, the dose of levomilnacipran is reduced from the patient's maintenance dose by about 90 mg/day every three days, every four days, every week, every other week, every third week, every fourth week, or every month. In some embodiments, the dose of levomilnacipran is reduced from the patient's maintenance dose by about 105 mg/day every three days, every four days, every week, every other week, every third week, every fourth week, or every month. In some embodiments, the dose of levomilnacipran is reduced from the patient's maintenance dose by about 120 mg/day every three days, every four days, every week, every other week, every third week, every fourth week, or every month.

In some embodiments, the dose of levomilnacipran is reduced from the patient's maintenance dose by about 10% to about 75%, and all subranges there between, for example, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, about 10%, about 15%, about 20%, about 25%, about 30%, about 35%, about 40%, about 45%, about 50%, about 55%, about 60%, about 65%, about 70%, or about 75% every three days, every four days, every week, every other week, every third week, every fourth week, or every month. In some embodiments, the dose of levomilnacipran is reduced from the patient's maintenance dose by about 10% every three days, every four days, every week, every other week, every third week, every fourth week, or every month. In some embodiments, the dose of levomilnacipran is reduced from the patient's maintenance dose by about 20% every three days, every four days, every week, every other week, every third week, every fourth week, or every month. In some embodiments, the dose of levomilnacipran is reduced from the patient's maintenance dose by about 25% every three days, every four days, every week, every other week, every third week, every fourth week, or every month. In some embodiments, the dose of levomilnacipran is reduced from the patient's maintenance dose by about 50% every three days, every four days, every week, every other week, every third week, every fourth week, or every month.

### Venflafaxine

In some embodiments, the patient is administered a maintenance dose of venflafaxine (EFFEXOR®) prior to administration of psilocybin. In some embodiments, the maintenance dose of venflafaxine is between about 50 mg/day and about 375 mg/day, for example, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day or about 375 mg/day. In some embodiments, the maintenance dose of venflafaxine is between about 25 mg/day and about 375 mg/day, for example, about 25 mg/day, about 50 mg/day, about 75 mg/day, 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day or about 375 mg/day.

In some embodiments, between about 1 and about 35 days before administration of psilocybin, administration of venflafaxine to the patient in need is ceased. In some embodiments, cessation of venflafaxine is immediate. In some embodiments, cessation of venflafaxine occurs during a titration period. In some embodiments, the titration period is between about 1 week and about 3 months, for example, about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 1 month, 2 month, or 3 months. In some embodiments, the dose of venflafaxine is reduced from the patient's maintenance dose by about 25 mg/day every three days, every four days, every week, every other week, every third week, every fourth week, or every month. In some embodiments, the dose of venflafaxine is reduced from the patient's maintenance dose by about 75 mg/day every three days, every four days, every week, every other week, every third week, every fourth week, or every month. In some embodiments, the dose of venflafaxine is reduced from the patient's maintenance dose by about 150 mg/day every three days, every four days, every week, every other week, every third week, every fourth week, or every month. In some embodiments, the dose of venflafaxine is reduced from the patient's maintenance dose by about 225 mg/day every three days, every four days, every week, every other week, every third week, every fourth week, or every month. In some embodiments, the dose of venflafaxine is reduced from the patient's maintenance dose by about 185 mg/day every three days, every four days, every week, every other week, every third week, every fourth week, or every month. In some embodiments, the dose of venflafaxine is reduced from the patient's maintenance dose by about 225 mg/day every three days, every four days, every week, every other week, every third week, every fourth week, or every month. In some embodiments, the dose of venflafaxine is reduced from the patient's maintenance dose by about 275 mg/day every three days, every four days, every week, every other week, every third week, every fourth week, or every month. In some embodiments, the dose of venflafaxine is reduced from the patient's maintenance dose by about 325 mg/day every three days, every four days, every week, every other week, every third week, every fourth week, or every month. In some embodiments, the dose of venflafaxine is reduced from the patient's maintenance dose by about 375 mg/day every three days, every four days, every week, every other week, every third week, every fourth week, or every month.

In some embodiments, the dose of venflafaxine is reduced from the patient's maintenance dose by about 10% to about 75%, and all subranges there between, for example, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 20% to about 75%, about 30% to about 40%, about 30% to about 50%, about 30% to about 60%, about 30% to about 70%, about 30% to about 75%, about 40% to about 50%, about 40% to about 60%, about 40% to about 70%, about 40% to about 75%, about 50% to about 60%, about 50% to about 70%, about 50% to about 75%, about 60% to about 70%, about 60% to about 75%, about 10%, about 15%, about 20%, about 25%, about 30%, about 35%, about 40%, about 45%, about 50%, about 55%, about 60%, about 65%, about 70%, or about 75% every three days, every four days, every week, every other week, every third week, every fourth week, or every month. In some embodiments, the dose of venflafaxine is reduced from the patient's maintenance dose by about 10% every three days, every four days, every week, every other week, every third week, every fourth week, or every month. In some embodiments, the dose of venflafaxine is reduced from the patient's maintenance dose by about 20% every three days, every four days, every week, every other week, every third week, every fourth week, or every month. In some embodiments, the dose of venflafaxine is reduced from the patient's maintenance dose by about 25% every three days, every four days, every week, every other week, every third week, every fourth week, or every month. In some embodiments, the dose of venflafaxine is reduced from the patient's maintenance dose by about 50% every three days, every four days, every week, every other week, every third week, every fourth week, or every month.

### Benzodiazepines

In some embodiments, a benzodiazepine is administered prior to administration of psilocybin. In some embodiments, the benzodiazepine is selected from the group consisting of adinazolam, alprazolam, bentazepam, bretazenil, bromazepam, bromazolam, brotizolam, camazepam, chlordiazepoxide, cinazepam, cinolazepam, clobazam, clonazepam, clonazolam, clorazepate, clotiazepam, cloxazolam, delorazepam, deschloroetizolam, diazepam, diclazepam, estazolam, ethyl carfluzepate, ethyl loflazepate, etizolam, flualprazolam, flubromazepam, flubromazolam, fluclotizolam, flunitrazepam, flunitrazolam, flurazepam, flutazolam, flutoprazepam, halazepam, ketazolam, loprazolam, lorazepam, lormetazepam, meclonazepam, medazepam, metizolam, mexazolam, midazolam, nifoxipam, nimetazepam, nitemazepam, nitrazepam, nitrazolam, nordiazepam, norflurazepam, oxazepam, phenazepam, pinazepam, prazepam, premazepam, pyrazolam, quazepam, rilmazafone, temazepam, tetrazepam, triazolam, or combinations thereof. In some embodiments, the benzodiazepine is selected from the group consisting of alprazolam, chlordiazepoxide, clonazepam, diazepam, lorazepam, oxazepam, or combinations thereof.

In some embodiments, a benzodiazepine is administered about 1 day to about 35 days before administration of psilocybin, including all subranges therebetween, for example, about 7 days to about 21 days, about 14 days to about 21 days, about 7 days to about 14 days, about 1 day to about 7 days, about 1 day to about 14 days, about 1 day to about 35 days, about 1 to about 21 days, about 21 days to about 35 days, about 28 days to about 35 days, about 21 days to about 28 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, or about 35 days before administration of psilocybin.

In some embodiments, a starting dose of between about 0.1 mg and about 50 mg of benzodiazepine is administered, including all subranges and values thereof, for example, about 0.25 mg to about 1 mg, about 5 mg to about 25 mg, about 0.5 mg to about 1 mg, about 10 mg to about 30 mg, about 0.1 mg, about 0.25 mg, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg daily, twice daily, three times daily, four times daily, five times daily, six times daily, seven times daily, eight times daily, nine times daily, or ten times daily.

In some embodiments, a maintenance dose of between about 0.1 mg and about 50 mg of benzodiazepine is administered, including all subranges and values thereof, for example, about 0.25 mg to about 1 mg, about 5 mg to about 25 mg, about 0.5 mg to about 1 mg, about 10 mg to about 30 mg, about 0.1 mg, about 0.25 mg, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg daily, twice daily, three times daily, four times daily, five times daily, six times daily, seven times daily, eight times daily, nine times daily, or ten times daily.

In some embodiments, the starting dose of benzodiazepine is equivalent to the maintenance dose of benzodiazepine. In some embodiments, a patient is immediately administered a maintenance dose without a titration period from a starting dose to the maintenance dose.

In some embodiments, a patient is administered a starting dose of benzodiazepine which is subsequently increased to a maintenance dose during a titration period. In some embodiments, during the titration period, a patient's starting dose is increased by about

In some embodiments, the starting or maintenance dose of alprazolam, chlordiazepoxide, clonazepam, diazepam, lorazepam, oxazepam, clorazepate, estazolam, flurazepam, midazolam, temazepam, triazolam, quazepam is a dose approved by the Food and Drug Administration as detailed in Table 7.

TABLE 7
FDA Approved Doses of Benzodiazepines

| Benzodiazepine | Approved Dose | Route |
|---|---|---|
| Alprazolam | 0.25 mg to 1 mg three times | oral |
| | a day (maximum 4 mg/day) | |
| chlordiazepoxide | 5 mg to 25 mg three times or | oral |
| | four times a day (maximum | |
| | 100 mg/day) | |
| Clonazepam | 0.5 mg to 1 mg three times | oral |
| | a day (maximum 20 mg/day) | |
| Diazepam | 5 mg to 25 mg three times a | Oral |
| | day or four times a day | |
| Lorazepam | 0.5 to 1 mg three times a | oral |
| | day or four times a day | |
| Oxazepam | 10 mg to 30 mg three times | Oral |
| | a day or four times a day | |
| | (maximum 120 mg/day) | |
| Clorazepate | Maximum 90 mg a day | Oral |
| Estazolam | 2 mg a day | Oral |
| Flurazepam | 30 mg a day | Oral |
| Midazolam | Maximum 10 mg a day | Oral |
| Temazepam | 40 mg a day | Oral |
| Triazolam | 0.5 mg a day | Oral |
| Quazepam | 15 mg a day | Oral |

In some embodiments, the starting or maintenance dose of alprazolam, chlordiazepoxide, clonazepam, diazepam, lorazepam, oxazepam, clorazepate, estazolam, flurazepam, midazolam, temazepam, triazolam, quazepam is based on the maintenance dose of an alternative benzodiazepine administered. The starting or maintenance dose may be calculated based on the alternative benzodiazepine's relative potency according to Table 8.

TABLE 8
Oral Dose Equivalences of Benzodiazepines

| Benzodiazepine | Relative Potency (mg) |
|---|---|
| Alprazolam | 0.5 |
| Chlordiazepoxide | 10 |
| Clonazepam | 0.25-0.5 |
| Diazepam | 5 |
| Lorazepam | 1 |
| oxazepam | 15-30 |
| Clorazepate | 7 |
| Estazolam | 0.5-1 |
| Flurazepam | 10 |
| Midazolam | 3.3 |
| Temazepam | 10 |
| Triazolam | 0.25 |
| Quazepam | 13 |

Administration of Benzodiazepine after Cessation of SSRI

In some embodiments, a starting dose of a benzodiazepine is administered after cessation of administration of an SSRI. In some embodiments, a starting dose of a benzodiazepine is administered after cessation of administration of an SSRI but prior to administration of psilocybin. In some embodiments, a starting dose of a benzodiazepine is administered about 1 day to about 21 days before administration of psilocybin, including all subranges therebetween, for example, about 7 days to about 21 days, about 14 days to about 21 days, about 7 days to about 14 days, about 1 day to about 7 days, about 1 day to about 14 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, or about 21 days before administration of psilocybin.

In some embodiments, the starting dose of a benzodiazepine is the maintenance dose of a benzodiazepine. In some embodiments, the dose of a benzodiazepine is titrated from the starting dose to a maintenance dose.

In some embodiments, the benzodiazepine is alprazolam. In some embodiments, the starting dose of alprazolam is between about 0.25 mg and about 1 mg of alprazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of alprazolam is between about 0.25 mg and about 1 mg of alprazolam, for example about 0.25 mg, about 0.5 mg, about 0.75 mg, or about 1 mg, and the starting dose of alprazolam is administered to a patient three times per day. In some embodiments, the starting dose of alprazolam is the maintenance dose of alprazolam. In some embodiments, the starting dose of alprazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of alprazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of alprazolam is increased at three to four day intervals by no more than 1 mg/day.

In some embodiments, the benzodiazepine is chlordiazepoxide. In some embodiments, the starting dose of chlordiazepoxide is between about 5 mg and about 25 mg, for example, about 5 mg, about 7.5 mg, about 10 mg, about 12.5 mg, about 15 mg, about 17.5 mg, about 20 mg, about 22.5 mg, or about 25 mg, and chlordiazepoxide is administered to a patient once a day, twice a day, three times per day, four times per day, five times per day, six times per day, seven times per day, eight times per day, nine times per day, or ten times per day. In some embodiments, the starting dose of chlordiazepoxide is 5 mg three times daily. In some embodiments, the starting dose of chlordiazepoxide is 10 mg three times daily. In some embodiments, the starting dose of chlordiazepoxide is 5 mg four times daily. In some embodiments, the starting dose of chlordiazepoxide is 10 mg four times daily. In some embodiments, the starting dose of chlordiazepoxide is 5 mg two times daily. In some embodiments, the starting dose of chlordiazepoxide is 5 mg four times daily. In some embodiments, the starting dose of chlordiazepoxide is 20 mg three times daily. In some embodiments, the starting dose of chlordiazepoxide is 20 mg four times daily. In some embodiments, the starting dose of chlordiazepoxide is 25 mg three times daily. In some embodiments, the starting dose of chlordiazepoxide is 25 mg four times daily. In some embodiments, the starting dose of chlordiazepoxide is the maintenance dose of chlordiazepoxide. In some embodiments, the starting dose of chlordiazepoxide is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg every day, every three to four days, every week, every other week, every third week, every fourth week, or every month until a maintenance dose is reached. In some embodiments, the starting dose of chlordiazepoxide is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is clonazepam. In some embodiments, a starting dose of between about 0.125 and about 1 mg, for example, about 0.125 mg, about 0.25 mg, about 0.5 mg, about 0.75 mg, or about 1 mg, of clonazepam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of clonazepam is administered to a patient three times per day. In some embodiments, the starting dose of clonazepam is the maintenance dose of clonazepam. In some embodiments, the starting dose of clonazepam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of clonazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of clonazepam is increased in increments of 0.125 to 0.25 mg twice a day every three days. In some embodiments, the maintenance dose of clonazepam is 1 mg per day.

In some embodiments, the benzodiazepine is diazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg diazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of diazepam two to four times daily. In some embodiments, a patient is administered a starting dose of about 10 mg of diazepam three to four times daily. In some embodiments, a patient is administered a starting dose of about 5 mg three times, or four times daily as needed. In some embodiments, the starting dose of diazepam is the maintenance dose of diazepam. In some embodiments, the starting dose of diazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of diazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

n some embodiments, the benzodiazepine is lorazepam. In some embodiments, a patient is administered a starting dose of between about 0.5 mg and 6 mg of lorazepam, for example, about 0.5 mg, about 0.75 mg, about 1 mg, about 1.5 mg, about 2 mg, about 2.5 mg, about 3 mg, about 3.5 mg, about 4 mg, about 4.5 mg, about 5 mg, about 5.5 mg, or about 6 mg of lorazepam once a day, twice a day, three times per day, four times per day, five times per day, six times per day, seven times per day, eight times per day, nine times per day, or ten times per day. In some embodiments, a patient is administered a starting dose of lorazepam of between about 2 mg/day to 4 mg/day. In some embodiments, patients are administered a starting dose of between about 2 mg/day to 3 mg/day given two times a day or three times a day. In some embodiments, the benzodiazepine is lorazepam. In some embodiments, the starting dose of lorazepam is the maintenance dose of lorazepam. In some embodiments, the starting dose of lorazepam is increased by between about 0.5 mg and 6 mg of lorazepam, for example, about 0.5 mg, about 0.75 mg, about 1 mg, about 1.5 mg, about 2 mg, about 2.5 mg, about 3 mg, about 3.5 mg, about 4 mg, about 4.5 mg, about 5 mg, about 5.5 mg, or about 6 mg, every day, every three to four days, every week, every other week, every third week, every fourth week, or every month until a maintenance dose is reached. In some embodiments, the starting dose of lorazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is clorazepate. In some embodiments, the starting dose of clorazepate is between about 5 mg and about 25 mg, for example, about 5 mg, about 7.5 mg, about 10 mg, about 12.5 mg, about 15 mg, about 17.5 mg, about 20 mg, about 22.5 mg, or about 25 mg, and clorazepate is administered to a patient once a day, twice a day, three times per day, four times per day, five times per day, six times per day, seven times per day, eight times per day, nine times per day, or ten times per day. In some embodiments, the starting dose of clorazepate is 5 mg three times daily. In some embodiments, the starting dose of clorazepate is 10 mg three times daily. In some embodiments, the starting dose of clorazepate is 5 mg four times daily. In some embodiments, the starting dose of clorazepate is 10 mg four times daily. In some embodiments, the starting dose of clorazepate is 5 mg two times daily. In some embodiments, the starting dose of clorazepate is 5 mg four times daily. In some embodiments, the starting dose of clorazepate is 20 mg three times daily. In some embodiments, the starting dose of clorazepate is 20 mg four times daily. In some embodiments, the starting dose of clorazepate is 25 mg three times daily. In some embodiments, the starting dose of clorazepate is 25 mg four times daily. In some embodiments, the starting dose of clorazepate is the maintenance dose of clorazepate. In some embodiments, the starting dose of clorazepate is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg every day, every three to four days, every week, every other week, every third week, every fourth week, or every month until a maintenance dose is reached. In some embodiments, the starting dose of clorazepate is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is estazolam. In some embodiments, the starting dose of estazolam is between about 0.25 mg and about 1 mg of estazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of estazolam is between about 0.25 mg and about 1 mg of estazolam, for example about 0.25 mg, about 0.5 mg, about 0.75 mg, or about 1 mg, and the starting dose of estazolam is administered to a patient three times per day. In some embodiments, the starting dose of estazolam is the maintenance dose of estazolam. In some embodiments, the starting dose of estazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of estazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of estazolam is increased at three to four day intervals by no more than 1 mg/day.

In some embodiments, the benzodiazepine is flurazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg flurazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of flurazepam two to four times daily. In some embodiments, a patient is administered a starting dose of about 10 mg of flurazepam three to four times daily. In some embodiments, a patient is administered a starting dose of about 5 mg three times, or four times daily as needed. In some embodiments, the starting dose of flurazepam is the maintenance dose of flurazepam. In some embodiments, the starting dose of flurazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of flurazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is midazolam. In some embodiments, a starting dose of between about 0.125 and about 1 mg, for example, about 0.125 mg, about 0.25 mg, about 0.5 mg, about 0.75 mg, or about 1 mg, of midazolam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of midazolam is administered to a patient three times per day. In some embodiments, the starting dose of midazolam is the maintenance dose of midazolam. In some embodiments, the starting dose of midazolam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of midazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of midazolam is increased in increments of 0.125 to 0.25 mg twice a day every three days. In some embodiments, the maintenance dose of midazolam is 1 mg per day.

In some embodiments, the benzodiazepine is temazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg temazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of temazepam two to four times daily. In some embodiments, a patient is administered a starting dose of about 10 mg of temazepam three to four times daily. In some embodiments, a patient is administered a starting dose of about 5 mg three times, or four times daily as needed. In some embodiments, the starting dose of temazepam is the maintenance dose of temazepam. In some embodiments, the starting dose of temazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of temazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is triazolam. In some embodiments, the starting dose of triazolam is between about 0.25 mg and about 1 mg of triazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of triazolam is between about 0.25 mg and about 1 mg of triazolam, for example about 0.25 mg, about 0.5 mg, about 0.75 mg, or about 1 mg, and the starting dose of triazolam is administered to a patient three times per day. In some embodiments, the starting dose of triazolam is the maintenance dose of triazolam. In some embodiments, the starting dose of triazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of triazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of triazolam is increased at three to four day intervals by no more than 1 mg/day.

In some embodiments, the benzodiazepine is quazepam. In some embodiments, a starting dose of between about 0.125 and about 1 mg, for example, about 0.125 mg, about 0.25 mg, about 0.5 mg, about 0.75 mg, or about 1 mg, of quazepam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of quazepam is administered to a patient three times per day. In some embodiments, the starting dose of quazepam is the maintenance dose of quazepam. In some embodiments, the starting dose of quazepam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of quazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of quazepam is increased in increments of 0.125 to 0.25 mg twice a day every three days. In some embodiments, the maintenance dose of quazepam is 1 mg per day.

In some embodiments, a patient is administered a starting dose of oxazepam of between about 10 mg and about 30 mg, for example, about 10 mg, about 15 mg, about 20 mg, about 25 mg, or about 30 mg of oxazepam once a day, twice a day, three times per day, four times per day, five times per day, six times per day, seven times per day, eight times per day, nine times per day, or ten times per day. In some embodiments, a patient is administered a starting dose of oxazepam of about 10 mg to about 15 mg three to four times daily. In some embodiments, a patient is administered a starting dose of oxazepam of about 15 mg to about 30 mg three to four times daily. In some embodiments, a patient is administered a starting dose of oxazepam of about 10 mg three times daily. In some embodiments, a patient is administered a starting dose of oxazepam of about 15 mg three times daily. In some embodiments, a patient is administered a starting dose of oxazepam of about 15 mg four times daily. In some embodiments, the starting dose of oxazepam is the maintenance dose of oxazepam. In some embodiments, the starting dose of oxazepam is increased by between about 10 mg and about 30 mg, for example, about 10 mg, about 15 mg, about 20 mg, about 25 mg, or about 30 mg, every day, every three to four days, every week, every other week, every third week, every fourth week, or every month until a maintenance dose is reached. In some embodiments, the starting dose of oxazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

Administration of Starting Dose of Benzodiazepine During Administration of SSRI Maintenance Dose; SSRI administration is immediately ceased within 1-35 days of benzodiazepine administration

In some embodiments, a starting dose of a benzodiazepine is administered during an SSRI maintenance period but prior to administration of psilocybin. In some embodiments, administration of an SSRI is immediately stopped about 1 day to about 35 days after administration of a starting dose of benzodiazepine, including all subranges there between, for example, about 7 days to about 21 days, about 14 days to about 21 days, about 7 days to about 14 days, about 1 day to about 7 days, about 1 day to about 14 days, about 1 day to about 35 days, about 21 days to about 35 days, about 28 days to about 35 days, about 21 days to about 28 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, or about 35 days after administration of a starting dose of benzodiazepine. In some embodiments, a starting dose of a benzodiazepine is administered during administration of an SSRI maintenance dose but prior to administration of psilocybin, wherein the starting dose of a benzodiazepine is administered about 1 day to about 35 days before administration of psilocybin, including all subranges therebetween, for example, about 7 days to about 21 days, about 14 days to about 21 days, about 7 days to about 14 days, about 1 day to about 7 days, about 1 day to about 14 days, about 1 day to about 35 days, about 21 days to about 35 days, about 28 days to about 35 days, about 21 days to about 28 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, or about 35 days before administration of psilocybin.

In some embodiments, the starting dose of a benzodiazepine is the maintenance dose of a benzodiazepine. In some embodiments, the dose of a benzodiazepine is titrated from the starting dose to a maintenance dose.

In some embodiments, the benzodiazepine is alprazolam. In some embodiments, the starting dose of alprazolam is between about 0.25 mg and about 1 mg of alprazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of alprazolam is between about 0.25 mg and about 1 mg of alprazolam, for example about 0.25 mg, about 0.5 mg, about 0.75 mg, or about 1 mg, and the starting dose of alprazolam is administered to a patient three times per day. In some embodiments, the starting dose of alprazolam is the maintenance dose of alprazolam. In some embodiments, the starting dose of alprazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of alprazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of alprazolam is increased at three to four day intervals by no more than 1 mg/day.

In some embodiments, the benzodiazepine is chlordiazepoxide. In some embodiments, the starting dose of chlordiazepoxide is between about 5 mg and about 25 mg, for example, about 5 mg, about 7.5 mg, about 10 mg, about 12.5 mg, about 15 mg, about 17.5 mg, about 20 mg, about 22.5 mg, or about 25 mg, and chlordiazepoxide is administered to a patient once a day, twice a day, three times per day, four times per day, five times per day, six times per day, seven times per day, eight times per day, nine times per day, or ten times per day. In some embodiments, the starting dose of chlordiazepoxide is 5 mg three times daily. In some embodiments, the starting dose of chlordiazepoxide is 10 mg three times daily. In some embodiments, the starting dose of chlordiazepoxide is 5 mg four times daily. In some embodiments, the starting dose of chlordiazepoxide is 10 mg four times daily. In some embodiments, the starting dose of chlordiazepoxide is 5 mg two times daily. In some embodiments, the starting dose of chlordiazepoxide is 5 mg four times daily. In some embodiments, the starting dose of chlordiazepoxide is 20 mg three times daily. In some embodiments, the starting dose of chlordiazepoxide is 20 mg four times daily. In some embodiments, the starting dose of chlordiazepoxide is 25 mg three times daily. In some embodiments, the starting dose of chlordiazepoxide is 25 mg four times daily. In some embodiments, the starting dose of chlordiazepoxide is the maintenance dose of chlordiazepoxide. In some embodiments, the starting dose of chlordiazepoxide is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg every day, every three to four days, every week, every other week, every third week, every fourth week, or every month until a maintenance dose is reached. In some embodiments, the starting dose of chlordiazepoxide is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is clonazepam. In some embodiments, a starting dose of between about 0.125 and about 1 mg, for example, about 0.125 mg, about 0.25 mg, about 0.5 mg, about 0.75 mg, or about 1 mg, of clonazepam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of clonazepam is administered to a patient three times per day. In some embodiments, the starting dose of clonazepam is the maintenance dose of clonazepam. In some embodiments, the starting dose of clonazepam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of clonazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of clonazepam is increased in increments of 0.125 to 0.25 mg twice a day every three days. In some embodiments, the maintenance dose of clonazepam is 1 mg per day.

In some embodiments, the benzodiazepine is diazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg diazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of diazepam two to four times daily. In some embodiments, a patient is administered a starting dose of about 10 mg of diazepam three to four times daily. In some embodiments, a patient is administered a starting dose of about 5 mg three times, or four times daily as needed. In some embodiments, the starting dose of diazepam is the maintenance dose of diazepam. In some embodiments, the starting dose of diazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of diazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is lorazepam. In some embodiments, a patient is administered a starting dose of between about 0.5 mg and 6 mg of lorazepam, for example, about 0.5 mg, about 0.75 mg, about 1 mg, about 1.5 mg, about 2 mg, about 2.5 mg, about 3 mg, about 3.5 mg, about 4 mg, about 4.5 mg, about 5 mg, about 5.5 mg, or about 6 mg of lorazepam once a day, twice a day, three times per day, four times per day, five times per day, six times per day, seven times per day, eight times per day, nine times per day, or ten times per day. In some embodiments, a patient is administered a starting dose of lorazepam of between about 2 mg/day to 4 mg/day. In some embodiments, patients are administered a starting dose of between about 2 mg/day to 3 mg/day given two times a day or three times a day. In some embodiments, the benzodiazepine is lorazepam. In some embodiments, the starting dose of lorazepam is the maintenance dose of lorazepam. In some embodiments, the starting dose of lorazepam is increased by between about 0.5 mg and 6 mg of lorazepam, for example, about 0.5 mg, about 0.75 mg, about 1 mg, about 1.5 mg, about 2 mg, about 2.5 mg, about 3 mg, about 3.5 mg, about 4 mg, about 4.5 mg, about 5 mg, about 5.5 mg, or about 6 mg, every day, every three to four days, every week, every other week, every third week, every fourth week, or every month until a maintenance dose is reached. In some embodiments, the starting dose of lorazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is clorazepate. In some embodiments, the starting dose of clorazepate is between about 5 mg and about 25 mg, for example, about 5 mg, about 7.5 mg, about 10 mg, about 12.5 mg, about 15 mg, about 17.5 mg, about 20 mg, about 22.5 mg, or about 25 mg, and clorazepate is administered to a patient once a day, twice a day, three times per day, four times per day, five times per day, six times per day, seven times per day, eight times per day, nine times per day, or ten times per day. In some embodiments, the starting dose of clorazepate is 5 mg three times daily. In some embodiments, the starting dose of clorazepate is 10 mg three times daily. In some embodiments, the starting dose of clorazepate is 5 mg four times daily. In some embodiments, the starting dose of clorazepate is 10 mg four times daily. In some embodiments, the starting dose of clorazepate is 5 mg two times daily. In some embodiments, the starting dose of clorazepate is 5 mg four times daily. In some embodiments, the starting dose of clorazepate is 20 mg three times daily. In some embodiments, the starting dose of clorazepate is 20 mg four times daily. In some embodiments, the starting dose of clorazepate is 25 mg three times daily. In some embodiments, the starting dose of clorazepate is 25 mg four times daily. In some embodiments, the starting dose of clorazepate is the maintenance dose of clorazepate. In some embodiments, the starting dose of clorazepate is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg every day, every three to four days, every week, every other week, every third week, every fourth week, or every month until a maintenance dose is reached. In some embodiments, the starting dose of clorazepate is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is estazolam. In some embodiments, the starting dose of estazolam is between about 0.25 mg and about 1 mg of estazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of estazolam is between about 0.25 mg and about 1 mg of estazolam, for example about 0.25 mg, about 0.5 mg, about 0.75 mg, or about 1 mg, and the starting dose of estazolam is administered to a patient three times per day. In some embodiments, the starting dose of estazolam is the maintenance dose of estazolam. In some embodiments, the starting dose of estazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of estazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of estazolam is increased at three to four day intervals by no more than 1 mg/day.

In some embodiments, the benzodiazepine is flurazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg flurazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of flurazepam two to four times daily. In some embodiments, a patient is administered a starting dose of about 10 mg of flurazepam three to four times daily. In some embodiments, a patient is administered a starting dose of about 5 mg three times, or four times daily as needed. In some embodiments, the starting dose of flurazepam is the maintenance dose of flurazepam. In some embodiments, the starting dose of flurazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of flurazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is midazolam. In some embodiments, a starting dose of between about 0.125 and about 1 mg, for example, about 0.125 mg, about 0.25 mg, about 0.5 mg, about 0.75 mg, or about 1 mg, of midazolam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of midazolam is administered to a patient three times per day. In some embodiments, the starting dose of midazolam is the maintenance dose of midazolam. In some embodiments, the starting dose of midazolam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of midazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of midazolam is increased in increments of 0.125 to 0.25 mg twice a day every three days. In some embodiments, the maintenance dose of midazolam is 1 mg per day.

In some embodiments, the benzodiazepine is temazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg temazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of temazepam two to four times daily. In some embodiments, a patient is administered a starting dose of about 10 mg of temazepam three to four times daily. In some embodiments, a patient is administered a starting dose of about 5 mg three times, or four times daily as needed. In some embodiments, the starting dose of temazepam is the maintenance dose of temazepam. In some embodiments, the starting dose of temazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of temazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is triazolam. In some embodiments, the starting dose of triazolam is between about 0.25 mg and about 1 mg of triazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of triazolam is between about 0.25 mg and about 1 mg of triazolam, for example about 0.25 mg, about 0.5 mg, about 0.75 mg, or about 1 mg, and the starting dose of triazolam is administered to a patient three times per day. In some embodiments, the starting dose of triazolam is the maintenance dose of triazolam. In some embodiments, the starting dose of triazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of triazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of triazolam is increased at three to four day intervals by no more than 1 mg/day.

In some embodiments, the benzodiazepine is quazepam. In some embodiments, a starting dose of between about 0.125 and about 1 mg, for example, about 0.125 mg, about 0.25 mg, about 0.5 mg, about 0.75 mg, or about 1 mg, of quazepam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of quazepam is administered to a patient three times per day. In some embodiments, the starting dose of quazepam is the maintenance dose of quazepam. In some embodiments, the starting dose of quazepam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of quazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of quazepam is increased in increments of 0.125 to 0.25 mg twice a day every three days. In some embodiments, the maintenance dose of quazepam is 1 mg per day.

In some embodiments, a patient is administered a starting dose of oxazepam of between about 10 mg and about 30 mg, for example, about 10 mg, about 15 mg, about 20 mg, about 25 mg, or about 30 mg of oxazepam once a day, twice a day, three times per day, four times per day, five times per day, six times per day, seven times per day, eight times per day, nine times per day, or ten times per day. In some embodiments, a patient is administered a starting dose of oxazepam of about 10 mg to about 15 mg three to four times daily. In some embodiments, a patient is administered a starting dose of oxazepam of about 15 mg to about 30 mg three to four times daily. In some embodiments, a patient is administered a starting dose of oxazepam of about 10 mg three times daily. In some embodiments, a patient is administered a starting dose of oxazepam of about 15 mg three times daily. In some embodiments, a patient is administered a starting dose of oxazepam of about 15 mg four times daily. In some embodiments, the starting dose of oxazepam is the maintenance dose of oxazepam. In some embodiments, the starting dose of oxazepam is increased by between about 10 mg and about 30 mg, for example, about 10 mg, about 15 mg, about 20 mg, about 25 mg, or about 30 mg, every day, every three to four days, every week, every other week, every third week, every fourth week, or every month until a maintenance dose is reached. In some embodiments, the starting dose of oxazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

### Administration of Starting Dose of Benzodiazepine During Titration Period of SSRI

In some embodiments, a starting dose of a benzodiazepine is administered during a titration period of an SSRI, wherein during the titration period of an SSRI, the SSRI is titrated from a maintenance dose to cessation. In some embodiments, the titration period of an SSRI is about 1 day to about 35 days, for example, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, or about 35 days. In some embodiments, the titration period is about 1 day to about 3 months, for example, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 6 weeks, about 7 weeks, about 8 weeks, about 9 weeks, about 10 weeks, about 11 weeks, about 12 weeks, about 13 weeks, about 14 weeks, about 15 weeks, about 1 month, about 2 months, or about 3 months. In some embodiments, the first day of the titration period of SSRI is the day after the final maintenance dose of SSRI is administered. In some embodiments, a starting dose of a benzodiazepine is administered during a titration period of an SSRI, wherein during the titration period of an SSRI, the SSRI is titrated from a maintenance dose to cessation and wherein the benzodiazepine is administered prior to psilocybin administration. In some embodiments, a starting dose of a benzodiazepine is administered during a titration period of an SSRI, wherein during the titration period of an SSRI, the SSRI is titrated from a maintenance dose to cessation, and wherein the benzodiazepine is administered about 1 day to about 21 days prior to psilocybin administration, including all subranges there between, for example, about 7 days to about 21 days, about 14 days to about 21 days, about 7 days to about 14 days, about 1 day to about 7 days, about 1 day to about 14 days, about 1 day to about 21 days, about 1 day to about 28 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, or about 21 days administration of psilocybin.

In some embodiments, the starting dose of a benzodiazepine is the maintenance dose of a benzodiazepine. In some embodiments, the dose of a benzodiazepine is titrated from the starting dose to a maintenance dose.

In some embodiments, the benzodiazepine is alprazolam. In some embodiments, the starting dose of alprazolam is between about 0.25 mg and about 1 mg of alprazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of alprazolam is between about 0.25 mg and about 1 mg of alprazolam, for example about 0.25 mg, about 0.5 mg, about 0.75 mg, or about 1 mg, and the starting dose of alprazolam is administered to a patient three times per day. In some embodiments, the starting dose of alprazolam is the maintenance dose of alprazolam. In some embodiments, the starting dose of alprazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of alprazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of alprazolam is increased at three to four day intervals by no more than 1 mg/day.

In some embodiments, the benzodiazepine is chlordiazepoxide. In some embodiments, the starting dose of chlordiazepoxide is between about 5 mg and about 25 mg, for example, about 5 mg, about 7.5 mg, about 10 mg, about 12.5 mg, about 15 mg, about 17.5 mg, about 20 mg, about 22.5 mg, or about 25 mg, and chlordiazepoxide is administered to a patient once a day, twice a day, three times per day, four times per day, five times per day, six times per day, seven times per day, eight times per day, nine times per day, or ten times per day. In some embodiments, the starting dose of chlordiazepoxide is 5 mg three times daily. In some embodiments, the starting dose of chlordiazepoxide is 10 mg three times daily. In some embodiments, the starting dose of chlordiazepoxide is 5 mg four times daily. In some embodiments, the starting dose of chlordiazepoxide is 10 mg four times daily. In some embodiments, the starting dose of chlordiazepoxide is 5 mg two times daily. In some embodiments, the starting dose of chlordiazepoxide is 5 mg four times daily. In some embodiments, the starting dose of chlordiazepoxide is 20 mg three times daily. In some embodiments, the starting dose of chlordiazepoxide is 20 mg four times daily. In some embodiments, the starting dose of chlordiazepoxide is 25 mg three times daily. In some embodiments, the starting dose of chlordiazepoxide is 25 mg four times daily. In some embodiments, the starting dose of chlordiazepoxide is the maintenance dose of chlordiazepoxide. In some embodiments, the starting dose of chlordiazepoxide is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg every day, every three to four days, every week, every other week, every third week, every fourth week, or every month until a maintenance dose is reached. In some embodiments, the starting dose of chlordiazepoxide is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is clonazepam. In some embodiments, a starting dose of between about 0.125 and about 1 mg, for example, about 0.125 mg, about 0.25 mg, about 0.5 mg, about 0.75 mg, or about 1 mg, of clonazepam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of clonazepam is administered to a patient three times per day. In some embodiments, the starting dose of clonazepam is the maintenance dose of clonazepam. In some embodiments, the starting dose of clonazepam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of clonazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of clonazepam is increased in increments of 0.125 to 0.25 mg twice a day every three days. In some embodiments, the maintenance dose of clonazepam is 1 mg per day.

In some embodiments, the benzodiazepine is diazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg diazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of diazepam two to four times daily. In some embodiments, a patient is administered a starting dose of about 10 mg of diazepam three to four times daily. In some embodiments, a patient is administered a starting dose of about 5 mg three times, or four times daily as needed. In some embodiments, the starting dose of diazepam is the maintenance dose of diazepam. In some embodiments, the starting dose of diazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of diazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is lorazepam. In some embodiments, a patient is administered a starting dose of between about 0.5 mg and 6 mg of lorazepam, for example, about 0.5 mg, about 0.75 mg, about 1 mg, about 1.5 mg, about 2 mg, about 2.5 mg, about 3 mg, about 3.5 mg, about 4 mg, about 4.5 mg, about 5 mg, about 5.5 mg, or about 6 mg of lorazepam once a day, twice a day, three times per day, four times per day, five times per day, six times per day, seven times per day, eight times per day, nine times per day, or ten times per day. In some embodiments, a patient is administered a starting dose of lorazepam of between about 2 mg/day to 4 mg/day. In some embodiments, patients are administered a starting dose of between about 2 mg/day to 3 mg/day given two times a day or three times a day. In some embodiments, the benzodiazepine is lorazepam. In some embodiments, the starting dose of lorazepam is the maintenance dose of lorazepam. In some embodiments, the starting dose of lorazepam is increased by between about 0.5 mg and 6 mg of lorazepam, for example, about 0.5 mg, about 0.75 mg, about 1 mg, about 1.5 mg, about 2 mg, about 2.5 mg, about 3 mg, about 3.5 mg, about 4 mg, about 4.5 mg, about 5 mg, about 5.5 mg, or about 6 mg, every day, every three to four days, every week, every other week, every third week, every fourth week, or every month until a maintenance dose is reached. In some embodiments, the starting dose of lorazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is clorazepate. In some embodiments, the starting dose of clorazepate is between about 5 mg and about 25 mg, for example, about 5 mg, about 7.5 mg, about 10 mg, about 12.5 mg, about 15 mg, about 17.5 mg, about 20 mg, about 22.5 mg, or about 25 mg, and clorazepate is administered to a patient once a day, twice a day, three times per day, four times per day, five times per day, six times per day, seven times per day, eight times per day, nine times per day, or ten times per day. In some embodiments, the starting dose of clorazepate is 5 mg three times daily. In some embodiments, the starting dose of clorazepate is 10 mg three times daily. In some embodiments, the starting dose of clorazepate is 5 mg four times daily. In some embodiments, the starting dose of clorazepate is 10 mg four times daily. In some embodiments, the starting dose of clorazepate is 5 mg two times daily. In some embodiments, the starting dose of clorazepate is 5 mg four times daily. In some embodiments, the starting dose of clorazepate is 20 mg three times daily. In some embodiments, the starting dose of clorazepate is 20 mg four times daily. In some embodiments, the starting dose of clorazepate is 25 mg three times daily. In some embodiments, the starting dose of clorazepate is 25 mg four times daily. In some embodiments, the starting dose of clorazepate is the maintenance dose of clorazepate. In some embodiments, the starting dose of clorazepate is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg every day, every three to four days, every week, every other week, every third week, every fourth week, or every month until a maintenance dose is reached. In some embodiments, the starting dose of clorazepate is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is estazolam. In some embodiments, the starting dose of estazolam is between about 0.25 mg and about 1 mg of estazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of estazolam is between about 0.25 mg and about 1 mg of estazolam, for example about 0.25 mg, about 0.5 mg, about 0.75 mg, or about 1 mg, and the starting dose of estazolam is administered to a patient three times per day. In some embodiments, the starting dose of estazolam is the maintenance dose of estazolam. In some embodiments, the starting dose of estazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of estazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of estazolam is increased at three to four day intervals by no more than 1 mg/day.

In some embodiments, the benzodiazepine is flurazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg flurazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of flurazepam two to four times daily. In some embodiments, a patient is administered a starting dose of about 10 mg of flurazepam three to four times daily. In some embodiments, a patient is administered a starting dose of about 5 mg three times, or four times daily as needed. In some embodiments, the starting dose of flurazepam is the maintenance dose of flurazepam. In some embodiments, the starting dose of flurazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of flurazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is midazolam. In some embodiments, a starting dose of between about 0.125 and about 1 mg, for example, about 0.125 mg, about 0.25 mg, about 0.5 mg, about 0.75 mg, or about 1 mg, of midazolam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of midazolam is administered to a patient three times per day. In some embodiments, the starting dose of midazolam is the maintenance dose of midazolam. In some embodiments, the starting dose of midazolam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of midazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of midazolam is increased in increments of 0.125 to 0.25 mg twice a day every three days. In some embodiments, the maintenance dose of midazolam is 1 mg per day.

In some embodiments, the benzodiazepine is temazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg temazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of temazepam two to four times daily. In some embodiments, a patient is administered a starting dose of about 10 mg of temazepam three to four times daily. In some embodiments, a patient is administered a starting dose of about 5 mg three times, or four times daily as needed. In some embodiments, the starting dose of temazepam is the maintenance dose of temazepam. In some embodiments, the starting dose of temazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of temazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is triazolam. In some embodiments, the starting dose of triazolam is between about 0.25 mg and about 1 mg of triazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of triazolam is between about 0.25 mg and about 1 mg of triazolam, for example about 0.25 mg, about 0.5 mg, about 0.75 mg, or about 1 mg, and the starting dose of triazolam is administered to a patient three times per day. In some embodiments, the starting dose of triazolam is the maintenance dose of triazolam. In some embodiments, the starting dose of triazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of triazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of triazolam is increased at three to four day intervals by no more than 1 mg/day.

In some embodiments, the benzodiazepine is quazepam. In some embodiments, a starting dose of between about 0.125 and about 1 mg, for example, about 0.125 mg, about 0.25 mg, about 0.5 mg, about 0.75 mg, or about 1 mg, of quazepam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of quazepam is administered to a patient three times per day. In some embodiments, the starting dose of quazepam is the maintenance dose of quazepam. In some embodiments, the starting dose of quazepam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of quazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of quazepam is increased in increments of 0.125 to 0.25 mg twice a day every three days. In some embodiments, the maintenance dose of quazepam is 1 mg per day.

In some embodiments, a patient is administered a starting dose of oxazepam of between about 10 mg and about 30 mg, for example, about 10 mg, about 15 mg, about 20 mg, about 25 mg, or about 30 mg of oxazepam once a day, twice a day, three times per day, four times per day, five times per day, six times per day, seven times per day, eight times per day, nine times per day, or ten times per day. In some embodiments, a patient is administered a starting dose of oxazepam of about 10 mg to about 15 mg three to four times daily. In some embodiments, a patient is administered a starting dose of oxazepam of about 15 mg to about 30 mg three to four times daily. In some embodiments, a patient is administered a starting dose of oxazepam of about 10 mg three times daily. In some embodiments, a patient is administered a starting dose of oxazepam of about 15 mg three times daily. In some embodiments, a patient is administered a starting dose of oxazepam of about 15 mg four times daily. In some embodiments, the starting dose of oxazepam is the maintenance dose of oxazepam. In some embodiments, the starting dose of oxazepam is increased by between about 10 mg and about 30 mg, for example, about 10 mg, about 15 mg, about 20 mg, about 25 mg, or about 30 mg, every day, every three to four days, every week, every other week, every third week, every fourth week, or every month until a maintenance dose is reached. In some embodiments, the starting dose of oxazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

Administration of Starting Dose of Benzodiazepine During Maintenance Period of SSRI where SSRI is Titrated to Cessation During Titration Period

In some embodiments, a starting dose of a benzodiazepine is administered during a maintenance period of an SSRI but prior to a titration period of an SSRI, wherein during the titration period of an SSRI, the SSRI is titrated from a maintenance dose to cessation, and wherein the starting dose of benzodiazepine is administered prior to administration of psilocybin. In some embodiments, a starting dose of a benzodiazepine is administered during a maintenance period of an SSRI but prior to a titration period of an SSRI, wherein during the titration period of an SSRI, the SSRI is titrated from a maintenance dose to cessation, and wherein the starting dose of benzodiazepine is administered about 1 day to about 35 days prior to administration of psilocybin, including all subranges therebetween, for example, about 7 days to about 21 days, about 14 days to about 21 days, about 7 days to about 14 days, about 1 day to about 7 days, about 1 day to about 14 days, about 1 day to about 35 days, about 21 days to about 35 days, about 28 days to about 35 days, about 21 days to about 28 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, or about 35 days before administration of psilocybin.

In some embodiments, the starting dose of a benzodiazepine is the maintenance dose of a benzodiazepine. In some embodiments, the dose of a benzodiazepine is titrated from the starting dose to a maintenance dose.

In some embodiments, the benzodiazepine is alprazolam. In some embodiments, the starting dose of alprazolam is between about 0.25 mg and about 1 mg of alprazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of alprazolam is between about 0.25 mg and about 1 mg of alprazolam, for example about 0.25 mg, about 0.5 mg, about 0.75 mg, or about 1 mg, and the starting dose of alprazolam is administered to a patient three times per day. In some embodiments, the starting dose of alprazolam is the maintenance dose of alprazolam. In some embodiments, the starting dose of alprazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of alprazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of alprazolam is increased at three to four day intervals by no more than 1 mg/day.

In some embodiments, the benzodiazepine is chlordiazepoxide. In some embodiments, the starting dose of chlordiazepoxide is between about 5 mg and about 25 mg, for example, about 5 mg, about 7.5 mg, about 10 mg, about 12.5 mg, about 15 mg, about 17.5 mg, about 20 mg, about 22.5 mg, or about 25 mg, and chlordiazepoxide is administered to a patient once a day, twice a day, three times per day, four times per day, five times per day, six times per day, seven times per day, eight times per day, nine times per day, or ten times per day. In some embodiments, the starting dose of chlordiazepoxide is 5 mg three times daily. In some embodiments, the starting dose of chlordiazepoxide is 10 mg three times daily. In some embodiments, the starting dose of chlordiazepoxide is 5 mg four times daily. In some embodiments, the starting dose of chlordiazepoxide is 10 mg four times daily. In some embodiments, the starting dose of chlordiazepoxide is 5 mg two times daily. In some embodiments, the starting dose of chlordiazepoxide is 5 mg four times daily. In some embodiments, the starting dose of chlordiazepoxide is 20 mg three times daily. In some embodiments, the starting dose of chlordiazepoxide is 20 mg four times daily. In some embodiments, the starting dose of chlordiazepoxide is 25 mg three times daily. In some embodiments, the starting dose of chlordiazepoxide is 25 mg four times daily. In some embodiments, the starting dose of chlordiazepoxide is the maintenance dose of chlordiazepoxide. In some embodiments, the starting dose of chlordiazepoxide is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg every day, every three to four days, every week, every other week, every third week, every fourth week, or every month until a maintenance dose is reached. In some embodiments, the starting dose of chlordiazepoxide is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is clonazepam. In some embodiments, a starting dose of between about 0.125 and about 1 mg, for example, about 0.125 mg, about 0.25 mg, about 0.5 mg, about 0.75 mg, or about 1 mg, of clonazepam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of clonazepam is administered to a patient three times per day. In some embodiments, the starting dose of clonazepam is the maintenance dose of clonazepam. In some embodiments, the starting dose of clonazepam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of clonazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of clonazepam is increased in increments of 0.125 to 0.25 mg twice a day every three days. In some embodiments, the maintenance dose of clonazepam is 1 mg per day.

In some embodiments, the benzodiazepine is diazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg diazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of diazepam two to four times daily. In some embodiments, a patient is administered a starting dose of about 10 mg of diazepam three to four times daily. In some embodiments, a patient is administered a starting dose of about 5 mg three times, or four times daily as needed. In some embodiments, the starting dose of diazepam is the maintenance dose of diazepam. In some embodiments, the starting dose of diazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of diazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is lorazepam. In some embodiments, a patient is administered a starting dose of between about 0.5 mg and 6 mg of lorazepam, for example, about 0.5 mg, about 0.75 mg, about 1 mg, about 1.5 mg, about 2 mg, about 2.5 mg, about 3 mg, about 3.5 mg, about 4 mg, about 4.5 mg, about 5 mg, about 5.5 mg, or about 6 mg of lorazepam once a day, twice a day, three times per day, four times per day, five times per day, six times per day, seven times per day, eight times per day, nine times per day, or ten times per day. In some embodiments, a patient is administered a starting dose of lorazepam of between about 2 mg/day to 4 mg/day. In some embodiments, patients are administered a starting dose of between about 2 mg/day to 3 mg/day given two times a day or three times a day. In some embodiments, the benzodiazepine is lorazepam. In some embodiments, the starting dose of lorazepam is the maintenance dose of lorazepam. In some embodiments, the starting dose of lorazepam is increased by between about 0.5 mg and 6 mg of lorazepam, for example, about 0.5 mg, about 0.75 mg, about 1 mg, about 1.5 mg, about 2 mg, about 2.5 mg, about 3 mg, about 3.5 mg, about 4 mg, about 4.5 mg, about 5 mg, about 5.5 mg, or about 6 mg, every day, every three to four days, every week, every other week, every third week, every fourth week, or every month until a maintenance dose is reached. In some embodiments, the starting dose of lorazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is clorazepate. In some embodiments, the starting dose of clorazepate is between about 5 mg and about 25 mg, for example, about 5 mg, about 7.5 mg, about 10 mg, about 12.5 mg, about 15 mg, about 17.5 mg, about 20 mg, about 22.5 mg, or about 25 mg, and clorazepate is administered to a patient once a day, twice a day, three times per day, four times per day, five times per day, six times per day, seven times per day, eight times per day, nine times per day, or ten times per day. In some embodiments, the starting dose of clorazepate is 5 mg three times daily. In some embodiments, the starting dose of clorazepate is 10 mg three times daily. In some embodiments, the starting dose of clorazepate is 5 mg four times daily. In some embodiments, the starting dose of clorazepate is 10 mg four times daily. In some embodiments, the starting dose of clorazepate is 5 mg two times daily. In some embodiments, the starting dose of clorazepate is 5 mg four times daily. In some embodiments, the starting dose of clorazepate is 20 mg three times daily. In some embodiments, the starting dose of clorazepate is 20 mg four times daily. In some embodiments, the starting dose of clorazepate is 25 mg three times daily. In some embodiments, the starting dose of clorazepate is 25 mg four times daily. In some embodiments, the starting dose of clorazepate is the maintenance dose of clorazepate. In some embodiments, the starting dose of clorazepate is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg every day, every three to four days, every week, every other week, every third week, every fourth week, or every month until a maintenance dose is reached. In some embodiments, the starting dose of clorazepate is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is estazolam. In some embodiments, the starting dose of estazolam is between about 0.25 mg and about 1 mg of estazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of estazolam is between about 0.25 mg and about 1 mg of estazolam, for example about 0.25 mg, about 0.5 mg, about 0.75 mg, or about 1 mg, and the starting dose of estazolam is administered to a patient three times per day. In some embodiments, the starting dose of estazolam is the maintenance dose of estazolam. In some embodiments, the starting dose of estazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of estazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of estazolam is increased at three to four day intervals by no more than 1 mg/day.

In some embodiments, the benzodiazepine is flurazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg flurazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of flurazepam two to four times daily. In some embodiments, a patient is administered a starting dose of about 10 mg of flurazepam three to four times daily. In some embodiments, a patient is administered a starting dose of about 5 mg three times, or four times daily as needed. In some embodiments, the starting dose of flurazepam is the maintenance dose of flurazepam. In some embodiments, the starting dose of flurazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of flurazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is midazolam. In some embodiments, a starting dose of between about 0.125 and about 1 mg, for example, about 0.125 mg, about 0.25 mg, about 0.5 mg, about 0.75 mg, or about 1 mg, of midazolam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of midazolam is administered to a patient three times per day. In some embodiments, the starting dose of midazolam is the maintenance dose of midazolam. In some embodiments, the starting dose of midazolam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of midazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of midazolam is increased in increments of 0.125 to 0.25 mg twice a day every three days. In some embodiments, the maintenance dose of midazolam is 1 mg per day.

In some embodiments, the benzodiazepine is temazepam. In some embodiments, a patient is administered a starting dose of between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg temazepam once a day, twice a day, three times per day, four times per day, five times per day, six times per day, seven times per day, eight times per day, nine times per day, or ten times per day. In some embodiments, a patient is administered a starting dose of between about 2 mg and about 10 mg of temazepam two to four times daily. In some embodiments, a patient is administered a starting dose of about 10 mg of temazepam three to four times daily. In some embodiments, a patient is administered a starting dose of about 5 mg three times, or four times daily as needed. In some embodiments, the starting dose of temazepam is the maintenance dose of temazepam. In some embodiments, the starting dose of temazepam is increased by between about 1 mg and about 25 mg, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg, every day, every three to four days, every week, every other week, every third week, every fourth week, or every month until a maintenance dose is reached. In some embodiments, the starting dose of temazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

In some embodiments, the benzodiazepine is triazolam. In some embodiments, the starting dose of triazolam is between about 0.25 mg and about 1 mg of triazolam, for example about 0.25 mg, about 0.5 mg, about 0.75 mg, or about 1 mg, and the starting dose is administered to a patient once a day, twice a day, three times per day, four times per day, five times per day, six times per day, seven times per day, eight times per day, nine times per day, or ten times per day. In some embodiments, the starting dose of triazolam is between about 0.25 mg and about 1 mg of triazolam, for example about 0.25 mg, about 0.5 mg, about 0.75 mg, or about 1 mg, and the starting dose of triazolam is administered to a patient three times per day. In some embodiments, the starting dose of triazolam is the maintenance dose of triazolam. In some embodiments, the starting dose of triazolam is increased by between about 0.25 mg and about 1 mg, for example, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of triazolam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of triazolam is increased at three to four day intervals by no more than 1 mg/day.

In some embodiments, the benzodiazepine is quazepam. In some embodiments, a starting dose of between about 0.125 and about 1 mg, for example, about 0.125 mg, about 0.25 mg, about 0.5 mg, about 0.75 mg, or about 1 mg, of quazepam is administered to a patient once a day, twice a day, three times per day, four times per day, five times per day, six times per day, seven times per day, eight times per day, nine times per day, or ten times per day. In some embodiments, a starting dose of between about 0.125 and about 1 mg, for example, about 0.125 mg, about 0.25 mg, about 0.5 mg, about 0.75 mg, or about 1 mg, of quazepam is administered to a patient three times per day. In some embodiments, the starting dose of quazepam is the maintenance dose of quazepam. In some embodiments, the starting dose of quazepam is increased by between about 0.125 mg and about 1 mg, for example, about 0.125 mg, about 0.25 mg, about 0.5 mg, about 0.75 mg, or about 1 mg, every day, every three to four days, every week, every other week, every third week, every fourth week, or every month until a maintenance dose is reached. In some embodiments, the starting dose of quazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved. In some embodiments, the starting dose of quazepam is increased in increments of 0.125 to 0.25 mg twice a day every three days. In some embodiments, the maintenance dose of quazepam is 1 mg per day.

In some embodiments, a patient is administered a starting dose of oxazepam of between about 10 mg and about 30 mg, for example, about 10 mg, about 15 mg, about 20 mg, about 25 mg, or about 30 mg of oxazepam once a day, twice a day, three times per day, four times per day, five times per day, six times per day, seven times per day, eight times per day, nine times per day, or ten times per day. In some embodiments, a patient is administered a starting dose of oxazepam of about 10 mg to about 15 mg three to four times daily. In some embodiments, a patient is administered a starting dose of oxazepam of about 15 mg to about 30 mg three to four times daily. In some embodiments, a patient is administered a starting dose of oxazepam of about 10 mg three times daily. In some embodiments, a patient is administered a starting dose of oxazepam of about 15 mg three times daily. In some embodiments, a patient is administered a starting dose of oxazepam of about 15 mg four times daily. In some embodiments, the starting dose of oxazepam is the maintenance dose of oxazepam. In some embodiments, the starting dose of oxazepam is increased by between about 10 mg and about 30 mg, for example, about 10 mg, about 15 mg, about 20 mg, about 25 mg, or about 30 mg, every day, every three to four days, every week, every other week, every third week, every fourth week, or every month until a maintenance dose is reached. In some embodiments, the starting dose of oxazepam is increased from the starting dose by about 50% to about 500% and all subranges there between, for example, about 50% to about 100%, about 50% to about 200%, about 50% to 300%, about 50% to about 400%, about 100% to about 200%, about 100% to about 300%, about 100% to about 400%, about 100% to about 500%, about 200% to about 300%, about 200% to about 400%, about 200% to about 500%, about 300% to about 400%, about 300% to about 500%, about 400% to about 500%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500% every day, every three to four days, every week, every other week, every three weeks, every four weeks, or every month until a maintenance dose has been achieved.

### Administration Routes and Methods

Exemplary modes for administration of psilocybin and/or a benzodiazepine and/or a SSRI include oral, parenteral (e.g., intravenous, subcutaneous, intradermal, intramuscular [including administration to skeletal, diaphragm and/or cardiac muscle], intradermal, intrapleural, intracerebral, and intra-articular), topical (e.g., to both skin and mucosal surfaces, including airway surfaces, and transdermal administration), inhalation (e.g., via an aerosol), rectal, transmucosal, intranasal, buccal (e.g., sublingual), vaginal, intrathecal, intraocular, transdermal, in utero (or in ovo), intralymphatic, and direct tissue or organ injection (e.g., to liver, skeletal muscle, cardiac muscle, diaphragm muscle or brain). In some embodiments, psilocybin is administered orally to the patient.

Psilocybin and/or a benzodiazepine and/or a SSRI may be administered to a patient in the patient's home or at a clinical facility. In some embodiments, the patient is supervised during the administration. In some embodiments, the patient is supervised during the administration and for a period of time thereafter (e.g., at least 4 to 12 hours thereafter). In some embodiments, the supervision is performed by at least one professional who has been trained to administer psilocybin therapy. In some embodiments, the professional is a doctor, a nurse, a psychotherapist, a counselor, or other mental health professional.

In some embodiments, the patient receives psychological support during the administration, and for at least 4 to 12 hours thereafter. In some embodiments, the psychological support is provided by at least one professional who has been trained to administer psilocybin therapy. In some embodiments, the professional is a doctor, a nurse, a psychotherapist, a counselor, or other mental health professional.

In some embodiments, the patient receives counseling with regard to the expected effects of the psilocybin before the psilocybin is administered to the patient. In some embodiments, the counseling is provided by at least one professional who has been trained to administer psilocybin therapy. In some embodiments, the professional is a doctor, a nurse, a psychotherapist, a counselor, or other mental health professional.

### Patients

In some embodiments, the patient is a male. In some embodiments, the patient is a female. In some embodiments, the female patient is pregnant or post-partum. In some embodiments, the patient is attempting to reduce or eliminate their use of a pharmaceutical agent, such as an anti-depressant or an anti-epileptic drug. In some embodiments, the patient is attempting to reduce or eliminate their use of the pharmaceutical agent before becoming pregnant, having surgery or other medical procedure, or starting to use different pharmaceutical agent.

The patient may be a geriatric patient, a pediatric patient, a teenage patient, a young adult patient, or a middle aged patient. In some embodiments, the patient is less than about 18 years of age. In some embodiments, the patient is at least about 18 years of age. In some embodiments, the patient is about 5-10, about 10-15, about 15-20, about 20-25, about 25-30, about 30-35, about 35-40, about 40-45, about 45-50, about 50-55, about 55-60, about 60-65, about 65-70, about 70-75, about 75-80, about 85-90, about 90-95, or about 95-100 years of age.

The patient may have a chronic disease or a terminal disease. In some embodiments, the patient may have a life-altering disease or condition (such as the loss of a limb or onset of blindness).

The patient may have recently been diagnosed with a disease, disorder, or condition. For example, the patient may have been diagnosed within 1 month, within 3 months, within 6 months, or within 1 year. In some embodiments, the patient may have been living with a disease, disorder, or condition for an extended period time, such as at least 6 months, at least 1 year, at least 3 years, at least 5 years, or at least 10 years.

In some embodiments, the patient may be a cancer patient, such as a Stage 4 or terminal cancer patient. In some embodiments, the patient may have been determined to have a limited time to live, such as less than 1 year, less than 6 months, or less than 3 months.

The patient may have previously taken a psychedelic drug or may have never previously taken a psychedelic drug. For example, the patient may or may not have previously taken psilocybin, a psilocybin mushroom (“magic mushroom”), LSD (lysergic acid diethylamide or acid), mescaline, or DMT (N,N-Dimethyltryptamine).

In some embodiments, the patient may have previously taken one or more serotonergic antidepressants (e.g., selective serotonin reuptake inhibitors (SSRIs). In some embodiments, the patient has never previously taken a serotonergic antidepressant. In some embodiments, the patient has not taken any serotergic antidepressants for at least 2 weeks, at least 4 weeks, or at least 6 weeks prior to receiving psilocybin.

In some embodiments, the patient may have previously received electroconvulsive therapy (ECT). In some embodiments, the patient has not received any ECT for at least 2 weeks, at least 4 weeks, or at least 6 weeks prior to receiving psilocybin.

The patient may have a medical condition that prevents the patient from receiving a particular medical therapy (such as an SSRI or ECT). In some embodiments, the patient may have previously had an adverse reaction to a particular medical therapy (such as an SSRI or ECT). In some embodiments, a prior medical therapy (such as an SSRI or ECT) was not effective in treating a disease, disorder, or condition in the patient.

In some embodiments, the patient is administered an SSRI regimen prior to administration of psilocybin. In some embodiments, the SSRI regimen is administered chronically. As used herein, chronic administration of an SSRI regimen refers to administration of an SSRI for at least 4 weeks, for example, about 4 weeks, about 5 weeks, about 6 weeks, 7 weeks, 8 weeks, 9 weeks, 10 weeks, 11 weeks, 12 weeks, 3 months, 4 months, 5 months, 6 months, 7 months, 8 months, 9 months, 10 months, 11 months, 12 months, or more. In some embodiments, chronic administration of an SSRI regiment is administration of an SSRI for at least 6 weeks.

In some embodiments, the patient is suffering from one or more of the following diseases or disorders: Disruptive Mood Dysregulation Disorder, Major Depressive Disorder (MDD), Treatment-Resistant Depression, Persistent Depressive Disorder (Dysthymia), Premenstrual Dysphoric Disorder, Substance/Medication-Induced Depressive Disorder, Post-Partum Depression, Depressive Disorder due to Another Medical Condition, Separation Anxiety Disorder, Selective Mutism, Specific Phobia, Social Anxiety Disorder (Social Phobia), Panic Disorder, Panic Attack, Agoraphobia, Generalized Anxiety Disorder, Substance-Medication-Induced Anxiety Disorder, Anxiety Disorder Due to Another Medical Condition, Somatic Symptom Disorder, Illness Anxiety Disorder (hypochondriac), Conversion Disorder (Functional Neurological Symptom Disorder), Factitious Disorder, Post-Traumatic Stress Disorder (PTSD), Adjustment Disorders, Acute Distress Disorder, Obsessive-Compulsive Disorder, Body Dysmorphic Disorder, Hoarding Disorder, Trichotillomania (Hair-Pulling) Disorder, Excoriation (Skin-Picking) Disorder, Substance/Medication-Induced Obsessive-Compulsive and Related Disorder, Obsessive-Compulsive and Related Disorder due to Another Medical Condition, Substance-Related Disorders, Alcohol-Related Disorders, Cannabis-Related Disorders, Hallucinogen-Related Disorders, Inhalant-Related Disorders, Cocaine-Related Disorders, Opioid-Related Disorders, Sedative-, Hypnotic-, or Anxiolytic-Related Disorders, Stimulant-Related Disorders, Tobacco-Related Disorders, Non-Substance-Related Disorders (Gambling or Gaming Disorder), Migraines, Cluster Headaches (including Chronic Cluster Headaches), Cyclical Vomiting, Tension-Type Headache, Dysphasia, Pica, Anorexia Nervosa, Bulimia Nervosa, Binge-Eating Disorder, Oppositional Defiant Disorder, Intermittent Explosive Disorder, Conduct Disorder, Antisocial Personality Disorder, Psychopathy, Pyromania, or Kleptomania.

In some embodiments, the patient is suffering from treatment-resistant depression. In some embodiments, a patient with treatment-resistant depression is staged using the Maudsley staging method for treatment resistance in depression. The following document describes this method and is incorporated by reference herein in its entirety: Fekadu et al. BMC Psychiatry (2018) 18:100.

### Methods for Treating

Provided herein are methods of treating a patient in need thereof, the method comprising administering to the patient a therapeutically-effective dose of psilocybin.

The methods described herein may be used to treat a variety of diseases, disorders, or conditions including particular psychiatric and neurological aspects of the diseases, disorders, or conditions.

In some embodiments, a method of treating a patient in need thereof comprises administering to the patient a therapeutically-effective dose of psilocybin wherein the patient suffers from Disruptive Mood Dysregulation Disorder, Major Depressive Disorder (MDD), Treatment-Resistant Depression, Persistent Depressive Disorder (Dysthymia), Premenstrual Dysphoric Disorder, Substance/Medication-Induced Depressive Disorder, Post-Partum Depression, Depressive Disorder due to Another Medical Condition, Separation Anxiety Disorder, Selective Mutism, Specific Phobia, Social Anxiety Disorder (Social Phobia), Panic Disorder, Panic Attack, Agoraphobia, Generalized Anxiety Disorder, Substance-Medication-Induced Anxiety Disorder, Anxiety Disorder Due to Another Medical Condition, Somatic Symptom Disorder, Illness Anxiety Disorder (hypochondriac), Conversion Disorder (Functional Neurological Symptom Disorder), Factitious Disorder, Post-Traumatic Stress Disorder (PTSD), Adjustment Disorders, Acute Distress Disorder, Obsessive-Compulsive Disorder, Body Dysmorphic Disorder, Hoarding Disorder, Trichotillomania (Hair-Pulling) Disorder, Excoriation (Skin-Picking) Disorder, Substance/Medication-Induced Obsessive-Compulsive and Related Disorder, Obsessive-Compulsive and Related Disorder due to Another Medical Condition, Substance-Related Disorders, Alcohol-Related Disorders, Cannabis-Related Disorders, Hallucinogen-Related Disorders, Inhalant-Related Disorders, Cocaine-Related Disorders, Opioid-Related Disorders, Sedative-, Hypnotic-, or Anxiolytic-Related Disorders, Stimulant-Related Disorders, Tobacco-Related Disorders, Non-Substance-Related Disorders (Gambling or Gaming Disorder), Migraines, Cluster Headaches (including Chronic Cluster Headaches), Cyclical Vomiting, Tension-Type Headache, Dysphasia, Pica, Anorexia Nervosa, Bulimia Nervosa, Binge-Eating Disorder, Oppositional Defiant Disorder, Intermittent Explosive Disorder, Conduct Disorder, Antisocial Personality Disorder, Psychopathy, Pyromania, or Kleptomania.

In some embodiments, the methods of treatment comprising administering psilocybin to a patient in need thereof further comprise pretreating the patient with magnesium before administration of the psilocybin. Sometimes, magnesium is administered daily for a least 1 day, at least 2 days, at least 3 days, at least 4 days, at least 5 days, at least 6 days, at least 1 week, at least 2 weeks, at least 3 weeks, at least 4 weeks, at least 5 weeks, or at least 6 weeks before administration of the psilocybin. In some embodiments, about 10 mg to about 500 mg of magnesium are administered to the patient per day. In some embodiments, about 30 mg, about 75 mg, about 80 mg, about 130 mg, about 240 mg, about 310 mg, about 320 mg, about 360 mg, about 410 mg, about 400 mg, or about 420 mg are administered to the patient per day. In some embodiments the magnesium is administered to the patient on the same day as the psilocybin. In some embodiments, the magnesium is administered to the patient immediately before, concurrently with, or immediately after administration of the psilocybin. In some embodiments, magnesium supplements are administered to the patient until the patient's blood level for magnesium is about 1.5 to about 2.5 mEq/L. In some embodiments, psilocybin is not administered to the patient if the patient's blood level of magnesium is less than about 1.5 to about 2.5 mEq/L.

In some embodiments, the methods comprise administering psilocybin to a subject in need thereof, wherein prior to administration of psilocybin, the subject was on a selective serotonin reuptake inhibitor (SSRI) therapy regimen. In some embodiments, the methods comprise ceasing SSRI therapy prior to administration of psilocybin and administering one or more benzodiazepines to a subject prior to administration of psilocybin. In some embodiments, SSRI therapy is ceased immediately without a titration period. In some embodiments, one or more benzodiazepines is administered during an SSRI maintenance period prior to administration of psilocybin, wherein administration of an SSRI is ceased immediately within 1 to 35 days of administration of the one or more benzodiazepines. In some embodiments, one or more benzodiazepines is administered during an SSRI maintenance period prior to administration of psilocybin, wherein administration of an SSRI is ceased during a titration period within 1 to 35 days of administration of the one or more benzodiazepines. In some embodiments, one or more benzodiazepines is administered during a SSRI titration period prior to administration of psilocybin, wherein during the SSRI titration period, the dose of SSRI is reduced from a maintenance period to cessation. In some embodiments, one or more benzodiazepines is administered to a subject prior to administration of psilocybin, wherein the subject has ceased SSRI administration prior to administration of the one or more benzodiazepines.

In some embodiments, the methods of treatment comprising administering psilocybin to a patient in need thereof disclosed herein further comprise providing psychological support to the patient during the administration and for at least an hour thereafter. In certain embodiments, psychological support is provident during administration and for at least 2 hours thereafter, at least 3 hours thereafter, at least 4 hours thereafter, at least 5 hours thereafter, at least 6 hours thereafter, at least 7 hours thereafter, at least 8 hours thereafter, at least 9 hours thereafter, at least 10 hours thereafter, at least 11 hours thereafter, at least 12 hours thereafter, at least 13 hours thereafter, at least 14 hours thereafter, or at least 15 hours thereafter. In particular embodiments, psychological support is provident during administration and for at least one to 15 hours thereafter, at least 2 to 15 hours thereafter, at least 3 to 15 hours thereafter, at least 4 to 15 hours thereafter, at least 5 to 15 hours thereafter, at least 2 to 14 hours thereafter, at least 3 to 14 hours thereafter, at least 4 to 14 hours thereafter, at least 5 to 14 hours thereafter, at least 2 to 12 hours thereafter, at least 3 to 12 hours thereafter, at least 4 to 12 hours thereafter, at least 5 to 12 hours thereafter, at least 2 to 10 hours thereafter, at least 3 to 10 hours thereafter, at least 4 to 10 hours thereafter, at least 5 to 10 hours thereafter, at least 2 to 8 hours thereafter, at least 3 to 8 hours thereafter, at least 4 to 8 hours thereafter, at least 5 to 8 hours thereafter, at least 2 to 6 hours thereafter, at least 3 to 6 hours thereafter, at least 4 to 6 hours thereafter, at least 5 to 6 hours thereafter, or at least 2 to 4 hours thereafter

In certain embodiments, the psychological support is psychotherapy support.

In particular embodiments, the psychotherapy is a transdiagnostic therapy. In further embodiments, the transdiagnostic therapy is a Method of Levels (MOL) therapy. In still further embodiments, the Method of Levels (MOL) therapy comprises Self-directed Enquiry and Experiential Processing.

MOL uses brief, but detailed, curious questioning to help patients shift and sustain their attention towards different levels of cognition and emotions (Carey, 2006; Carey, Mansell & Tai, 2015). The emphasis within MOL is on identifying and working with a patient's underlying distress as opposed to just their symptoms.

The MOL technique uses, two key principles:
Self-directed enquiry—directing attention to internal states. Participants are encouraged to be curious about experiences in the present moment, including foreground and background thoughts, emotions, and physical sensations. During the preparation and integration stages, this enquiry might mean asking specific and detailed questions to help direct attention to internal states. However, during the period of drug action, enquiry might simply mean an attitude of openness to inner experiences.
Experiential processing—sustained focus on the experience. Experiential processing refers to a participant's ability to maintain full attention on the experiences that come into awareness through self-directed enquiry. This includes a willingness and ability to be with and/or move ‘in and through’ even uncomfortable or challenging thoughts, feelings, sensations, or emotions, until discomfort is diminished or resolved.

In some embodiments, at least one symptom of a disease, disorder, or condition described herein is alleviated within 24 hours of administering psilocybin. In some embodiments, at least one symptom of the disease, disorder, or condition is alleviated within 1 week of the administering. In some embodiments, at least one symptom of the disease, disorder, or condition is alleviated within 1 month of the administering. In some embodiments, at least one symptom of the disease, disorder, or condition is alleviated within 6 months of the administering. In some embodiments, at least one symptom of the disease, disorder, or condition is alleviated within 12 months of the administering.

In some embodiments, at least one symptom of the disease, disorder, or condition is alleviated for a period of at least 1 month after administering psilocybin. In some embodiments, at least one symptom of the disease, disorder, or condition is alleviated for a period of at least 3 months after the administering. In some embodiments, at least one symptom of the disease, disorder, or condition is alleviated for a period of at least 6 months after the administering. In some embodiments, at least one symptom of the disease, disorder, or condition is alleviated for a period of at least 12 months after the administering.

In some embodiments, the methods of the disclosure provide for the alleviation of at least one side effect of SSRI washout within about 1 day after benzodiazepine administration. In some embodiments, the methods of the disclosure provide for the alleviation of at least one side effect of SSRI washout within about 1 week after benzodiazepine administration. In some embodiments, the methods of the disclosure provide for the alleviation of at least one side effect of SSRI washout within about 1 month after benzodiazepine administration. Non-limiting examples of side effects of SSRI washout include headache, asthenia, flu syndrome, fever, vasodilation, nausea, diarrhea, anorexia, dry mouth, dyspepsia, constipation, flatulence, vomiting, weight loss, insomnia, nervousness, anxiety, somnolence, dizziness, tremor, decreased libido, abnormal thinking, sweating, rash, pruritus, abnormal vision, dyspepsia, abdominal pain, fatigue, arthralgia, myalgia, somnolence, agitation, dysmenorrhea, decreased libido, yawning, upper respiratory tract infection, rhinitis, sinusitis, ejaculation disorder, ejaculatory delay, impotence, dysphoric mood, irritability, agitation, dizziness, sensory disturbances (e.g., paresthesias such as electric shock sensations), confusion, lethargy, emotional lability, and hypomania.

In some embodiments, alleviation of at least one side effect of SSRI is measured using a clinical rating scale, wherein the scale is selected from the group consisting of a Quick Inventory of Depressive Symptomatology (QIDS)-16 scale, a QIDS-16 daily scale, a Hamilton Depression Rating scale, a Beck Depression Inventory scale, a Montgomery-Asberg Depression Rating Scale, a Clinical Global Impression Scale, a Zung Self-Rating Depression Scale, a Raskin Depression Rating Scale, a Young Mania Rating Scale, a Spielberger's Trait and Anxiety Inventory, a Generalized Anxiety Disorder 7-Item Scale, a Warwick-Edinburgh Mental Wellbeing Scale, a Flourishing Scale, a Snaith Hamilton Anhedonia Pleasure Scale, a Life Orientation Test, a Meaning in Life Questionnaire, a Brief Resilience Scale, a Dysfunctional Attitudes Scale, a 44-item Big Five Inventory, a Peters 21-item Delusional Inventory, an Examination of Anomalous Self-Experience, a Ruminative Responses Scale, a White Bear Suppression Inventory, a Barrett Impulsivity Scale, a Brief Experiential Avoidance Questionnaire, a Modified Tellegen Absorption Questionnaire, a Scale to Assess Therapeutic Relationship, a Credibility/Expectancy Questionnaire, a Connectedness to Nature Scale, a Political Perspective Questionnaire, a Social Connectedness Scale, a Bech-Rafaelsen Mania Rating Scale, a Revised Santa Clara brief compassion scale, a Gratitude Questionnaire, a Short Suggestibility Scale, a Rosenberg Self-Esteem Scale, a Universality Subscale of the Spiritual Transcendence Scale, an Oxford Questionnaire on the Emotional Side-effects of Antidepressants, a Lauks Emotional Intensity Scale, a Sexual Dysfunction Questionnaire, a Brief Index of Sexual Functioning for Women, a Sexual Perceptions Questionnaire, a Barnes Akathisia Rating Scale, a Work Productivity and Activity Impairment Questionnaire, a Work and Social Adjustment Scale, a Connectedness Questionnaire, a Standard Assessment of Personality, a Positive and Negative Syndrome Scale, a Mastery Insight Scale, a Self-Reflection and Insight Scale, a Psychological Insight Scale, a Metaphysical Beliefs Questionnaire, a Spiritual Bypassing Scale, an Adverse Childhood Experience Questionnaire, a Therapeutic Music Experience Questionnaire, a Setting Questionnaire, an Absorption in Music Scale, a Psychedelic Predictor Scale, a Surrender Scale, a EuroQOL-5 Dimension-3 Level Scale, a Columbia-Suicide Severity Rating Scale, a Suicidal Ideation Attributes Scale, or any combinations thereof.

In some embodiments, no other treatment is administered to the patient to treat the disease, disorder, or condition before administration of the psilocybin. In some embodiments, no other treatment is administered to the patient to treat the disease, disorder, or condition after administration of the psilocybin.

### EXAMPLES

The following examples, which are included herein for illustration purposes only, are not intended to be limiting.

### Example 1: Treating a Patient with High Dose Psilocybin

Initially, a patient is counseled as to the expected effects of psilocybin by a professional who is trained to administer psilocybin therapy. One or more tablets or capsules comprising psilocybin are administered to the patient, in an environment where the patient is made to feel safe and comfortable. The total dose of psilocybin administered to the patient is between about 1 mg to about 25 mg.

The patient is supervised by the professional during administration of the psilocybin, and for a period of time thereafter (e.g., from about 4 hours to about 12 hours) until the psychoactive effects of the psilocybin have worn off. Optionally, the patient may receive psychological support during administration of the psilocybin, and for a period of time thereafter (e.g., from about 4 hours to about 12 hours).

### Example 2: Effect of Reducing the SSRI Washout Period in Patients Prior to Psilocybin Therapy

An in vivo rat model is used to examine whether administration of a benzodiazepine will enhance the effects of psilocybin in mice. Rats are randomized into experimental groups (n=6 for each group). Treatments are administered in accordance with Table 9 below by scientific staff blinded to treatment groups.

TABLE 9

| DAY | Group 1 | Group 2 | Group 3 | Group 4 | Group 5 | Group 6 | Group 7 |
|---|---|---|---|---|---|---|---|
| 1-14 | Vehicle | Vehicle | Chronic | Vehicle | Chronic | Vehicle | Chronic |
| | (Saline) | (Saline) | SSRI regimen | (Saline) | SSRI regimen | (Saline) | SSRI regimen |
| | | | (Paroxetine) | | (Paroxetine) | | (Paroxetine) |
| 5-15 | Vehicle | Vehicle | Vehicle | Benzodiazepine | Benzodiazepine | Benzodiazepine | Benzodiazepine |
| | (Saline) | (Saline) | (Saline) | (5 mg/kg | (5 mg/kg | (10 mg/kg | (10 mg/kg |
| | | | | Clonazepam) | Clonazepam) | Clonazepam) | Clonazepam) |
| 16 | Vehicle | Psilocybin | Psilocybin | Psilocybin | Psilocybin | Psilocybin | Psilocybin |
| | (Saline) | (3 mg/kg) | (3 mg/kg) | (3 mg/kg) | (3 mg/kg) | (3 mg/kg) | (3 mg/kg) |

The efficacy of psilocybin is evaluated by a head twitch assay. The head twitch response (HTR) is used as a proxy for psychedelic effects of a compound. The HTR consists of rapid and violent head twitching. Enhancement of the head twitch response represents an improved response to psilocybin. (Halberstadt et al., 2020). 5-HT2A receptor expression is also be evaluated via quantitative PCR, surface binding of radioligands, immunohistochemistry, and flow cytometry.

### Example 3: Effects of Acute Benzodiazepine Pretreatment on the Head Twitch Response to Psilocybin

Animals were dosed as detailed by Table 10 below and were placed in a clean cage. The number of head twitches and wet dog shakes were then counted by a scorer for 30 minutes after dosing. The timings of head twitches and wet dog shakes were recorded by a computer program. The experiment was carried out over three days. Scorers were blinded to treatment group.

TABLE 10

| Group | Treatment | Psilocybin challenge (sc) | n |
|---|---|---|---|
| A | Diazepam i.p 4 mg/kg | 0.3 mg/kg, 1 hr post Diazepam | 12 |
| | single dose | | |
| B | Saline i.p single dose | 0.3 mg/kg, 1 hr post saline | 12 |
| C | Diazepam i.p 4 mg/kg | 1 mg/kg, 1 hr post Diazepam | 12 |
| | single dose | | |
| D | Saline i.p single dose | 1 mg/kg, 1 hr post saline | 12 |
| E | Diazepam i.p 4 mg/kg | 3 mg/kg, 1 hr post Diazepam | 12 |
| | single dose | | |
| F | Saline i.p single dose | 3 mg/kg, 1 hr post saline | 12 |

### Data Examination

There were no wet dog shakes recorded, so head twitches were organized into 5-minute and 15-minute time periods for each animal. Shapiro-Wilk tests indicated that for most of the 5-minute and 15-minute time windows, square root transformed head twitch counts were normally distributed.

Ethovision data was organized into 5-minute and 15-minute time periods and 0-30 minutes for each animal. Ethovision scores were log transformed, however Shapiro-Wilk tests were significant for many of the 5-minute and 15-minute time windows. Therefore, robust regression was used to analyze the Ethovision data.

### Statistical Methods

Number of head twitches over each 5-minute period and each 15-minute period and the total for 0-30 minutes were square-root transformed and analyzed by three way ANOVA with treatment, day, and scorer as factors. Ethovision data was analyzed by robust regression using M estimation, Huber weighting, using the default parameter C=1.345. The model used treatment, day, and scorer as factors.

Each diazepam group was compared to the vehicle group which received the same dose of Psilocybin by multiple t-test. P<0.05 was the level accepted for statistical difference. The chance of a false positive is 5% for each dose for each time. All tests were carried out as two-sided tests.

TABLE 11
Head Twitches over 5-minute intervals

| Time | | | | | | |
|---|---|---|---|---|---|---|
| period | Treatment | n | Mean | SEM | % | p |
| 0 to | Diazepam 4 mg/kg ip/Psilocybin 0.3 mg/kg sc | 12 | 0.60 | 0.35 | 32.5 | 0.063 |
| 5 mins | Saline ip/Psilocybin 0.3 mg/kg sc | 12 | 1.84 | 0.35 | | |
| | Diazepam 4 mg/kg ip/Psilocybin 1 mg/kg sc | 12 | 0.71 | 0.46 | 29.3 | 0.022* |
| | Saline ip/Psilocybin 1 mg/kg sc | 12 | 2.42 | 0.49 | | |
| | Diazepam 4 mg/kg ip/Psilocybin 3 mg/kg sc | 12 | 7.25 | 1.05 | 135.8 | 0.212 |
| | Saline ip/Psilocybin 3 mg/kg sc | 12 | 5.34 | 1.12 | | |
| 5 to | Diazepam 4 mg/kg ip/Psilocybin 0.3 mg/kg sc | 12 | 1.49 | 0.52 | 90.7 | 0.820 |
| 10 mins | Saline ip/Psilocybin 0.3 mg/kg sc | 12 | 1.64 | 0.36 | | |
| | Diazepam 4 mg/kg ip/Psilocybin 1 mg/kg sc | 12 | 3.38 | 0.80 | 243.1 | 0.015* |
| | Saline ip/Psilocybin 1 mg/kg sc | 12 | 1.39 | 0.39 | | |
| | Diazepam 4 mg/kg ip/Psilocybin 3 mg/kg sc | 12 | 5.51 | 0.73 | 118.1 | 0.481 |
| | Saline ip/Psilocybin 3 mg/kg sc | 12 | 4.67 | 0.79 | | |
| 10 to | Diazepam 4 mg/kg ip/Psilocybin 0.3 mg/kg sc | 12 | 2.06 | 0.61 | 152.0 | 0.365 |
| 15 mins | Saline ip/Psilocybin 0.3 mg/kg sc | 12 | 1.36 | 0.33 | | |
| | Diazepam 4 mg/kg ip/Psilocybin 1 mg/kg sc | 12 | 3.01 | 0.85 | 208.7 | 0.072 |
| | Saline ip/Psilocybin 1 mg/kg sc | 12 | 1.44 | 0.48 | | |
| | Diazepam 4 mg/kg ip/Psilocybin 3 mg/kg sc | 12 | 1.82 | 0.48 | 87.7 | 0.756 |
| | Saline ip/Psilocybin 3 mg/kg sc | 12 | 2.07 | 0.62 | | |
| 15 to | Diazepam 4 mg/kg ip/Psilocybin 0.3 mg/kg sc | 12 | 1.98 | 0.59 | 212.6 | 0.109 |
| 20 mins | Saline ip/Psilocybin 0.3 mg/kg sc | 12 | 0.93 | 0.38 | | |
| | Diazepam 4 mg/kg ip/Psilocybin 1 mg/kg sc | 12 | 1.16 | 0.47 | 85.7 | 0.747 |
| | Saline ip/Psilocybin 1 mg/kg sc | 12 | 1.35 | 0.38 | | |
| | Diazepam 4 mg/kg ip/Psilocybin 3 mg/kg sc | 12 | 0.31 | 0.15 | 29.3 | 0.081 |
| | Saline ip/Psilocybin 3 mg/kg sc | 12 | 1.07 | 0.35 | | |
| 20 to | Diazepam 4 mg/kg ip/Psilocybin 0.3 mg/kg sc | 12 | 1.14 | 0.57 | 290.3 | 0.084 |
| 25 mins | Saline ip/Psilocybin 0.3 mg/kg sc | 12 | 0.39 | 0.19 | | |
| | Diazepam 4 mg/kg ip/Psilocybin 1 mg/kg sc | 12 | 0.12 | 0.10 | 28.0 | 0.217 |
| | Saline ip/Psilocybin 1 mg/kg sc | 12 | 0.43 | 0.16 | | |
| | Diazepam 4 mg/kg ip/Psilocybin 3 mg/kg sc | 12 | 0.02 | 0.03 | 2.6 | 0.008** |
| | Saline ip/Psilocybin 3 mg/kg sc | 12 | 0.66 | 0.27 | | |
| 25 to | Diazepam 4 mg/kg ip/Psilocybin 0.3 mg/kg sc | 12 | 0.49 | 0.30 | 83.8 | 0.770 |
| 30 mins | Saline ip/Psilocybin 0.3 mg/kg sc | 12 | 0.59 | 0.26 | | |
| | Diazepam 4 mg/kg ip/Psilocybin 1 mg/kg sc | 12 | 0.00 | −0.00 | 0.0 | 0.083 |
| | Saline ip/Psilocybin 1 mg/kg sc | 12 | 0.15 | 0.11 | | |
| | Diazepam 4 mg/kg ip/Psilocybin 3 mg/kg sc | 12 | 0.00 | 0.00 | 0.0 | 0.011* |
| | Saline ip/Psilocybin 3 mg/kg sc | 12 | 0.34 | 0.21 | | |

Means are back transformed and adjusted. SEMs calculated from the residuals of the statistical model. Comparison of each diazepam group to the vehicle group which received the same dose of Psilocybin was by the multiple t test.
*p < 0.05,
**p < 0.01.

TABLE 12
Head Twitches over 15-minute intervals

| Time | | | | | | |
|---|---|---|---|---|---|---|
| period | Treatment (po) | n | Mean | SEM | % | p |
| 0 to | Diazepam 4 mg/kg ip/Psilocybin 0.3 mg/kg sc | 12 | 4.81 | 1.27 | 86.2 | 0.612 |
| 15 mins | Saline ip/Psilocybin 0.3 mg/kg sc | 12 | 5.57 | 0.47 | | |
| | Diazepam 4 mg/kg ip/Psilocybin 1 mg/kg sc | 12 | 8.62 | 1.27 | 136.5 | 0.197 |
| | Saline ip/Psilocybin 1 mg/kg sc | 12 | 6.32 | 0.91 | | |
| | Diazepam 4 mg/kg ip/Psilocybin 3 mg/kg sc | 12 | 15.32 | 1.43 | 123.6 | 0.230 |
| | Saline ip/Psilocybin 3 mg/kg sc | 12 | 12.40 | 2.16 | | |
| 15 to | Diazepam 4 mg/kg ip/Psilocybin 0.3 mg/kg sc | 12 | 4.14 | 1.37 | 161.0 | 0.185 |
| 30 mins | Saline ip/Psilocybin 0.3 mg/kg sc | 12 | 2.57 | 0.46 | | |
| | Diazepam 4 mg/kg ip/Psilocybin 1 mg/kg sc | 12 | 1.54 | 0.51 | 64.5 | 0.339 |
| | Saline ip/Psilocybin 1 mg/kg sc | 12 | 2.39 | 0.43 | | |
| | Diazepam 4 mg/kg ip/Psilocybin 3 mg/kg sc | 12 | 0.37 | 0.21 | 15.0 | 0.003** |
| | Saline ip/Psilocybin 3 mg/kg sc | 12 | 2.50 | 0.77 | | |

Means are back transformed and adjusted. SEMs calculated from the residuals of the statistical model. Comparison of each diazepam group to the vehicle group which received the same dose of Psilocybin was by the multiple t test.
**p < 0.001.

TABLE 13
Head Twitch totals

| Time | | | | | | |
|---|---|---|---|---|---|---|
| period | Treatment (po) | n | Mean | SEM | % | p |
| 0 to | Diazepam 4 mg/kg ip/Psilocybin 0.3 mg/kg sc | 12 | 9.41 | 2.59 | 112.2 | 0.677 |
| 30 mins | Saline ip/Psilocybin 0.3 mg/kg sc | 12 | 8.38 | 0.72 | | |
| | Diazepam 4 mg/kg ip/Psilocybin 1 mg/kg sc | 12 | 10.48 | 1.61 | 116.6 | 0.554 |
| | Saline ip/Psilocybin 1 mg/kg sc | 12 | 8.98 | 1.26 | | |
| | Diazepam 4 mg/kg ip/Psilocybin 3 mg/kg sc | 12 | 16.04 | 1.61 | 102.4 | 0.906 |
| | Saline ip/Psilocybin 3 mg/kg sc | 12 | 15.66 | 2.80 | | |

Means are back transformed and adjusted. SEMs calculated from the residuals of the statistical model. Comparison of each diazepam group to the vehicle group which received the same dose of Psilocybin was by the multiple t test.

TABLE 14
Ethovision data over 5-minute intervals

| Time | | | | | | |
|---|---|---|---|---|---|---|
| period | Treatment (po) | n | Mean | SEM | % | p |
| 0 to | Diazepam 4 mg/kg ip/Psilocybin 0.3 mg/kg sc | 12 | 494.04 | 42.66 | 61.6 | <0.001*** |
| 5 mins | Saline ip/Psilocybin 0.3 mg/kg sc | 12 | 801.48 | 33.37 | | |
| | Diazepam 4 mg/kg ip/Psilocybin 1 mg/kg sc | 12 | 381.84 | 70.01 | 46.2 | <0.001*** |
| | Saline ip/Psilocybin 1 mg/kg sc | 12 | 826.56 | 50.26 | | |
| | Diazepam 4 mg/kg ip/Psilocybin 3 mg/kg sc | 12 | 543.10 | 44.88 | 69.2 | 0.003** |
| | Saline ip/Psilocybin 3 mg/kg sc | 12 | 785.38 | 57.07 | | |
| 5 to | Diazepam 4 mg/kg ip/Psilocybin 0.3 mg/kg sc | 12 | 481.50 | 42.73 | 68.3 | 0.010* |
| 10 mins | Saline ip/Psilocybin 0.3 mg/kg sc | 12 | 704.55 | 41.46 | | |
| | Diazepam 4 mg/kg ip/Psilocybin 1 mg/kg sc | 12 | 419.16 | 55.29 | 62.9 | 0.002** |
| | Saline ip/Psilocybin 1 mg/kg sc | 12 | 665.97 | 31.19 | | |
| | Diazepam 4 mg/kg ip/Psilocybin 3 mg/kg sc | 12 | 301.09 | 34.61 | 58.4 | <0.001*** |
| | Saline ip/Psilocybin 3 mg/kg sc | 12 | 516.00 | 52.25 | | |
| 10 to | Diazepam 4 mg/kg ip/Psilocybin 0.3 mg/kg sc | 12 | 494.04 | 58.78 | 77.0 | 0.072 |
| 15 mins | Saline ip/Psilocybin 0.3 mg/kg sc | 12 | 641.76 | 35.04 | | |
| | Diazepam 4 mg/kg ip/Psilocybin 1 mg/kg sc | 12 | 364.32 | 70.54 | 70.2 | 0.014* |
| | Saline ip/Psilocybin 1 mg/kg sc | 12 | 519.27 | 26.25 | | |
| | Diazepam 4 mg/kg ip/Psilocybin 3 mg/kg sc | 12 | 137.34 | 21.14 | 38.9 | <0.001*** |
| | Saline ip/Psilocybin 3 mg/kg sc | 12 | 353.03 | 44.07 | | |
| 15 to | Diazepam 4 mg/kg ip/Psilocybin 0.3 mg/kg sc | 12 | 390.32 | 59.38 | 75.4 | 0.092 |
| 20 mins | Saline ip/Psilocybin 0.3 mg/kg sc | 12 | 517.51 | 44.37 | | |
| | Diazepam 4 mg/kg ip/Psilocybin 1 mg/kg sc | 12 | 175.77 | 29.58 | 40.9 | <0.001*** |
| | Saline ip/Psilocybin 1 mg/kg sc | 12 | 430.23 | 30.24 | | |
| | Diazepam 4 mg/kg ip/Psilocybin 3 mg/kg sc | 12 | 61.74 | 6.09 | 22.8 | <0.001*** |
| | Saline ip/Psilocybin 3 mg/kg sc | 12 | 271.26 | 25.85 | | |
| 20 to | Diazepam 4 mg/kg ip/Psilocybin 0.3 mg/kg sc | 12 | 236.11 | 48.57 | 45.4 | <0.001*** |
| 25 mins | Saline ip/Psilocybin 0.3 mg/kg sc | 12 | 519.96 | 50.23 | | |
| | Diazepam 4 mg/kg ip/Psilocybin 1 mg/kg sc | 12 | 72.99 | 13.57 | 16.4 | <0.001*** |
| | Saline ip/Psilocybin 1 mg/kg sc | 12 | 444.11 | 28.55 | | |
| | Diazepam 4 mg/kg ip/Psilocybin 3 mg/kg sc | 12 | 45.97 | 4.55 | 22.0 | <0.001*** |
| | Saline ip/Psilocybin 3 mg/kg sc | 12 | 208.90 | 25.28 | | |
| 25 to | Diazepam 4 mg/kg ip/Psilocybin 0.3 mg/kg sc | 12 | 122.72 | 41.38 | 22.3 | <0.001*** |
| 30 mins | Saline ip/Psilocybin 0.3 mg/kg sc | 12 | 551.06 | 43.96 | | |
| | Diazepam 4 mg/kg ip/Psilocybin 1 mg/kg sc | 12 | 84.88 | 16.98 | 19.7 | <0.001*** |
| | Saline ip/Psilocybin 1 mg/kg sc | 12 | 430.12 | 64.17 | | |
| | Diazepam 4 mg/kg ip/Psilocybin 3 mg/kg sc | 12 | 59.37 | 17.37 | 27.9 | <0.001*** |
| | Saline ip/Psilocybin 3 mg/kg sc | 12 | 213.04 | 28.30 | | |

Means are back transformed and adjusted. SEMs calculated from the residuals of the statistical model. Comparison of each diazepam group to the vehicle group which received the same dose of Psilocybin was by the multiple t test.
*p < 0.05,
**p < 0.01,
***p < 0.001.

TABLE 15
Ethovision data over 15-minute intervals

| Time | | | | | | |
|---|---|---|---|---|---|---|
| period | Treatment (po) | n | Mean | SEM | % | p |
| 0 to | Diazepam 4 mg/kg ip/Psilocybin 0.3 mg/kg sc | 12 | 1507.36 | 102.73 | 70.1 | <0.001*** |
| 15 mins | Saline ip/Psilocybin 0.3 mg/kg sc | 12 | 2149.79 | 90.95 | | |
| | Diazepam 4 mg/kg ip/Psilocybin 1 mg/kg sc | 12 | 1162.28 | 176.16 | 57.4 | <0.001*** |
| | Saline ip/Psilocybin 1 mg/kg sc | 12 | 2025.10 | 74.96 | | |
| | Diazepam 4 mg/kg ip/Psilocybin 3 mg/kg sc | 12 | 1030.51 | 74.81 | 61.8 | <0.001*** |
| | Saline ip/Psilocybin 3 mg/kg sc | 12 | 1667.64 | 129.02 | | |
| 15 to | Diazepam 4 mg/kg ip/Psilocybin 0.3 mg/kg sc | 12 | 855.00 | 144.89 | 52.4 | <0.001*** |
| 30 mins | Saline ip/Psilocybin 0.3 mg/kg sc | 12 | 1630.42 | 118.66 | | |
| | Diazepam 4 mg/kg ip/Psilocybin 1 mg/kg sc | 12 | 358.67 | 50.81 | 26.8 | <0.001*** |
| | Saline ip/Psilocybin 1 mg/kg sc | 12 | 1337.33 | 79.34 | | |
| | Diazepam 4 mg/kg ip/Psilocybin 3 mg/kg sc | 12 | 178.70 | 29.94 | 24.8 | <0.001*** |
| | Saline ip/Psilocybin 3 mg/kg sc | 12 | 719.96 | 74.34 | | |

Means are back transformed and adjusted. SEMs calculated from the residuals of the statistical model. Comparison of each diazepam group to the vehicle group which received the same dose of Psilocybin was by the multiple t test.
***p < 0.001.

TABLE 16
Ethovision totals

| Time | | | | | | |
|---|---|---|---|---|---|---|
| period | Treatment (po) | n | Mean | SEM | % | p |
| 0 to | Diazepam 4 mg/kg ip/Psilocybin 0.3 mg/kg sc | 12 | 2460.76 | 230.67 | 65.1 | <0.001*** |
| 30 mins | Saline ip/Psilocybin 0.3 mg/kg sc | 12 | 3782.48 | 168.05 | | |
| | Diazepam 4 mg/kg ip/Psilocybin 1 mg/kg sc | 12 | 1706.43 | 231.41 | 50.7 | <0.001*** |
| | Saline ip/Psilocybin 1 mg/kg sc | 12 | 3368.87 | 123.05 | | |
| | Diazepam 4 mg/kg ip/Psilocybin 3 mg/kg sc | 12 | 1298.59 | 83.00 | 54.0 | <0.001*** |
| | Saline ip/Psilocybin 3 mg/kg sc | 12 | 2403.79 | 181.48 | | |

Means are back transformed and adjusted. SEMs calculated from the residuals of the statistical model. Comparison of each diazepam group to the vehicle group which received the same dose of Psilocybin was by the multiple t test.
***p < 0.001.

TABLE 17
Extreme values in head twitch data, using criterion z <− 3 or z > 3

| Mouse | Treatment | Measurement | Value | z |
|---|---|---|---|---|
| 8 | A | 20 to 25 mins | 8.00 | 3.32 |
| 4 | F | 0 to 15 mins | 0.00 | −3.51 |
| 4 | F | 0 to 30 mins | 0.00 | −3.29 |

z is the ‘studentised residual’ from the statistical model - for normally distributed data it is less than −3 or greater than 3 for about 1 in 400 observations.

TABLE 18
Extreme values in Ethovision data, using criterion z <− 3 or z > 3

| Mouse | Treatment | Measurement | Value | z |
|---|---|---|---|---|
| 11 | C | 0 to 5 mins | 1050.69 | 4.91 |
| 25 | C | 0 to 5 mins | 183.08 | −3.82 |
| 27 | C | 0 to 5 mins | 1412.55 | 4.07 |
| 5 | F | 5 to 10 mins | 1526.01 | 3.12 |
| 39 | A | 10 to 15 mins | 135.99 | −3.61 |
| 51 | C | 10 to 15 mins | 66.49 | −4.92 |
| 71 | C | 10 to 15 mins | 72.32 | −4.49 |
| 57 | E | 10 to 15 mins | 45.85 | −3.88 |
| 5 | F | 10 to 15 mins | 1204.88 | 3.26 |
| 39 | A | 15 to 20 mins | 101.23 | −3.47 |
| 38 | A | 20 to 25 mins | 1063.00 | 3.20 |
| 68 | A | 20 to 25 mins | 62.49 | −3.21 |
| 49 | C | 20 to 25 mins | 598.30 | 4.77 |
| 8 | A | 25 to 30 mins | 706.91 | 3.43 |
| 24 | A | 25 to 30 mins | 560.30 | 3.88 |
| 38 | A | 25 to 30 mins | 967.19 | 3.58 |
| 39 | A | 25 to 30 mins | 27.76 | −3.28 |
| 16 | E | 25 to 30 mins | 639.81 | 4.69 |
| 44 | E | 25 to 30 mins | 549.19 | 4.19 |
| 11 | C | 0 to 15 mins | 2037.86 | 4.06 |
| 25 | C | 0 to 15 mins | 637.36 | −4.77 |
| 27 | C | 0 to 15 mins | 2736.59 | 4.18 |
| 71 | C | 0 to 15 mins | 416.23 | −4.95 |
| 5 | F | 0 to 15 mins | 4721.56 | 4.91 |
| 39 | A | 15 to 30 mins | 275.40 | −3.96 |
| 49 | C | 15 to 30 mins | 1634.77 | 4.18 |
| 16 | E | 15 to 30 mins | 794.30 | 4.93 |
| 44 | E | 15 to 30 mins | 625.29 | 3.89 |
| 39 | A | 0 to 30 mins | 1110.68 | −3.27 |
| 25 | C | 0 to 30 mins | 1001.74 | −3.44 |
| 71 | C | 0 to 30 mins | 625.86 | −3.77 |
| 5 | F | 0 to 30 mins | 5784.05 | 3.31 |

z is the ‘studentised residual’ from the statistical model - for normally distributed data it is less than −3 or greater than 3 for about 1 in 400 observations.

TABLE 19

| | Means | | | | | | | | |
|---|---|---|---|---|---|---|---|---|---|
| | 0-5 | 5-10 | 10-15 | 15-20 | 20-25 | 25-30 | 0-15 | 15-30 | 0-30 |
| | mins | mins | mins | mins | mins | mins | mins | mins | mins |
| Diazepam/ | 0.6 | 1.49 | 2.06 | 1.98 | 1.14 | 0.49 | 4.81 | 4.14 | 9.41 |
| 0.3 mg/kg | | | | | | | | | |
| psilocybin | | | | | | | | | |
| (A) | | | | | | | | | |
| Saline/ | 1.84 | 1.64 | 1.36 | 0.93 | 0.39 | 0.59 | 5.57 | 2.57 | 8.38 |
| 0.3 mg/kg | | | | | | | | | |
| psilocybin | | | | | | | | | |
| (B) | | | | | | | | | |
| Diazepam/ | 0.71 | 3.38 | 3.01 | 1.16 | 0.12 | 0 | 8.62 | 1.54 | 10.48 |
| 1 mg/kg | | | | | | | | | |
| psilocybin | | | | | | | | | |
| (C) | | | | | | | | | |
| Saline/ | 2.42 | 1.39 | 1.44 | 1.35 | 0.43 | 0.15 | 6.32 | 2.39 | 8.98 |
| 1 mg/kg | | | | | | | | | |
| psilocybin | | | | | | | | | |
| (D) | | | | | | | | | |
| Diazepam/ | 7.25 | 5.51 | 1.82 | 0.31 | 0.02 | 0 | 15.32 | 0.37 | 16.04 |
| 3 mg/kg | | | | | | | | | |
| psilocybin | | | | | | | | | |
| (E) | | | | | | | | | |
| Saline/ | 5.34 | 4.67 | 2.07 | 1.07 | 0.66 | 0.34 | 12.4 | 2.5 | 15.66 |
| 3 mg/kg | | | | | | | | | |
| psilocybin | | | | | | | | | |
| (F) | | | | | | | | | |
| SEMS | | | | | | | | | |
| A | 0.35 | 0.52 | 0.61 | 0.59 | 0.57 | 0.3 | 1.27 | 1.37 | 2.59 |
| B | 0.35 | 0.36 | 0.33 | 0.38 | 0.19 | 0.26 | 0.47 | 0.46 | 0.72 |
| C | 0.46 | 0.8 | 0.85 | 0.47 | 0.1 | 0 | 1.27 | 0.51 | 1.61 |
| D | 0.49 | 0.39 | 0.48 | 0.38 | 0.16 | 0.11 | 0.91 | 0.43 | 1.26 |
| E | 1.05 | 0.73 | 0.48 | 0.15 | 0.03 | 0 | 1.43 | 0.21 | 1.61 |
| F | 1.12 | 0.79 | 0.62 | 0.35 | 0.27 | 0.21 | 2.16 | 0.77 | 2.8 |
| p-vals | | | | | | | | | |
| A | 0.063 | 0.82 | 0.365 | 0.109 | 0.084 | 0.77 | 0.612 | 0.185 | 0.677 |
| C | 0.022* | 0.015* | 0.072 | 0.747 | 0.217 | 0.083 | 0.197 | 0.339 | 0.554 |
| E | 0.212 | 0.481 | 0.756 | 0.081 | 0.008** | 0.011* | 0.23 | 0.003** | 0.906 |

### Example 4. Modulation of Psilocybin Head Twitch Response with Chronic Diazepam Pre-Treatment

Sixty-two (60 plus 2 spares;) male C57BL/6J mice (20-25 g upon arrival) were purchased from Charles River UK. Animals were group housed (in 3 s and a pair) in polypropylene cages. Mice were maintained on a normal phase 12 h light-dark cycle (lights on: 07:00-19:00 h) with free access to standard rodent maintenance diet (standard pelleted diet Envigo 2018) and filtered tap water. The holding room was maintained at a temperature of 21±4° C. and relative humidity was 55±15% with prolonged periods below 40% RH or above 70% RH avoided as detailed in the UK Code of Practice. As a refinement, each cage contained sawdust, sizzle nest, a red house, Perspex tunnel, plastic chew stick and a nestlet so that the mice can make nests and facilitate warmth. Mice were weighed upon arrival and provided with wet mash overnight. The following morning, mice were weighed, and the mash removed. Mice will be weighed again on Monday. Any mice exhibiting weight loss were be given wet mash.

### Experimental Procedures

Mice were weighed and allocated into 6 treatment groups by a statistician (as per Table 20 below). Animals were administered diazepam (1.25 mg/kg, i.p) or saline (i.p) for 14 days and then received a psilocybin challenge followed by a head twitch response assessment or had brain tissue removed for binding analysis, according to the table below. For groups A-D animals were placed individually in clean cages with a light dusting of sawdust immediately following psilocybin administration (0.3 mg/kg, s.c) for head twitch response assessment. Head twitches were counted by a trained observer over a 30 minute period.

For groups A and B this occurred 2 h following the final diazepam dose, and for C and D the identical procedure occurred but 24 h post diazepam. Statistical significance was tested using two-way analysis of variance with treatment and observer as cofactors (Table 21 below).

For analysis of 5HT2A binding, homogenates were prepared from frontal cortices sampled from groups E and F 24 h following the final diazepam administration (See Table 22 and 23 below). To prepare the homogenate, frontal cortices from each animal were homogenized individually in 40 volumes (w/v) of ice cold assay buffer using a tight-fitting glass/Teflon homogenizer and centrifuged at 39,500×g for 10 min. The supernatant was discarded, and the pellet re-suspended in 40 volumes (w/v) of assay buffer and the tissue washed by centrifugation two more times. The final pellet was re-suspended in ice-cold assay buffer to 6.25 mg weight wet of tissue/ml and used immediately in the binding assay. Aliquots of final membrane preparations were also stored at −80 C until required for protein determination. All centrifugations will be carried out at 4° C.

To examine binding, the frontal cortical membranes (400 μl; equivalent to 2.5 mg wet weight of tissue/tube) were incubated with 50 μl of [3H] MDL 100907 (8 concentrations: 0.025, 0.05, 0.1, 0.2, 0.4, 0.8, 1.6, 3.2 nM) and either 50 μl of buffer (total binding) or 50 μl of 10 μM ketanserin (to define nonspecific binding) for 60 min at 25 C. The homogenate, assay and wash buffer consisted of 50 mM Tris, pH 7.4. For each animal, for each radioligand concentration, there will be two tubes for the determination of total binding and one tube for the determination of non-specific binding. This assay was undertaken once.

Membrane bound radioactivity was recovered by filtration under vacuum through Skatron 11731 filters, presoaked in 0.5% polyethylenimine (PEI) using a Skatron cell harvester. Filters were rapidly washed with ice cold wash buffer (wash setting 9,9,0) and radioactivity determined by liquid scintillation counting (1 ml Packard MV Gold scintillator). A protein assay (Thermo Scientific Pierce BCA Protein Assay Kit, 23225) was performed on the final membrane preparation for the determination of the number of receptors in fmoles/mg protein.

A trend increase in psilocybin HTR was observed 2 h but not 24 h post diazepam pretreatment. A trend increase in 5HT2A binding was observed with diazepam pretreatment when examined 24 h post final administration. Diazepam pretreatment produces a trend increase in the HTR over 30 minutes, and significant increase in HTR when HTR at 10-15 and 20-25 minutes are analyzed separately. This could reflect a potentiation of the psychedelic effects of psilocybin.

TABLE 20

| | | Psilocybin challenge | |
|---|---|---|---|
| Group | Treatment | (0.3 mg/kg, s.c.) | n |
| A | Diazepam i.p 1.25 mg/kg | 2 h post diazepam | 12 |
| | twice daily for 14 days | | |
| B | Saline i.p twice daily | 2 h post saline | 12 |
| | for 14 days | | |
| C | Diazepam i.p 1.25 mg/kg | 24 h post diazepam | 12 |
| | twice daily for 14 days | | |
| D | Saline i.p twice daily | 24 h post saline | 12 |
| | for 14 days | | |
| E | Diazepam i.p 1.25 mg/kg | Brain tissue collection for | 6 |
| | twice daily for 14 days | 5HT2A Kd and Bmax assess- | |
| | | ment 24 h post last dose of | |
| | | diazepam (no psilocybin) | |
| F | Saline i.p twice daily | Brain tissue collection for | 6 |
| | for 14 days | 5HT2A Kd and Bmax assess- | |
| | | ment 24 h post last dose of | |
| | | diazepam (no psilocybin) | |

### Experimental Data

TABLE 21
Mean head twitch responses during the 30 minutes post psilocybin challenge.

| | Means | | | | | | | | |
|---|---|---|---|---|---|---|---|---|---|
| | 0-5 | 5-10 | 10-15 | 15-20 | 20-25 | 25-30 | 0-15 | 15-30 | 0-30 |
| | mins | mins | mins | mins | mins | mins | mins | mins | mins |
| Diazepam | 1.07 | 0.58 | 2.09 | 0.73 | 1.53 | 0.49 | 4.04 | 3.50 | 7.80 |
| 1.25 mg/ | | | | | | | | | |
| kg ip bid/ | | | | | | | | | |
| Psilocybin | | | | | | | | | |
| 0.3 mg/kg | | | | | | | | | |
| sc, 2 hr | | | | | | | | | |
| post Diazepam | | | | | | | | | |
| (A) | | | | | | | | | |
| Saline ip bid/ | 0.76 | 0.81 | 0.81 | 0.60 | 0.32 | 0.80 | 2.93 | 2.35 | 5.78 |
| Psilocybin | | | | | | | | | |
| 0.3 mg/kg | | | | | | | | | |
| sc, 2 hr | | | | | | | | | |
| post saline | | | | | | | | | |
| (B) | | | | | | | | | |
| Diazepam | 1.41 | 1.48 | 0.65 | 1.64 | 0.85 | 0.50 | 4.30 | 3.89 | 8.44 |
| 1.25 mg/ | | | | | | | | | |
| kg ip bid/ | | | | | | | | | |
| Psilocybin | | | | | | | | | |
| 0.3 mg/kg | | | | | | | | | |
| sc, 24 hr | | | | | | | | | |
| post Diazepam | | | | | | | | | |
| (C) | | | | | | | | | |
| Saline ip bid/ | 1.81 | 2.25 | 1.04 | 1.70 | 0.43 | 1.16 | 5.65 | 3.60 | 9.70 |
| Psilocybin | | | | | | | | | |
| 0.3 mg/kg | | | | | | | | | |
| sc, 24 hr | | | | | | | | | |
| post saline | | | | | | | | | |
| (D) | | | | | | | | | |
| SEMS | | | | | | | | | |
| A | 0.32 | 0.28 | 0.42 | 0.40 | 0.26 | 0.24 | 0.60 | 0.64 | 1.05 |
| B | 0.33 | 0.36 | 0.26 | 0.29 | 0.18 | 0.35 | 0.78 | 0.57 | 1.15 |
| C | 0.31 | 0.24 | 0.27 | 0.45 | 0.18 | 0.27 | 0.55 | 0.51 | 0.73 |
| D | 0.33 | 0.43 | 0.31 | 0.33 | 0.19 | 0.44 | 0.75 | 0.70 | 1.13 |
| p-vals | | | | | | | | | |
| A | 0.468 | 0.559 | 0.020 * | 0.772 | <0.001*** | 0.495 | 0.239 | 0.180 | 0.165 |
| C | 0.482 | 0.231 | 0.342 | 0.932 | 0.172 | 0.195 | 0.231 | 0.761 | 0.452 |

TABLE 22
[3H]MDL100907 binding in animals pretreated with saline (+24 h)

| GROUP F: Saline | | | | | |
|---|---|---|---|---|---|
| i.p. BID | | | | Bmax | |
| for 14 days | Kd | Bmax | Protein | (fmoles/mg | |
| Animal ID | (nM) | (pM) | (mg/ml) | protein) | R² |
| 7F | 0.25 | 64.4 | 0.26 | 252 | 0.98 |
| 8F | 0.20 | 58.9 | 0.23 | 251 | 0.96 |
| 9F | 0.21 | 89.8 | 0.26 | 342 | 0.91 |
| 31F | 0.20 | 82.9 | 0.32 | 260 | 0.95 |
| 32F | 0.18 | 91.9 | 0.27 | 341 | 0.96 |
| 33F | 0.18 | 87.2 | 0.28 | 312 | 0.93 |
| Mean | 0.20 | 79.2 | 0.270 | 293 | |
| SEM | 0.011 | 5.72 | 0.01 | 17.9 | |

TABLE 23
[3H]MDL100907 binding in animals pretreated
with diazepam (+24 h)

| GROUP E: | | | | | |
|---|---|---|---|---|---|
| Diazepam 1.25 | | | | | |
| mg/kg i.p. BID | | | | Bmax | |
| for 14 days | Kd | Bmax | Protein | (fmoles/mg | |
| Animal ID | (nM) | (pM) | (mg/ml) | protein) | R² |
| 16E | 0.18 | 74.0 | 0.27 | 271 | 0.93 |
| 17E | 0.19 | 78.3 | 0.23 | 339 | 0.94 |
| 18E | 0.18 | 77.8 | 0.23 | 334 | 0.94 |
| 40E | 0.20 | 93.6 | 0.26 | 360 | 0.97 |
| 41E | 0.21 | 87.2 | 0.28 | 314 | 0.97 |
| 42E | 0.20 | 87.6 | 0.23 | 376 | 0.92 |
| Mean | 0.19 | 83.1 | 0.25 | 332 | |
| SEM | 0.005 | 3.06 | 0.009 | 15.1 | |
| p (unpaired | 0.411 | 0.561 | 0.217 | 0.121 | |
| t-test) | | | | | |

Additional Examples are provided in PCT/IB2020/053688 (published as WO2020/212952), which is incorporated by reference herein in its entirety.

### EMBODIMENTS

1. A method of administering psilocybin to a subject in need thereof, wherein prior to administration of psilocybin the subject was on an antidepressive (AD) therapy regimen comprising
a) ceasing AD therapy 1 to 35 days prior to administration of psilocybin;
b) administering one or more benzodiazepines at least once daily to the subject starting at least 1 to 35 days prior to administration of psilocybin; and
c) administering psilocybin to the subject.

2. The method of embodiment 1, wherein AD therapy is ceased during a titration period, wherein during the titration period the dose of AD is reduced from a maintenance dose to cessation.

3. The method of embodiment 2, wherein one or more benzodiazepines is administered during the AD titration period.

4. The method of embodiment 2, wherein one or more benzodiazepines is administered after cessation of the AD.

5. The method of embodiment 2, wherein one or more benzodiazepines is administered before the AD titration period.

6. The method of embodiment 1, wherein AD therapy is ceased immediately, wherein immediate cessation of an AD does not comprise a titration period.

7. The method of embodiment 6, wherein one or more benzodiazepines are administered after cessation of AD therapy.

8. The method of embodiment 6, wherein one or more benzodiazepines are administered before cessation of AD therapy.

9. The method of any one of embodiments 1, 2, 4, 6, or 7, wherein AD therapy is ceased 29 to 35 days prior to administration of psilocybin and wherein one or more benzodiazepines are administered starting 1 to 28 days prior to administration of psilocybin.

10. The method of any one of embodiments 1, 2, 4, 6, or 7, wherein AD therapy is ceased 22 to 35 days prior to administration of psilocybin and wherein one or more benzodiazepines are administered starting 1 to 21 days prior to administration of psilocybin.

11. The method of any one of embodiments 1, 2, 4, 6, or 7, wherein AD therapy is ceased 15 to 35 days prior to administration of psilocybin and wherein one or more benzodiazepines are administered starting 1 to 14 days prior to administration of psilocybin.

12. The method of any one of embodiments 1, 2, 4, 6, or 7, wherein AD therapy is ceased 8 to 35 days prior to administration of psilocybin and wherein one or more benzodiazepines are administered starting 1 to 7 days prior to administration of psilocybin.

13. The method of any one of embodiments 1, 2, 5, 6, or 8, wherein AD therapy is ceased 29 to 35 days prior to administration of psilocybin and wherein one or more benzodiazepines are administered starting at least 35 days before administration of psilocybin.

14. The method of any one of embodiments 1, 2, 5, 6, or 8, wherein AD therapy is ceased 22 to 35 days prior to administration of psilocybin and wherein one or more benzodiazepines are administered starting at least 35 days before administration of psilocybin.

15. The method of any one of embodiments 1, 2, 5, 6, or 8, wherein AD therapy is ceased 15 to 35 days prior to administration of psilocybin and wherein one or more benzodiazepines are administered starting at least 35 days before administration of psilocybin.

16. The method of any one of embodiments 1, 3, 6, 7, or 9, wherein AD therapy is ceased 8 to 35 days prior to administration of psilocybin and wherein one or more benzodiazepines are administered starting at least 35 days before administration of psilocybin.

17. The method of any one of the preceding embodiments, wherein AD therapy is ceased in 1 to 35 days prior to administration of psilocybin and wherein one or more benzodiazepines is administered 1 to 28 days prior to administration of psilocybin.

18. The method of any one of the preceding embodiments, wherein AD therapy is ceased in 1 to 35 days prior to administration of psilocybin and wherein one or more benzodiazepines is administered 1 to 14 days prior to administration of psilocybin.

19. The method of any one of the preceding embodiments, wherein AD therapy is ceased in 1 to 35 days prior to administration of psilocybin and wherein one or more benzodiazepines is administered 1 to 7 days prior to administration of psilocybin.

20. The method of any one of the preceding embodiments, wherein AD therapy is ceased in 1 to 28 days prior to administration of psilocybin and wherein one or more benzodiazepines is administered 1 to 35 days prior to administration of psilocybin.

21. The method of any one of the preceding embodiments, wherein AD therapy is ceased in 1 to 28 days prior to administration of psilocybin and wherein one or more benzodiazepines is administered 1 to 28 days prior to administration of psilocybin.

22. The method of any one of the preceding embodiments, wherein AD therapy is ceased in 1 to 28 days prior to administration of psilocybin and wherein one or more benzodiazepines is administered 1 to 14 days prior to administration of psilocybin.

23. The method of any one of the preceding embodiments, wherein AD therapy is ceased in 1 to 28 days prior to administration of psilocybin and wherein one or more benzodiazepines is administered 1 to 7 days prior to administration of psilocybin.

24. The method of any one of the preceding embodiments, wherein AD therapy is ceased in 1 to 14 days prior to administration of psilocybin and wherein one or more benzodiazepines is administered 1 to 35 days prior to administration of psilocybin.

25. The method of any one of the preceding embodiments, wherein AD therapy is ceased in 1 to 14 days prior to administration of psilocybin and wherein one or more benzodiazepines is administered 1 to 28 days prior to administration of psilocybin.

26. The method of any one of the preceding embodiments, wherein AD therapy is ceased in 1 to 14 days prior to administration of psilocybin and wherein one or more benzodiazepines is administered 1 to 21 days prior to administration of psilocybin.

27. The method of any one of the preceding embodiments, wherein AD therapy is ceased in 1 to 14 days prior to administration of psilocybin and wherein one or more benzodiazepines is administered 1 to 14 days prior to administration of psilocybin.

28. The method of any one of the preceding embodiments, wherein AD therapy is ceased in 1 to 14 days prior to administration of psilocybin and wherein one or more benzodiazepines is administered 1 to 7 days prior to administration of psilocybin.

29. The method of any one of the preceding embodiments, wherein AD therapy is ceased in 1 to 7 days prior to administration of psilocybin and wherein one or more benzodiazepines is administered 1 to 35 days prior to administration of psilocybin.

30. The method of any one of the preceding embodiments, wherein AD therapy is ceased in 1 to 7 days prior to administration of psilocybin and wherein one or more benzodiazepines is administered 1 to 28 days prior to administration of psilocybin.

31. The method of any one of the preceding embodiments, wherein AD therapy is ceased in 1 to 7 days prior to administration of psilocybin and wherein one or more benzodiazepines is administered 1 to 21 days prior to administration of psilocybin.

32. The method of any one of the preceding embodiments, wherein AD therapy is ceased in 1 to 7 days prior to administration of psilocybin and wherein one or more benzodiazepines is administered 1 to 14 days prior to administration of psilocybin.

33. The method of any one of the preceding embodiments, wherein AD therapy is ceased in 1 to 7 days prior to administration of psilocybin and wherein one or more benzodiazepines is administered 1 to 7 days prior to administration of psilocybin.

34. The method of embodiment 2, comprising reducing the maintenance AD daily dose by at least about 10% every three to four days.

35. The method of embodiment 2, comprising reducing the maintenance AD daily dose by at least about 25% every three to four days.

36. The method of embodiment 2, comprising reducing the maintenance AD daily dose by at least about 50% every three to four days.

37. The method of embodiment 2, comprising reducing the maintenance AD daily dose by at least about 10% every week.

38. The method of embodiment 2, comprising reducing the maintenance AD daily dose by at least about 25% every week.

39. The method of embodiment 2, comprising reducing the maintenance AD daily dose by at least about 50% every week.

40. The method of embodiment 2, comprising reducing the maintenance AD daily dose by at least about 10% every other week.

41. The method of embodiment 2, comprising reducing the maintenance AD daily dose by at least about 25% every other week.

42. The method of embodiment 2, comprising reducing the maintenance AD daily dose by at least about 50% every other week.

43. The method of any one of embodiments 34-42, wherein reducing the maintenance AD daily dose starts between 1 and 16 weeks before administration of psilocybin.

44. The method of any one of embodiments 34-42, wherein reducing the maintenance AD daily dose starts between 1 and 12 weeks before administration of psilocybin.

45. The method of any one of embodiments 34-42, wherein reducing the maintenance AD daily dose starts between 1 and 8 weeks before administration of psilocybin.

46. The method of any one of embodiments 34-42, wherein reducing the maintenance AD daily dose starts between 1 and 4 weeks before administration of psilocybin.

47. The method of any one of embodiments 34-42, wherein reducing the maintenance AD daily dose starts between 1 and 2 weeks before administration of psilocybin.

48. The method of embodiment 1, wherein the AD is ceased 14 days prior to administration of psilocybin.

49. The method of embodiment 2, wherein the AD titration period comprises:
a) administering to the subject 60-90% of the subject's maintenance dose of one or more ADs for 21 to 35 days prior to administration of psilocybin;
b) administering to the subject 40-60% of the subject's maintenance dose of one or more ADs for 14 to 20 days prior to administration of psilocybin;
c) administering to the subject 25-40% of the subject's maintenance dose of one or more ADs for 7 to 13 days prior to administration of psilocybin; and
d) administering to the subject 5-25% of the subject's maintenance dose of one or more ADs for 1 to 6 days prior to administration of psilocybin.

50. The method of embodiment 2, wherein the AD titration period comprises:
a) administering to the subject 60-90% of the subject's maintenance dose of one or more ADs for 21 to 35 days prior to administration of psilocybin;
b) administering to the subject 30-60% of the subject's maintenance dose of one or more ADs for 14 to 20 days prior to administration of psilocybin;
c) administering to the subject 5-30% of the subject's maintenance dose of one or more ADs for 7 to 13 days prior to administration of psilocybin; and
d) ceasing AD therapy 6 days prior to administration of psilocybin.

51. The method of any of the preceding embodiments, wherein the AD is an SSRI selected from the group consisting of citalopram, escitalopram, paroxetine, sertraline, fluvoxamine, fluoxetine, and combinations thereof.

52. The method of any of the preceding embodiments, wherein the benzodiazepine is selected from the group consisting of alprazolam, chlordiazepoxide, clonazepam, diazepam, lorazepam, oxazepam, and combinations thereof.

53. The method of any of the preceding embodiments, wherein at least one side effect of AD washout is reduced.

54. The method of embodiment 52, wherein the at least one side effect of AD washout is selected from the group consisting of headache, asthenia, flu syndrome, fever, vasodilation, nausea, diarrhea, anorexia, dry mouth, dyspepsia, constipation, flatulence, vomiting, weight loss, insomnia, nervousness, anxiety, somnolence, dizziness, tremor, decreased libido, abnormal thinking, sweating, rash, pruritus, abnormal vision, dyspepsia, abdominal pain, fatigue, arthralgia, myalgia, somnolence, agitation, dysmenorrhea, decreased libido, yawning, upper respiratory tract infection, rhinitis, sinusitis, ejaculation disorder, ejaculatory delay, impotence, dysphoric mood, irritability, agitation, dizziness, sensory disturbances (e.g. paresthesias such as electric shock sensations), confusion, lethargy, emotional lability, and hypomania.

55. The method of any of the preceding embodiments, wherein the AD is an SSRI.

56. The method of any of the preceding embodiments, wherein the AD is a tricyclic AD.

57. The method of embodiment 56, wherein the tricyclic AD is selected from the group consisting of amitriptyline, imipramine, and nortriptyline.

58. The method of any of the preceding embodiments, wherein the AD is a serotonin norepinephrine reuptake inhibitor (SNRI).

59. The method of embodiment 58, wherein the SNRI is selected from the group consisting of venlafaxine and duloxetine.

60. The method of any of the preceding embodiments, wherein the AD is a monoamine oxidase inhibitor.

61. The method of embodiment 60, wherein the monoamine oxidase inhibitor is selected from the group consisting of phenelzine and tranylcypromine.

62. The method of any of the preceding embodiments, wherein the AD is an atypical antipsychotic.

63. The method of embodiment 62, wherein the atypical antipsychotic is selected from the group consisting of mianserin, lurasidone, aripiprazole, risperidone, olanzapine, quetiapine, ziprasidone, clozapine, iloperidone, paliperidone, asenapine, and olanzapine/fluoxetine.

64. The method of any of the preceding embodiments, wherein the AD is selected from the following group consisting of amitriptyline, amoxapine, clomipramine, desipramine, dosulepin, doxepin, imipramine, lofepramine, nortriptyline, protriptyline, tianeptine, trimipramine, isocarboxazid, phenelzine, tranylcypromine, moclobemide, selegiline, maprotiline, mianserin, mirtazapine, nefazadone, trazodone, vilazodone, vortioxetine, bupropion, agomelatine, flupentixol, ketamine, and mixtures thereof.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

The foregoing is illustrative of the present invention and is not to be construed as limiting thereof. The invention is defined by the following claims, with equivalents of the claims to be included therein.

What is claimed is:

1. A method of administering psilocybin to a subject in need thereof, wherein prior to administration of psilocybin the subject was on a selective serotonin reuptake inhibitor (SSRI) therapy regimen comprising
a) ceasing SSRI therapy 1 to 35 days prior to administration of psilocybin;
b) administering one or more benzodiazepines at least once daily to the subject starting at least 1 to 35 days prior to administration of psilocybin; and
c) administering psilocybin to the subject.

2. The method of claim 1, wherein SSRI therapy is ceased during a titration period, wherein during the titration period the dose of SSRI is reduced from a maintenance dose to cessation.

3. The method of claim 2, wherein one or more benzodiazepines is administered during the SSRI titration period.

4. The method of claim 2, wherein one or more benzodiazepines is administered after cessation of the SSRI.

5. The method of claim 2, wherein one or more benzodiazepines is administered before the SSRI titration period.

6. The method of claim 1, wherein SSRI therapy is ceased immediately, wherein immediate cessation of an SSRI does not comprise a titration period.

7. The method of claim 6, wherein one or more benzodiazepines are administered after cessation of SSRI therapy.

8. The method of claim 6, wherein one or more benzodiazepines are administered before cessation of SSRI therapy.

9. The method of any one of claim 1, 2, 4, 6, or 7, wherein SSRI therapy is ceased 29 to 35 days prior to administration of psilocybin and wherein one or more benzodiazepines are administered starting 1 to 28 days prior to administration of psilocybin.

10. The method of any one of claim 1, 2, 4, 6, or 7, wherein SSRI therapy is ceased 22 to 35 days prior to administration of psilocybin and wherein one or more benzodiazepines are administered starting 1 to 21 days prior to administration of psilocybin.

11. The method of any one of claim 1, 2, 4, 6, or 7, wherein SSRI therapy is ceased 15 to 35 days prior to administration of psilocybin and wherein one or more benzodiazepines are administered starting 1 to 14 days prior to administration of psilocybin.

12. The method of any one of claim 1, 2, 4, 6, or 7, wherein SSRI therapy is ceased 8 to 35 days prior to administration of psilocybin and wherein one or more benzodiazepines are administered starting 1 to 7 days prior to administration of psilocybin.

13. The method of any one of claim 1, 2, 5, 6, or 8, wherein SSRI therapy is ceased 29 to 35 days prior to administration of psilocybin and wherein one or more benzodiazepines are administered starting at least 35 days before administration of psilocybin.

14. The method of any one of claim 1, 2, 5, 6, or 8, wherein SSRI therapy is ceased 22 to 35 days prior to administration of psilocybin and wherein one or more benzodiazepines are administered starting at least 35 days before administration of psilocybin.

15. The method of any one of claim 1, 2, 5, 6, or 8, wherein SSRI therapy is ceased 15 to 35 days prior to administration of psilocybin and wherein one or more benzodiazepines are administered starting at least 35 days before administration of psilocybin.

16. The method of any one of claim 1, 3, 6, 7, or 9, wherein SSRI therapy is ceased 8 to 35 days prior to administration of psilocybin and wherein one or more benzodiazepines are administered starting at least 35 days before administration of psilocybin.

17. The method of any one of the preceding claims, wherein SSRI therapy is ceased in 1 to 35 days prior to administration of psilocybin and wherein one or more benzodiazepines is administered 1 to 28 days prior to administration of psilocybin.

18. The method of any one of the preceding claims, wherein SSRI therapy is ceased in 1 to 35 days prior to administration of psilocybin and wherein one or more benzodiazepines is administered 1 to 14 days prior to administration of psilocybin.

19. The method of any one of the preceding claims, wherein SSRI therapy is ceased in 1 to 35 days prior to administration of psilocybin and wherein one or more benzodiazepines is administered 1 to 7 days prior to administration of psilocybin.

20. The method of any one of the preceding claims, wherein SSRI therapy is ceased in 1 to 28 days prior to administration of psilocybin and wherein one or more benzodiazepines is administered 1 to 35 days prior to administration of psilocybin.

21. The method of any one of the preceding claims, wherein SSRI therapy is ceased in 1 to 28 days prior to administration of psilocybin and wherein one or more benzodiazepines is administered 1 to 28 days prior to administration of psilocybin.

22. The method of any one of the preceding claims, wherein SSRI therapy is ceased in 1 to 28 days prior to administration of psilocybin and wherein one or more benzodiazepines is administered 1 to 14 days prior to administration of psilocybin.

23. The method of any one of the preceding claims, wherein SSRI therapy is ceased in 1 to 28 days prior to administration of psilocybin and wherein one or more benzodiazepines is administered 1 to 7 days prior to administration of psilocybin.

24. The method of any one of the preceding claims, wherein SSRI therapy is ceased in 1 to 14 days prior to administration of psilocybin and wherein one or more benzodiazepines is administered 1 to 35 days prior to administration of psilocybin.

25. The method of any one of the preceding claims, wherein SSRI therapy is ceased in 1 to 14 days prior to administration of psilocybin and wherein one or more benzodiazepines is administered 1 to 28 days prior to administration of psilocybin.

26. The method of any one of the preceding claims, wherein SSRI therapy is ceased in 1 to 14 days prior to administration of psilocybin and wherein one or more benzodiazepines is administered 1 to 21 days prior to administration of psilocybin.

27. The method of any one of the preceding claims, wherein SSRI therapy is ceased in 1 to 14 days prior to administration of psilocybin and wherein one or more benzodiazepines is administered 1 to 14 days prior to administration of psilocybin.

28. The method of any one of the preceding claims, wherein SSRI therapy is ceased in 1 to 14 days prior to administration of psilocybin and wherein one or more benzodiazepines is administered 1 to 7 days prior to administration of psilocybin.

29. The method of any one of the preceding claims, wherein SSRI therapy is ceased in 1 to 7 days prior to administration of psilocybin and wherein one or more benzodiazepines is administered 1 to 35 days prior to administration of psilocybin.

30. The method of any one of the preceding claims, wherein SSRI therapy is ceased in 1 to 7 days prior to administration of psilocybin and wherein one or more benzodiazepines is administered 1 to 28 days prior to administration of psilocybin.

31. The method of any one of the preceding claims, wherein SSRI therapy is ceased in 1 to 7 days prior to administration of psilocybin and wherein one or more benzodiazepines is administered 1 to 21 days prior to administration of psilocybin.

32. The method of any one of the preceding claims, wherein SSRI therapy is ceased in 1 to 7 days prior to administration of psilocybin and wherein one or more benzodiazepines is administered 1 to 14 days prior to administration of psilocybin.

33. The method of any one of the preceding claims, wherein SSRI therapy is ceased in 1 to 7 days prior to administration of psilocybin and wherein one or more benzodiazepines is administered 1 to 7 days prior to administration of psilocybin.

34. The method of claim 2, comprising reducing the maintenance SSRI daily dose by at least about 10% every three to four days.

35. The method of claim 2, comprising reducing the maintenance SSRI daily dose by at least about 25% every three to four days.

36. The method of claim 2, comprising reducing the maintenance SSRI daily dose by at least about 50% every three to four days.

37. The method of claim 2, comprising reducing the maintenance SSRI daily dose by at least about 10% every week.

38. The method of claim 2, comprising reducing the maintenance SSRI daily dose by at least about 25% every week.

39. The method of claim 2, comprising reducing the maintenance SSRI daily dose by at least about 50% every week.

40. The method of claim 2, comprising reducing the maintenance SSRI daily dose by at least about 10% every other week.

41. The method of claim 2, comprising reducing the maintenance SSRI daily dose by at least about 25% every other week.

42. The method of claim 2, comprising reducing the maintenance SSRI daily dose by at least about 50% every other week.

43. The method of any one of claims 34-42, wherein reducing the maintenance SSRI daily dose starts between 1 and 16 weeks before administration of psilocybin.

44. The method of any one of claims 34-42, wherein reducing the maintenance SSRI daily dose starts between 1 and 12 weeks before administration of psilocybin.

45. The method of any one of claims 34-42, wherein reducing the maintenance SSRI daily dose starts between 1 and 8 weeks before administration of psilocybin.

46. The method of any one of claims 34-42, wherein reducing the maintenance SSRI daily dose starts between 1 and 4 weeks before administration of psilocybin.

47. The method of any one of claims 34-42, wherein reducing the maintenance SSRI daily dose starts between 1 and 2 weeks before administration of psilocybin.

48. The method of claim 1, wherein the SSRI is ceased 14 days prior to administration of psilocybin.

49. The method of claim 2, wherein the SSRI titration period comprises:
a) administering to the subject 60-90% of the subject's maintenance dose of one or more SSRIs for 21 to 35 days prior to administration of psilocybin;
b) administering to the subject 40-60% of the subject's maintenance dose of one or more SSRIs for 14 to 20 days prior to administration of psilocybin;
c) administering to the subject 25-40% of the subject's maintenance dose of one or more SSRIs for 7 to 13 days prior to administration of psilocybin; and
d) administering to the subject 5-25% of the subject's maintenance dose of one or more SSRIs for 1 to 6 days prior to administration of psilocybin.

50. The method of claim 2, wherein the SSRI titration period comprises:
a) administering to the subject 60-90% of the subject's maintenance dose of one or more SSRIs for 21 to 35 days prior to administration of psilocybin;
b) administering to the subject 30-60% of the subject's maintenance dose of one or more SSRIs for 14 to 20 days prior to administration of psilocybin;
c) administering to the subject 5-30% of the subject's maintenance dose of one or more SSRIs for 7 to 13 days prior to administration of psilocybin; and
d) ceasing SSRI therapy 6 days prior to administration of psilocybin.

51. The method of any of the preceding claims, wherein the SSRI is selected from the group consisting of citalopram, escitalopram, paroxetine, sertraline, fluvoxamine, fluoxetine, and combinations thereof.

52. The method of any of the preceding claims, wherein the benzodiazepine is selected from the group consisting of alprazolam, chlordiazepoxide, clonazepam, diazepam, lorazepam, oxazepam, and combinations thereof.

53. The method of any of the preceding claims, wherein at least one side effect of SSRI washout is reduced.

54. The method of claim 52, wherein the at least one side effect of SSRI washout is selected from the group consisting of headache, asthenia, flu syndrome, fever, vasodilation, nausea, diarrhea, anorexia, dry mouth, dyspepsia, constipation, flatulence, vomiting, weight loss, insomnia, nervousness, anxiety, somnolence, dizziness, tremor, decreased libido, abnormal thinking, sweating, rash, pruritus, abnormal vision, dyspepsia, abdominal pain, fatigue, arthralgia, myalgia, somnolence, agitation, dysmenorrhea, decreased libido, yawning, upper respiratory tract infection, rhinitis, sinusitis, ejaculation disorder, ejaculatory delay, impotence, dysphoric mood, irritability, agitation, dizziness, sensory disturbances (e.g. paresthesias such as electric shock sensations), confusion, lethargy, emotional lability, and hypomania.
